(19) United States
(12) Patent Application Publication
(10) Pub. No.: US 2003/0007968 A1
(43) Pub. Date: Jan. 9, 2003

(54) Methods of inducing organ transplant tolerance and correcting hemoglobinopathies

(76) Inventors: Christian P. Larsen, Atlanta, GA (US); Thomas C. Pearson, Atlanta, GA (US); Edmund K. Waller, Atlanta, GA (US); Andrew B. Adams, Atlanta, GA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/057,288

(22) Filed: Jan. 25, 2002

Related U.S. Application Data
(60) Provisional application No. 60/264,528, filed on Jan. 26, 2001.
(60) Provisional application No. 60/303,142, filed on Jul. 5, 2001.

(51) Int. Cl. A61K 39/395; A61K 31/255; A61K 45/00

(52) U.S. Cl. 424/144.1; 424/93.7; 514/517

(57) ABSTRACT

Methods of establishing hematopoietic chimerism useful to correct hematological diseases and promote acceptance of organ transplants include administering busulfan, costimulation blockade, and readily attainable numbers of T-cell depleted bone marrow cells.

This application is based on provisional applications, U.S. Serial No. 60/264,528, filed Jan. 26, 2001, and No. 60/303,142, filed Jul. 5, 2001, the contents of which are hereby incorporated by reference, in their entirety, into this application.

The invention disclosed herein was made with government support under Grant Nos. DK/AI40519, CA74364-03, and AI44644, awarded by the National Institutes of Health. The government may have certain rights in this invention.

Throughout this application various publications are referenced. The disclosures of these publications in their entireties are hereby incorporated by reference into this application in order to more filly describe the state of the art to which the invention pertains.

### FIELD OF THE INVENTION

The present invention relates to methods of establishing mixed hematopoietic chimerism in subjects. More specifically the present invention encompasses methods for inhibiting rejection of organ or tissue/cell transplants, methods for inducing immunological tolerance in subjects receiving an organ or tissue transplant, and methods for treating subjects with hemoglobinopathies.

### BACKGROUND OF THE INVENTION

Transplantation has emerged as a preferred method of treatment for many forms of end-stage organ failure. Improved results in clinical transplantation have been achieved primarily through the development of increasingly potent non-specific immunosuppressive drugs to inhibit rejection responses (Lancet, 345:1321-1325 (1995)).

While short-term results have improved, long-term outcomes remain inadequate. Currently, life-long immunosuppressive agents are required to combat chronic rejection of the transplanted organ and the use of these agents dramatically increases the risks of cardiovascular disease, infections and malignancies. The development of strategies to promote the acceptance of allogeneic tissues without the need for chronic immunosuppression should not only reduce the risk of these life-threatening complications, but also greatly expand the application of organ, tissue and cellular transplantation for diseases such as the hemoglobinopathies, genetic immunodeficiencies, and possibly autoimmune diseases.

Mixed hematopoietic chimerism induces a state of immunological tolerance (Owen, Science, 102:400-401 (1945); Billingham et al., Nature, 172:603-606 (1953)). Many protocols for inducing hematopoietic chimerism require conditioning regimens, including gamma irradiation and/or depletion of the peripheral immune system. (Ildstad et al., Nature, 307:168-170 (1984); Sharabi et al., J. Exp. Med., 169:493-502 (1989); Tomita et al., J. Immunol., 153:1087-1098 (1994); Mayumi et al., J. Exp. Med., 169:213-238 (1989); and U.S. Pat. Nos. 5,876,692 and 6,217,867). Unfortunately, concerns over toxicity associated with these regimens, such as the potential for over-immunosuppression and/or loss of memory that may occur with peripheral T cell depletion, or the enhanced risk of malignancy with whole body irradiation may limit the clinical application of these approaches for the correction of hematologic diseases or for the induction of solid organ transplant tolerance.

Simultaneous blockade of costimulatory signals and administration of supra-physiological doses of non-T cell depleted donor bone marrow obviate the need for pre-transplant conditioning (Durham et al., Journal of Immunology, 165:1-4 (2000); Wekerle et al., Nature Medicine, 6:464-469 (2000)). However, these protocols require quantities of non-T cell depleted bone marrow that are presently clinically unfeasible to attain and the degree of donor chimerism achieved may be too low to effectively treat hemoglobinopathies. In addition, these protocols rely upon the use of unseparated bone marrow cells. Specifically, T cells are not removed from the preparations. While leaving T cells in the preparation may enhance hematopoietic stem cell engraftment, the risk of potentially lethal graft versus host disease is proportional to the T cell mass in the bone marrow innoculum. Though the percentage of T cells in the bone marrow is relatively low, the mega doses of bone marrow required for these protocols transfers vastly more T cells than the current methods employed in clinical bone marrow transplantation. Furthermore, the degree of donor chimerism achieved by these protocols may be too low to effectively treat, or correct the pathophysiology of, hemoglobinopathies, such as sickle cell anemia and the thalassemias.

Thalassemia is a genetic disorder involving abnormal patterns of hemoglobin chain synthesis. The first successful report of a bone marrow transplantation to correct thalassemia was demonstrated in 1982 (Thomas et al., Lancet, 2:227-229 (1982)). Busulfan is commonly used in a multi-dose fashion in conjunction with other chemotherapeutic agents for recipient conditioning in many clinical bone marrow transplant regimens (Brodsky et al., Cancer Invest., 7:509-513 (1989)). Busulfan is an alkylating agent that produces a specific loss of early hematopoietic stem cells, and is often used as an anti-proliferative, chemotherapeutic agent, (Santos et al., Human bone marrow transplantation. Washington, American Assoc. of Blood Banks, (1976); Basch et al., Stem Cells, 15:314-323 (1997)). Busulfan can be used with the alkylating agent, cyclophosphamide, to facilitate engraftment of bone marrow cells and establish chimerism in thalassemic patients (Lucarelli et al., Ann NY Acad Sci, 445:428-431 (1985); Mentzer and Cowan, J. Pediatr. Hematol Oncol, 22(6):598-601 (2000)). In addition, busulfan has been used in subablative doses to promote engraftment of stem cells in syngeneic murine models (Yeager et al., Bone Marrow Transplant., 9:199-204 (1992)). Similarly, as disclosed in U.S. Pat. No. 6,217,867, cyclophosphamide and total body irradiation can be used to achieve engraftment of bone marrow, but engraftment was not achieved with cyclosphosphamide alone. Although the protocols in the studies above were somewhat successful in correcting thalassemia, these protocols are toxic to patients. Thus, it is desirable to develop protocols that are less toxic to patients.

Sickle cell disease (SCD) is a genetic disorder involving a mutation in the amino acid sequence of hemoglobin. People with sickle cell disease suffer from both episodic acute complications and chronic, progressive, multi-system decline. Although medical treatments are life-extending, only stem cell transplantation offers an effective cure. There are, however, currently two major barriers to stem cell transplantation for sickle cell disease: (1) the high morbidity and mortality associated with conventional bone marrow transplantation, as discussed above, and (2) the scarcity of acceptable stem cell donors (Walters et al., Biol. Blood Marrow Transplant., 2:100-104 (1996); Platt et al., New England. J. Med., 335:426-428 (1996)).

Conventional bone marrow transplantation can cure sickle cell disease, but requires toxic myeloablative preconditioning regimens in order to achieve donor cell engraftment (Walters et al., Blood, 95:1918-1924 (2000); Vermylen et al. Bone Marrow Transplant., 22:1-6 (1998)). These intensive preparative regimens have many toxic side effects, including potential organ failure and a long-term risk of malignancy. In certain patient populations, the morbidity and mortality of transplant can outweigh the morbidity and mortality of sickle cell disease (Platt et al., New England. J. Med., 335:426-428 (1996)). A dilemma is now developing between early treatment with stem cell transplantation (shown to increase survival and disease-free survival when compared to transplantation after more disease-related complications have occurred) and a delayed approach, during which medical management ameliorates the symptoms of sickle cell disease until a later age when definitive therapy can be instituted (Walters et al., Biol. Blood Marrow Transplant., 2:100-104 (1996); Platt et al., New England. J. Med., 335:426-428 (1996)). Unfortunately, this latter course may decrease the chance of successful stem cell transplant.

The paucity of matched-related donors has severely limited the number of sickle cell disease patients eligible for transplantation. In fact, in the Seattle consortium study, only 6.5% of potential sickle cell disease patients were found to be eligible for stem cell transplantation based on disease severity, and of these only 14% had an HLA-matched-related donor (Walters et al., Biol. Blood Marrow Transplant., 2:100-104 (1996); Walters et al., Blood, 95:1918-1924 (2000)). The lack of matched donors compounds the problem of transplant-mediated toxicity, due to the aggressive regimens used to gain allo-engraftment. Thus, for widespread transplantation to be successful, methods for the generation of allo-chimerism that have low levels of morbidity and mortality are needed. The clinical transplant experience with sickle cell disease suggests that a cure can be achieved even without total replacement of recipient stem cells (Walters et al., Blood, 95:1918-1924 (2000); Vermylen et al., Bone Marrow Transplant., 22:1-6 (1998); Krishnamurti et al., New. Engl. J. Med., 344:68 (2001)). Walters et al. reported that, 4/50 patients treated with conventional myeloablative preconditioning unintentionally developed mixed donor/recipient hematopoiesis (Walters et al., Blood, 95:1918-1924 (2000)). Importantly, those patients with stable mixed chimerism developed no further sickle-related complications. Given these results, as well as similar outcomes in other disease models, there is expanding interest in protocols that are non-myeloablative, and that intentionally produce stable mixed chimerism (Champlin et al., Curr. Opin. Oncol., 11:87-95 (1999); Spitzer et al., Biol. Blood Marrow Transplant., 6:309-320 (2000); Craddock, Curr. Opin. Hematol., 6:383-387 (1999)). One problem to overcome is one of tolerance, as there must be a co-existence of both host and donor cells in order for stable mixed chimerism to be achieved. While the initial protocols used relatively nonspecific immunosuppressive agents to induce transplantation tolerance, recent murine studies have focused on blocking T cell activation pathways as a targeted approach for developing donor-specific tolerance and long-term mixed chimerism (Tomita et al., J. Immunol., 153:1087-1098 (1994); Sykes et al., Nature Medicine, 3:783-787 (1997); Wekerle et al., J. Exp. Med., 187:2037-2044 (1998); Durham et al., J. Immunol., 165:1-4 (2000); Salomon et al., Annu. Rev. Immunol., 19:225-252 (2001)). These studies have shown that disruption of the T cell costimulation signal mediated by the CD28/B7 or CD40/CD40L pathways at the time of bone marrow transplantation can lead to anergy of donor-reactive host T cells and produce long-term tolerance to the graft.

Several features should be considered in the design of a tolerance induction strategy. First, the strategy should provide means to control the existing population of donor-specific T cells in the recipient subject's immune system. Second, the strategy should provide means to control donor-specific T cells that may be generated in the future. Third, the strategy must protect the allograft from irreversible immunologic injury during tolerance induction and maintenance.

### SUMMARY OF INVENTION

Accordingly, the present invention provides methods for establishing titratable degrees of hematopoietic chimerism dependent on the intended application. For example, lower levels of chimerism for the induction of organ transplant tolerance and higher levels of chimerism for the treatment of hemoglobinopathies, such as sickle cell diseases or the various thalassemias. Preferably, chimerism is established without myeloablative conditioning or treatment. However, myeloablative conditioning or treatment can be provided before, during, or after the methods of the invention as a supplemental treatment.

In one embodiment, a method of establishing mixed hematopoietic chimerism comprises administering T cell depleted bone marrow cells to a subject, and administering an alkylating agent to the subject. This method can further comprise an additional step or steps of administering an immunosuppressive agent, and/or administering an additional dose or doses of T cell depleted bone marrow cells, to the subject. The foregoing methods are also useful for treating hemoglobinopathies, and/or inhibiting rejection of an organ or tissue transplant in the subject, as described herein.

In another embodiment, the invention provides methods for treating hemoglobinopathies in a subject. In a preferred embodiment, the methods comprise the steps of administering T cell depleted bone marrow cells and an immunosuppressive agent to a subject, and administering an alkylating agent to the subject. The methods can also include another step of administering a second dose of T cell depleted bone marrow cells and/or the immunosuppressive agent to the subject. These methods may also be practiced by one or more additional steps of administering additional doses of the immunosuppresive agent and/or the alkylating agent to the subject. In certain embodiments, the hemoglobinopathy is beta-thalassemia or sickle cell disease.

In another embodiment, methods of inhibiting rejection of an organ or tissue transplant are provided comprising administering an alkylating agent and T cell depleted bone marrow cells to a subject receiving the transplant. The alkylating agent can be administered to the recipient subject within the twenty-four hours preceding the transplant.

The invention further provides methods for reducing rejection of an organ transplant in a subject comprising the steps of administering to a subject (1) a first dose of T cell depleted bone marrow cells (2) an immunosuppressive agent, an alkylating agent, and a second dose of T cell depleted bone marrow cells and an immunosuppressive agent. The alkylating agent can be administered before, during, or after the bone marrow has been administered. Further the second dose of bone marrow can be administered before, during, or after administration of the alkylating agent. Additionally, the methods can include an additional step or steps of administering an immunosuppressive agent and/or alkylating agent to the subject.

As discussed herein, the immunosuppressive agents useful in the foregoing methods include compositions having molecules that preferably interfere with the interaction of T and B cell costimulatory molecules. In particular, preferred immunosuppressive agents include molecules that interfere with the binding of CD28 antigen to B7 antigen, and molecules that interfere with the binding of gp39 antigen to CD40 antigen. Examples of such agents include soluble forms of CTLA4, (e.g., CTLA4-Ig), soluble forms of CD28 (e.g., CD28-Ig), anti-B7 mAbs, and anti-gp39 (anti-CD40L) mAbs.

In addition, as discussed herein, the preferred alkylating agent used in the foregoing methods is an alkyl sulfonate. More preferably, the alkyl sulfonate is busulfan.

### BRIEF DESCRIPTION OF THE FIGURES

FIG. 1A illustrates percent chimerism as a function of time. Percent chimerism is measured as the percent of CD45.1⁺ cells present in peripheral blood following syngeneic bone marrow transplant with busulfan, as described in Example 1, infra.

FIG. 1B illustrates percent chimerism as a function of time. Percent chimerism is measured as the percent of CD45.1⁺ cells present in peripheral blood following allogeneic bone marrow transplant with busulfan, as described in Example 1, infra.

FIG. 1C depicts the percentage of donor cells (H-2^{d+}) present in peripheral blood from groups that received either T cell-depleted bone marrow (TDBM), costimulation blockade (CB), busulfan (Bus), or bone marrow and costimulation blockade (without busulfan), as described in Example 1, infra. The presence of CD4⁺ and B220⁺ donor cells in peripheral blood demonstrates that without busulfan, animals fail to become chimeric.

FIG. 1D depicts results of bone marrow dose titration with busulfan, as described in Example 1, infra.

FIG. 2 illustrates the effects on peripheral C57BL/6 white blood cells (WBC's) (×10³/mm³ in response to treatment with busulfan (20 mg/kg, day-1), T cell-depleted bone marrow (Balb/c), and costimulation blockade (closed squares), and in response to 3Gy irradiation (day 0), T cell-depleted bone marrow (Balb/c) and costimulation blockade (450 μg MR1 day 0 and 500 μg CTLA4-Ig day 2) (closed triangles), as described in Example 1, infra. The number of WBC's is shown as a function of time.

FIG. 3A is an image of a cellulose acetate gel displaying murine hemoglobin components, as described in Example 2, infra.

FIG. 3B shows the percent of reticulocytes as a function of time, as described in Example 2, infra.

FIG. 4A depicts the percent of animals receiving a skin graft that survived as shown as a function of time, as described in Example 3, infra.

FIG. 4B illustrates the percent of animals that survive after receiving a third party skin graft, or a secondary donor skin graft, as a function of time, as described in Example 3, infra.

FIG. 5A depicts the number of IFNγ producing cells as a function of treatment protocol, as described in Examples 4 & 5, infra. The number of cells are measured at 10 days after skin graft and >100 days after skin graft.

FIG. 5B illustrates the percent of specific lysis as a function of the effector to target cell (E:T) ratio, as described in Examples 4 & 5, infra.

FIG. 5C shows the percent specific lysis as a function of E:T ratio, as described in Examples 4 & 5, infra. Data were obtained from animals receiving secondary donor skin grafts.

FIG. 5D depicts the percent of surviving animals that have received a skin graft as a function of time, as described in Examples 4 & 5, infra.

FIG. 6A shows the percent of CD4⁺ T cells versus the expression of various T cell markers, as described in Example 6, infra.

FIG. 6B depicts histograms of representative animals demonstrating that CD8⁺ T cells from recipients treated with T cell-depleted bone marrow and costimulation blockade (without busulfan) undergo maximal division (up to 8), comparable to naive B6 T cells in the presence of donor tissues, as described in Example 6, infra. Tolerant animals, however, show no proliferation to donor but a normal proliferative response to third party grafts (C3H, H-2^{k}).

FIG. 7A depicts the percent of H2K^{d} positive cells as a function of time in subjects treated with busulfan and costimulation blockade, as described in Example 7, infra.

FIG. 7B shows the percent of donor engraftment as a function of tissue or organ, as described in Example 7, infra.

FIG. 8 is a hemoglobin electrophoretic gel illustrating replacement of the peripheral blood with donor hemoglobin, as described in Example 7, infra.

FIG. 9 is a hemoglobin electrophoretic gel illustrating establishment of red blood cell chimerism in subjects that only received costimulation blockade (i.e., not busulfan), as described in Example 7, infra.

FIG. 10A depicts the number of Vβ5 positive cells (as a percent of CD4 positive T cells) for non-engrafted, engrafted, and BALB/c mice, as described in Example 7, infra.

FIG. 10B illustrates T cell proliferative capacity against donor and third party grafts using an in vivo allo-proliferation model with CFSE-labeled T cells from engrafted and non-engrafted animals, as described in Example 7, infra.

FIGS. 11A and 11B are peripheral blood smears from an untreated animal (A) and an engrafted animal (B), as described in Example 7, infra.

FIG. 11C illustrates that hematological parameters are normalized in engrafted mice, as described in Example 7, infra.

FIG. 11D demonstrates that red blood cells of engrafted mice have normal half-lives, as described in Example 7, infra.

FIG. 11E illustrates that the engrafted red blood cell population is healthy, as described in Example 7, infra.

FIG. 12A depicts spleen weight, expressed as a percent of total body weight in C57BL/6 control, untreated sickle, and engrafted animals, as described in Example 7, infra.

FIG. 12B demonstrates that the balance of hematopoiesis in the spleen was normalized in engrafted mice, as described in Example 7, infra.

FIGS. 12C and 12D are histological sections of the spleen from an untreated mouse (C) and from an engrafted mouse (D), as described in Example 7, infra.

FIGS. 13A and 13B are histological sections of the kidney from an untreated mouse (A) and from an engrafted mouse (B), as described in Example 7, infra.

FIG. 14 shows the nucleotide and amino acid sequences of L104Eig (SEQ ID NOs.: 1-2), as described in Example 8, infra.

FIG. 15 shows the nucleotide and amino acid sequences of L104EA29YIg (SEQ ID NOs.: 3-4), as described in Example 8, infra.

FIG. 16 shows the nucleotide and amino acid sequence of L104EA29LIg (SEQ ID NOs.: 5-6), as described in Example 8, infra.

FIG. 17 shows the nucleotide and amino acid sequences of L104EA29TIg (SEQ ID NOs.: 7-8), as described in Example 8, infra.

FIG. 18 shows the nucleotide and amino acid sequences of L104EA29Wig (SEQ ID NOs.: 9-10), as described in Example 8, infra.

FIG. 19 shows the nucleotide and amino acid sequences of CTLA4 receptor (SEQ ID NOs.: 11-12)

FIG. 20 shows the nucleotide and amino acid sequences of CTLA4Ig (SEQ ID NOs.: 13-14).

FIG. 21 shows a SDS gel (FIG. 21A) for CTLA4Ig (lane 1), L104EIg (lane 2), and L104EA29YIg (lane 3A); and size exclusion chromatographs of CTLA4Ig (FIG. 21B) and L104EA29YIg (FIG. 21C).

FIG. 22 (left and right depictions) shows a ribbon diagram of the CTLA4 extracellular Ig V-like fold generated from the solution structure determined by NMR spectroscopy. FIG. 22 (right depiction) shows an expanded view of the CDR-1 (S25-R33) region and the MYPPPY (SEQ ID NO.: 15) region indicating the location and side-chain orientation of the avidity enhancing mutations, L104 and A29.

FIGS. 23A & 23B show FACS assays showing binding of L104EA29YIg, L104EIg, and CTLA4Ig to human CD80- or CD86-transfected CHO cells as described in Example 9, infra.

FIGS. 24A & 24B are graphs showing inhibition of proliferation of CD80-positive and CD86-positive CHO cells as described in Example 9, infra.

FIGS. 25A & 25B are graphs showing that L104EA29YIg is more effective than CTLA4Ig at inhibiting proliferation of primary and secondary allostimulated T cells as described in Example 9, infra.

FIGS. 26A-C are graphs illustrating that L104EA29YIg is more effective than CTLA4Ig at inhibiting IL-2 (FIG. 26A), IL-4 (FIG. 26B), and gamma (γ)-interferon (FIG. 26C) cytokine production of allostimulated human T cells as described in Example 9, infra.

FIG. 27 is a graph demonstrating that L104EA29YIg is more effective than CTLA4Ig at inhibiting proliferation of phytohemaglutinin-(PHA) stimulated monkey T cells as described in Example 9, infra.

FIG. 28 is a graph showing the equilibrium binding analysis of L104EA29YIg, L104EIg, and wild-type CTLA4Ig to CD86Ig as described in Example 9, infra.

FIG. 29 is a graph showing that LCMV infection impedes extended allograft survival following treatment with anti-CD40L and anti-CTLA4-Ig, as described in Example 10, infra.

FIG. 30 shows that acute LCMV infection impedes tolerance, mixed chimerism, and deletion of donor-reactive T cells, as described in Example 10, infra. A, B6 mice received a BALB/c skin graft along with BALB/c bone marrow on postoperative days 0 and 6. All groups also received anti-CD40L and CTLA4-Ig on days 0, 2, 4, and 6. Mice were further treated with hematopoietic stem cell selective busulfan on day 5 post transplant. B, Uninfected mice proceeded to develop >60% H-2K^{d+} cells in the peripheral blood in all animals by day 120 posttransplant. Infected mice, with or without depletion of CD8 T cells failed to develop mixed chimerism. CD4 T cell subsets expressing Vβ5 (C) and Vβ11 (D) are deleted in uninfected mice by postoperative day 60. These susets are normally deleted in BALB/c but not B6 mice due to expression of MMTV superantigens in conjunction with I-E by BALB/c cells. Infected mice, with or without depletion of CD8 T cells, fail to delete these T cell subsets. All error bars represent the SEM.

FIG. 31 shows that delayed LCMV infection does not impair (A) tolerance induction or (B) the development of mixed chimerism, as described in Example 10, infra.

FIG. 32 shows that the antiviral T cell response following delayed infection is moderately decreased but epitope hierarchy remains unchanged, as described in Example 10, infra. One group received allogeneic (BALB/c) bone marrow and skin grafts, while another group received syngeneic (B6) bone marrow and skin grafts.

FIG. 33 shows that tetramer-positive LCMV-immune CD8 T cells do not divide in response to alloantigen, as described in Example 10, infra. Recipient BALB/c mice were irradiated. Naïve and LCMV-immune donor splenocytes were enriched for T cells. Cells were labeled with the fluorescent dye CFSE (Molecular Probes) and injected into irradiated recipients. Splenocytes were harvested on day 3 posttransfer and stained for expression of CD8 and tetramers. In the first column, spleenocytes were gated for CD8 expression and the histogram displays CFSE fluorescence. Peaks to the right of the histogram represent highly fluorescent, undivided cells, while successive peaks to the left measure loss of fluorescence with each cell division. Next, splenocytes were gated for undivided (middle column) and highly divided (four to eight divisions, right column) CD8 T cells and assessed for their ability to bind class I tetramers folded into two immunodominant LCMV peptides. Representative samples from three mice per group are shown.

FIG. 34 shows that IFN-γ⁺ LCMV-immune cells do not divide in response to alloantigen, as described in Example 10, infra. Naive and LCMV-immune CSFE-labeled T cells were transferred into irradiated BALB/c recipients and harvested as described in FIG. 33. Splenocytes were incubated in brefeldin A with LCMV-infected or uninfected MC57 fibrosarcoma cells for 5 h. Cells were fixed and permeabilized, stained for expression of CD8 and IFN-γ⁺, and analyzed by flow cytometry. Splenocytes were gated for CD8 expression (left column) and assessed CFSE fluorescence as in FIG. 33. Undivided (middle column) and highly divided (four to eight divisions, right column) CD8 T cells were measured for IFN-γ staining. A representative sample of two separate experiments is shown.

FIG. 35 shows that LCMV stimulates the CD28/CD40-independent generation of alloreactive IFN-γ-producing T cells, as described in Example 10, infra. C3H/HeJ mice either received a BALB/c skin graft (SG) or a skin graft with costimulation blockade (CB). A third group received a skin graft and costimulation blockade concurrent with an LCMV infection, while a fourth group received an LCMV infection without further manipulation. Mouse spleens were harvested on the indicated days, and the frequency of IFN-γ producing cells specific for LCMV or alloantigen was determined using an ELISPLT assay as described in Example 10. Error bars represent the SEM (n=3 for all groups).

FIG. 36 shows that LCMV infection drives the CD28/CD40-independent maturation of dendritic cells, as described in Example 10, infra. B6 mice received either BALB/c bone marrow and costimulation blockade, or the same regimen concurrent with an LCMV infection. Spleens were harvested on day 6 post-transplant. CD11c⁺ dendritic cells were enriched, stained with the indicated Abs, and analyzed by flow cytometry. Histograms represent expression of the indicated molecules among cells gated for CD11c expression. Filled histograms represent mice treated with bone marrow and costimulation blockade, solid lines represent mice receiving a concurrent LCMV infection, and dotted lines are isotype controls. These histograms are representative of two separate experiments.

In order that the invention herein described may be more fully understood, the following description is set forth.

### DETAILED DESCRIPTION OF THE INVENTION

Definitions

All scientific and technical terms used in this application have meanings commonly used in the art unless otherwise specified. As used in this application, the following words or phrases have the meanings specified.

As used herein, “transplant rejection” is defined as the nearly complete, or complete, loss of viable graft tissue from the recipient subject. In the case of skin grafts, “rejection” is defined as the nearly complete, or complete, loss of viable epidermal graft tissue.

As used herein, “mixed hematopoietic chimerism” is defined as the presence of donor and recipient blood progenitor and mature cells (e.g., blood deriving cells) in the absence (or undetectable presence) of an immune response.

As used herein, “costimulatory pathway” is defined as a biochemical pathway resulting from interaction of costimulatory signals on T cells and antigen presenting cells (APCs). Costimulatory signals help determine the magnitude of an immunological response to an antigen. One costimulatory signal is provided by the interaction with T cell receptors CD28 and CTLA4 and B7 molecules on APCs. As used herein, “B7” includes B7-1 (also called CD80), B7-2 (also called CD86), B7-3 (also called CD74), and the B7 family, e.g., a combination of B7-1, B7-2, and/or B7-3. Another example is provided by the interaction of CD40 and gp39 (also called CD154). As used herein, gp39 is also referred to as CD154 or CD40L. The terms gp39, CD154, and CD40L are used interchangeably in this application.

As used herein, “costimulatory blockade” is defined as a protocol of administering to a subject, one or more agents that interfere or block a costimulatory pathway, as described above. Examples of agents that interfere with the costimulatory blockade include, but are not limited to, soluble CTLA4, soluble CD28, anti-B7 monoclonal antibodies (mAbs), soluble CD40, and anti-gp39 mAbs. These agents are also considered “immunosuppressive agents”. “Immunosuppressive agent” is defined as a composition having one or more types of molecules that prevent the occurrence of an immune response, or weaken a subject's immune system.

As used herein, “monoclonal antibodies directed against gp39” or “anti-gp39 mAbs” or “anti-CD154 mAb” or “anti-CD40L mAbs” include any antibody molecule, fragment thereof, or recombinant binding protein that recognizes and binds gp39, or fragment thereof.

As used herein, “a soluble ligand which recognizes and binds B7 antigen” includes CTLA4-Ig, CD28-Ig or other soluble forms of CTLA4 and CD28, including recombinant and/or mutant CTLA4 and CD28, and includes any antibody molecule, fragment thereof or recombinant binding protein that recognizes and binds a B7 antigen. These agents are also considered ligands that interfere with the binding of CD28 to B7 and gp39 to CD40. As used herein, “T cell depleted bone marrow” is defined as bone marrow removed from bone and that has been exposed to an anti-T cell protocol. An anti-T cell protocol is defined as a procedure for removing T cells from bone marrow. Methods of selectively removing T cells are well known in the art. An example of an anti-T cell protocol is exposing bone marrow to T cell specific antibodies, such as anti-CD3, anti-CD4, anti-CD5, anti-CD8, and anti-CD90 monoclonal antibodies, wherein the antibodies are cytotoxic to the T cells. Alternatively, the antibodies can be coupled to magnetic particles to permit removal of T cells from bone marrow using magnetic fields. Another example of an anti-T cell protocol is exposing bone marrow T cells to anti-lymphocyte serum or anti-thymocyte globulin.

As used herein, “tolerizing dose of T cell depleted bone marrow” is defined as an initial dose of T cell depleted bone marrow that is administered to a subject for the purpose of inactivating potential donor reactive T cells.

As used herein, “engrafting dose of T cell depleted bone marrow” is defined as a subsequent dose of T cell depleted bone marrow that is administered to a subject for the purpose of establishing mixed hematopoietic chimerism. The engrafting dose of T cell depleted bone marrow will accordingly be administered after the tolerizing dose of T cell depleted bone marrow.

As used herein, “tissue transplant” is defined as a tissue of all, or part of, an organ that is transplanted to a recipient subject. In certain embodiments, the tissue is from one or more solid organs. Examples of tissues or organs include, but are not limited to, skin, heart, lung, pancreas, kidney, liver, bone marrow, pancreatic islet cells, cell suspensions, and genetically modified cells. The tissue can be removed from a donor subject, or can be grown in vitro. The transplant can be an autograft, isograft, allograft, or xenograft, or a combination thereof.

As used herein, “administer” or “administering” means provided by any means including intravenous (i.v.) administration, intra-peritoneal (i.p.) administration, intramuscular (i.m.) administration, subcutaneous administration, oral administration, administration as a suppository, or as a topical contact, or the implantation of a slow-release device such as a miniosmotic pump, to the subject.

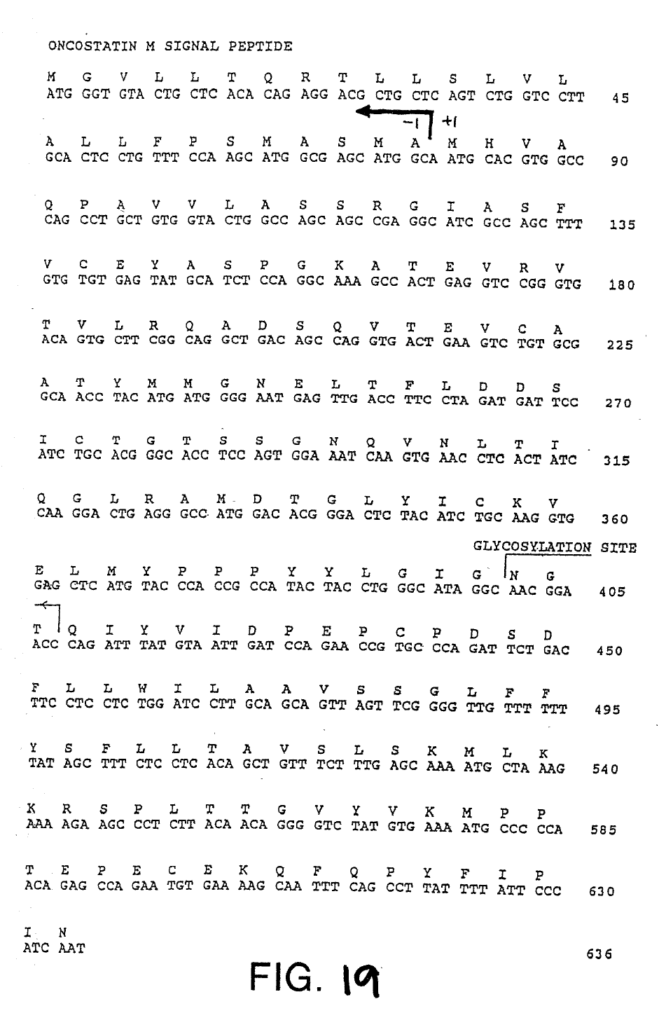

As used herein “wild type CTLA4” or “non-mutated CTLA4” has the amino acid sequence of naturally occurring, full length CTLA4 as shown in FIG. 19 (also as described U.S. Pat. Nos. 5,434,131, 5,844,095, 5,851,795), or any portion or derivative thereof, that recognizes and binds a B7 or interferes with a B7 so that it blocks binding to CD28 and/or CTLA4 (e.g., endogenous CD28 and/or CTLA4). In particular embodiments, the extracellular domain of wild type CTLA4 begins with methionine at position +1 and ends at aspartic acid at position +124, or the extracellular domain of wild type CTLA4 begins with alanine at position −1 and ends at aspartic acid at position +124. Wild type CTLA4 is a cell surface protein, having an N-terminal extracellular domain, a transmembrane domain, and a C-terminal cytoplasmic domain. The extracellular domain binds to target molecules, such as a B7 molecule. In a cell, the naturally occurring, wild type CTLA4 protein is translated as an immature polypeptide, which includes a signal peptide at the N-terminal end. The immature polypeptide undergoes post-translational processing, which includes cleavage and removal of the signal peptide to generate a CTLA4 cleavage product having a newly generated N-terminal end that differs from the N-terminal end in the immature form. One skilled in the art will appreciate that additional post-translational processing may occur, which removes one or more of the amino acids from the newly generated N-terminal end of the CTLA4 cleavage product. Alternatively, the signal peptide may not be removed completely, generating molecules that begin before the common starting amino acid methionine. Thus, the mature CTLA4 protein may start at methionine at position +1 or alanine at position −1. The mature form of the CTLA4 molecule includes the extracellular domain or any portion thereof, which binds to B7.

As used herein, a “CTLA4 mutant molecule” means wildtype CTLA4 as shown in FIG. 19 or any portion or derivative thereof, that has a mutation or multiple mutations (preferably in the extracellular domain of wildtype CTLA4). A CTLA4 mutant molecule has a sequence that it is similar but not identical to the sequence of wild type CTLA4 molecule, but still binds a B7. The mutations may include one or more amino acid residues substituted with an amino acid having conservative (e.g., substitute a leucine with an isoleucine) or non-conservative (e.g., substitute a glycine with a tryptophan) structure or chemical properties, amino acid deletions, additions, frameshifts, or truncations. CTLA4 mutant molecules may include a non-CTLA4 molecule therein or attached thereto. The mutant molecules may be soluble (i.e., circulating) or bound to a cell surface. Additional CTLA4 mutant molecules include those described in U.S. patent application Ser. Nos. 09/865,321, 60/214,065 and 60/287,576; in U.S. Pat. Nos. 6,090,914 5,844,095 and 5,773,253; and as described by Peach, R. J., et al., in J Exp Med 180:2049-2058 (1994)). CTLA4 mutant molecules can be made synthetically or recombinantly.

“CTLA4Ig” is a soluble fusion protein comprising an extracellular domain of wildtype CTLA4 joined to an Ig tail, or a portion thereof that binds a B7. A particular embodiment comprises the extracellular domain of wild type CTLA4 (as shown in FIG. 19) starting at methionine at position +1 and ending at aspartic acid at position +124; or starting at alanine at position −1 to aspartic acid at position +124; a junction amino acid residue glutamine at position +125; and an immunoglobulin portion encompassing glutamic acid at position +126 through lysine at position +357 (DNA encoding CTLA4Ig was deposited on May 31, 1991 with the American Type Culture Collection (ATCC), 10801 University Blvd., Manassas, Va. 20110-2209 under the provisions of the Budapest Treaty, and has been accorded ATCC accession number ATCC 68629; Linsley, P., et al., 1994 Immunity 1:793-80). CTLA4Ig-24, a Chinese Hamster Ovary (CHO) cell line expressing CTLA4Ig was deposited on May 31, 1991 with ATCC identification number CRL-10762). The soluble CTLA4Ig molecules used in the methods and/or kits of the invention may or may not include a signal (leader) peptide sequence. Typically, in the methods and/or kits of the invention, the molecules do not include a signal peptide sequence.

“L104EA29YIg” is a fusion protein that is a soluble CTLA4 mutant molecule comprising an extracellular domain of wildtype CTLA4 with amino acid changes A29Y (a tyrosine amino acid residue substituting for an alanine at position 29) and L104E (a glutamic acid amino acid residue substituting for a leucine at position +104), or a portion thereof that binds a B7 molecule, joined to an Ig tail (included in FIG. 15; DNA encoding L104EA29YIg was deposited on Jun. 20, 2000 with ATCC number PTA-2104; copending in U.S. patent application Ser. Nos. 09/579,927, 60/287,576 and 60/214,065, incorporated by reference herein). The soluble L104EA29YIg molecules used in the methods and/or kits of the invention may or may not include a signal (leader) peptide sequence. Typically, in the methods and/or kits of the invention, the molecules do not include a signal peptide sequence.

As used herein, “soluble” refers to any molecule, or fragments and derivatives thereof, not bound or attached to a cell, i.e., circulating. For example, CTLA4, B7 or CD28 can be made soluble by attaching an immunoglobulin (Ig) moiety to the extracellular domain of CTLA4, B7 or CD28, respectively. Alternatively, a molecule such as CTLA4 can be rendered soluble by removing its transmembrane domain. Typically, the soluble molecules used in the methods of the invention do not include a signal (or leader) sequence.

As used herein, “soluble CTLA4 molecules” means non-cell-surface-bound (i.e. circulating) CTLA4 molecules (wildtype or mutant) or any functional portion of a CTLA4 molecule that binds B7 including, but not limited to: CTLA4Ig fusion proteins (e.g. ATCC 68629), wherein the extracellular domain of CTLA4 is fused to an immunoglobulin (Ig) moiety rendering the fusion molecule soluble, or fragments and derivatives thereof, proteins with the extracellular domain of CTLA4 fused or joined with a portion of a biologically active or chemically active protein such as the papillomavirus E7 gene product (CTLA4-E7), melanoma-associated antigen p97 (CTLA4-p97) or HIV env protein (CTLA4-env gp120), or fragments and derivatives thereof; hybrid (chimeric) fusion proteins such as CD28/CTLA4Ig, or fragments and derivatives thereof; CTLA4 molecules with the transmembrane domain removed to render the protein soluble (Oaks, M. K., et al., 2000 Cellular Immunology 201:144-153), or fragments and derivatives thereof. “Soluble CTLA4 molecules” also include fragments, portions or derivatives thereof, and soluble CTLA4 mutant molecules, having CTLA4 binding activity. The soluble CTLA4 molecules used in the methods of the invention may or may not include a signal (leader) peptide sequence. Typically, in the methods of the invention, the molecules do not include a signal peptide sequence.

As used herein “the extracellular domain of CTLA4” is a portion of CTLA4 that recognizes and binds CTLA4 ligands, such as B7 molecules. For example, an extracellular domain of CTLA4 comprises methionine at position +1 to aspartic acid at position +124 (FIG. 19). Alternatively, an extracellular domain of CTLA4 comprises alanine at position −1 to aspartic acid at position +124 (FIG. 19). The extracellular domain includes fragments or derivatives of CTLA4 that bind a B7 molecule. The extracellular domain of CTLA4 as shown in FIG. 19 may also include mutations that change the binding avidity of the CTLA4 molecule for a B7 molecule.

As used herein, the term “mutation” means a change in the nucleotide or amino acid sequence of a wildtype molecule, for example, a change in the DNA and/or amino acid sequences of the wild-type CTLA4 extracellular domain. A mutation in DNA may change a codon leading to a change in the amino acid sequence. A DNA change may include substitutions, deletions, insertions, alternative splicing, or truncations. An amino acid change may include substitutions, deletions, insertions, additions, truncations, or processing or cleavage errors of the protein. Alternatively, mutations in a nucleotide sequence may result in a silent mutation in the amino acid sequence as is well understood in the art. In that regard, certain nucleotide codons encode the same amino acid.

Examples include nucleotide codons CGU, CGG, CGC, and CGA encoding the amino acid, arginine (R); or codons GAU, and GAC encoding the amino acid, aspartic acid (D). Thus, a protein can be encoded by one or more nucleic acid molecules that differ in their specific nucleotide sequence, but still encode protein molecules having identical sequences. The amino acid coding sequence is as follows:

| | | One | |
|---|---|---|---|
| | | Letter | |
| | | Sym- | |
| Amino Acid | Symbol | bol | Codons |
| Alanine | Ala | A | GCU, GCC, GCA, GCG |
| Cysteine | Cys | C | UGU, UGC |
| Aspartic Acid | Asp | D | GAU, GAC |
| Glutamic Acid | Glu | E | GAA, GAG |
| Phenylalanine | Phe | F | UUU, UUC |
| Glycine | Gly | G | GGU, GGC, GGA, GGG |
| Histidine | His | H | CAU, CAC |
| Isoleucine | Ile | I | AUU, AUC, AUA |
| Lysine | Lys | K | AAA, AAG |
| Leucine | Leu | L | UUA, UUG, CUU, CUC, CUA, CUG |
| Methionine | Met | M | AUG |
| Asparagine | Asn | N | AAU, AAC |
| Proline | Pro | P | CCU, CCC, CCA, CCG |
| Glutamine | Gln | Q | CAA, CAG |
| Arginine | Arg | R | CGU, CGC, CGA, CGG, AGA, AGG |
| Serine | Ser | S | UCU, UCC, UCA, UCG, AGU, AGC |
| Threonine | Thr | T | ACU, ACC, ACA, ACG |
| Valine | Val | V | GUU, GUC, GUA, GUG |
| Tryptophan | Trp | W | UGG |
| Tyrosine | Tyr | Y | UAU, UAC |

The mutant molecule may have one or more mutations.

As used herein, a “non-CTLA4 protein sequence” or “non-CTLA4 molecule” means any protein molecule that does not bind B7 and does not interfere with the binding of CTLA4 to its target. An example includes, but is not limited to, an immunoglobulin (Ig) constant region or portion thereof. Preferably, the Ig constant region is a human or monkey Ig constant region, e.g., human C(gamma)l, including the hinge, CH2 and CH3 regions. The Ig constant region can be mutated to reduce its effector functions (U.S. Pat. Nos. 5,637,481, 5,844,095 and 5,434,131).

As used herein, a “fragment” or “portion” is any part or segment of a CTLA4 molecule, preferably the extracellular domain of CTLA4 or a part or segment thereof, that recognizes and binds its target, e.g., a B7 molecule.

As used herein, “B7” refers to the B7 family of molecules including, but not limited to, B7-1 (CD80), B7-2 (CD86) and B7-3 that may recognize and bind CTLA4 and/or CD28.

As used herein, “B7-positive cells” are any cells with one or more types of B7 molecules expressed on the cell surface.

As used herein, a “derivative” is a molecule that shares sequence homology and activity of its parent molecule. For example, a derivative of CTLA4 includes a soluble CTLA4 molecule having an amino acid sequence at least 70% similar to the extracellular domain of wildtype CTLA4, and which recognizes and binds B7 e.g. CTLA4Ig or soluble CTLA4 mutant molecule L104EA29YIg.

As used herein, to “block” or “inhibit” a receptor, signal or molecule means to interfere with the activation of the receptor, signal or molecule, as detected by an art-recognized test. For example, blockage of a cell-mediated immune response can be detected by determining reduction of transplant rejection or decreasing symptoms associated with hemoglobinopathies. Blockage or inhibition may be partial or total.

As used herein, “blocking B7 interaction” means to interfere with the binding of B7 to its ligands, such as CD28 and/or CTLA4, thereby obstructing T-cell and B7-positive cell interactions. Examples of agents that block B7 interactions include, but are not limited to, molecules such as an antibody (or portion or derivative thereof) that recognizes and binds to the any of CTLA4, CD28 or B7 molecules (e.g. B7-1, B7-2); a soluble form (or portion or derivative thereof) of the molecules such as soluble CTLA4; a peptide fragment or other small molecule designed to interfere with the cell signal through the CTLA4/CD28/1B7-mediated interaction. In a preferred embodiment, the blocking agent is a soluble CTLA4 molecule, such as CTLA4Ig (ATCC 68629) or L104EA29YIg (ATCC PTA-2104), a soluble CD28 molecule such as CD28Ig (ATCC 68628), a soluble B7 molecule such as B7Ig (ATCC 68627), an anti-B7 monoclonal antibody (e.g. ATCC HB-253, ATCC CRL-2223, ATCC CRL-2226, ATCC HB-301, ATCC HB-11341 and monoclonal antibodies as described in by Anderson et al in U.S. Pat. No. 6,113,898 or Yokochi et al., 1982. J. Immun., 128(2):823-827), an anti-CTLA4 monoclonal antibody (e.g. ATCC HB-304, and monoclonal antibodies as described in references 82-83) and/or an anti-CD28 monoclonal antibody (e.g. ATCC HB 11944 and mAb 9.3 as described by Hansen (Hansen et al., 1980. Immunogenetics 10:247-260) or Martin (Martin et al., 1984. J. Clin. Immun., 4(1):18-22)).

As used herein, “immune system disease” means any disease mediated by T-cell interactions with B7-positive cells including, but not limited to, autoimmune diseases, graft related disorders and immunoproliferative diseases. Examples of immune system diseases include graft versus host disease (GVHD) (e.g., such as may result from bone marrow transplantation, or in the induction of tolerance), immune disorders associated with graft transplantation rejection, chronic rejection, and tissue or cell allo- or xenografts, including solid organs, skin, islets, muscles, hepatocytes, neurons. Examples of immunoproliferative diseases include, but are not limited to, psoriasis, T-cell lymphoma, T-cell acute lymphoblastic leukemia, testicular angiocentric T-cell lymphoma, benign lymphocytic angiitis, lupus (e.g. lupus erythematosus, lupus nephritis), Hashimoto's thyroiditis, primary myxedema, Graves' disease, pernicious anemia, autoimmune atrophic gastritis, Addison's disease, diabetes (e.g. insulin dependent diabetes mellitis, type I diabetes mellitis, type II diabetes mellitis), good pasture's syndrome, myasthenia gravis, pemphigus, Crohn's disease, sympathetic ophthalmia, autoimmune uveitis, multiple sclerosis, autoimmune hemolytic anemia, idiopathic thrombocytopenia, primary biliary cirrhosis, chronic action hepatitis, ulceratis colitis, Sjogren's syndrome, rheumatic diseases (e.g. rheumatoid arthritis), polymyositis, scleroderma, and mixed connective tissue disease.

In order that the invention herein described may be more fully understood the following description is set forth.

Methods

The invention disclosed herein provides methods for establishing mixed hematopoietic chimerism in subjects. The subjects include but are not limited to human, monkey, pig, horse, fish, dog, cat and cow. Hematopoietic chimerism may be useful to inhibit an immune response, e.g., inhibit rejection of a transplant, e.g., a tissue or solid organ transplant, and/or may be useful for treating hemoglobinopathies, such as sickle cell diseases and thalassemias. As indicated herein, the organ or tissue transplant can be from any type of organ or tissue amenable to transplantation. By way of example, and not limitation, tissue can be selected from organs including skin, bone marrow, heart, lung, kidney, liver, pancreas, pancreatic islet cells, cell suspensions and genetically modified cells. In one embodiment, the tissue transplant is skin. The tissue can be removed from a donor subject, or can be grown in vitro. The transplant can be an autograft, isograft, allograft, or xenograft, or a combination thereof.

In one embodiment, the invention provides methods for treating an immune system disorder and/or hemoglobinopathies comprising administering an alkylating agent and T cell depleted bone marrow with or without an immunosuppressive agent.

In accordance with the practice of the invention, the method further comprises administering one or more doses of T cell depleted bone marrow cells (tolerizing and/or engrafting dose) to the subject. Also, in accordance with the practice of the invention, the method comprises administering one or more doses of the alkylating agent to the subject. In addition, the method can comprise administering one or more immunosuppressive agents to the subject in a single or multiple administration time points.

In one embodiment, a first dose of T cell depleted bone marrow (tolerizing dose) and the immunosuppressive agent are administered at approximately the same time as the organ transplant. Preferably, the bone marrow and immunosuppressive agent are administered before administration of busulfan. The method may also comprise an additional step or steps of administering at least one type of immunosuppressive agent after administration of busulfan. In addition, the methods can further comprise administering a second dose of T cell depleted bone marrow (engrafting dose) to the subject.

As indicated herein, the alkylating agent is preferably an anti-proliferative agent (e.g., an agent that inhibits cellular proliferation). One example of a preferred alkylating agent is an alkylsulfonate, e.g., busulfan. Other examples of alkylsulfonates include, alkyl p-toluenesulfonates, alkyltrifluoromethanesulfonates, p-bromophenylsulfonates, alkylarylsulfonates, and others. Other examples of alkylating agents include, but are not limited to, nitrogen mustards (mechlorethamines, chlorambucil, melphalan, uracil mustard), oxazaposporines (cyclosphosphamide, perfosfamide, trophosphamide), and nitrosoureas.

Although the preferred embodiments of the invention use the alkylsulfonate, busulfan, as an anti-proliferative agent, other embodiments of the invention may be practiced with other anti-proliferative, chemotherapeutic agents. In certain embodiments, alkylating chemotherapeutic agents will be particularly useful. Examples of other alkylating chemotherapeutic agents include, but are not limited to, carmustine, chlorambucil, cisplatin, lomustine, cyclophosphamide, melphalan, mechlorethamine, procarbazine, thiotepa, uracil mustard, triethylenemelamine, pipobroman, streptozocin, ifosfamide, dacarbazine, carboplatin, and hexamethylmelamine.

Administration of the alkylating agent, as well as other agents, to the subject can be accomplished in many different ways. For example, the alkylating agent can be administered intravenously, intramuscularly, or intra-peritoneally. Alternatively, the agent may be administered orally or subcutaneously. Some methods for administering busulfan are disclosed in U.S. Pat. Nos. 5,430,057 and 5,559,148. Other methods of administration will be recognized by those skilled in the art. Similarly, T cell depleted bone marrow can be administered in many different ways as known by persons skilled in the art. One example is by intravenous infusion. In certain embodiments, the alkylating agent can be administered within twenty-four hours prior to the administration of T cell depleted bone marrow.

Furthermore, the amount of the alkylating agent and T cell depleted bone marrow may be determined by routine experimentation and optimized empirically. Dosage of a therapeutic agent or immunosuppressive agent is dependent upon many factors including, but not limited to, the type of subject (i.e. the species), the agent used (e.g. busulfan, or soluble CTLA4, or anti-gp39 mAb), location of the antigenic challenge, the type of tissue affected, the type of disease being treated, the severity of the disease, a subject's health and response to the treatment with the agents. Accordingly, dosages of the agents can vary depending on each subject, agent and the mode of administration. As described herein, busulfan doses can be titrated to determine the optimal dosage required to achieve the desired effects. For example, busulfan may be administered in an amount between 0.1 to 20 mg/kg weight of the subject, e.g., 4 mg/kg, 8-16 mg/kg, 4-16 mg/kg (Slavin, S. et al., Blood, 91:756-763 (1998)1 Lucarelli et al., supra). Similarly, the amount of T cell depleted bone marrow can be titrated during routine experimentation to determine the amount sufficient to achieve the desired effects.

In some embodiments, the alkylating agent, e.g., busulfan, is administered before the transplant, e.g., tissue or solid organ transplant. Particular embodiments include administering the busulfan within a day, within twelve hours, or within six hours of the solid organ transplant. However, the busulfan can be administered earlier so long as the resulting effects of the busulfan are still achieved in connection with the organ or tissue transplant. In alternative embodiments, it may be desired to administer busulfan after the organ transplant.

Administration of the alkylating agent and/or T cell depleted bone marrow can occur at approximately the same time as the subject receives the solid organ transplant. Administration of the alkylating agent or bone marrow at approximately the same time indicates that the alkylating agent or bone marrow is administered to the subject as part of the preparation for the procedures for administering the organ or tissue transplant. It is not required that the alkylating agent or bone marrow be administered at exactly the same time (i.e., within minutes) as the organ transplant. As persons skilled in the art will appreciate, the timing of the administration of the compositions may vary. For example, the administration of T cell depleted bone marrow cells can occur prior to, subsequently to, or concurrently with, the administration of busulfan. Likewise, the timing of the administration can vary with respect to the administration of immunosuppressive agents or the timing of the organ transplant.

As disclosed herein, preferred immunosuppressive agents are agents that inhibit an immune response. More preferably, the agents reduce or prevent T cell proliferation. Some agents may inhibit T cell proliferation by inhibiting interaction of T cells with other antigen presenting cells. One example of an antigen presenting cell is a B cell. Examples of agents that interfere with T cell interactions with antigen presenting cells, and thereby inhibit T cell proliferation, include, but are not limited to, ligands for B7 antigens, ligands for CTLA4 antigen, ligands for CD28 antigen, ligands for T cell receptor (TCR), ligands for gp39 antigens, ligands for CD40 antigens, ligands for CD4, and ligands for CD8. Examples of ligands for B7 antigens include, but are not limited to, soluble CTLA4 (e.g., ATCC 68629, ATCC PTA 2104), soluble CD28 (e.g., ATCC 68628), or monoclonal antibodies that recognize and bind B7 antigens, or fragments thereof (e.g., ATCC HB-253, ATCC CRL-2223, ATCC CRL-2226, ATCC HB-301, ATCC HB-11341; monoclonal antibodies as described in by Anderson et al in U.S. Pat. No. 6,113,898 or Yokochi et al., 1982. J. Immun., 128(2)₈₋₂₃-827). One preferred agent is CTLA4-Ig (ATCC 68629). Other soluble CTLA4 molecules may also be particularly useful, including soluble CTLA4 mutant molecules (ATCC PTA 2104).

Ligands for CTLA4 or CD28 antigens include monoclonal antibodies that recognize and bind CTLA4 (e.g. ATCC HB-304, and monoclonal antibodies as described in Linsley et al, U.S. Pat. No. 6,090,914 and Linsley et al., 1992. J. Ex. Med 176: 1595-1604) and/or CD28 (e.g. ATCC HB 11944 and mAb 9.3 as described by Hansen (Hansen et al., 1980. Immunogenetics 10: 247-260) or Martin (Martin et al., 1984. J. Clin. Immun., 4(1):18-22)), or fragments thereof. Other ligands for CTLA4 or CD28 include soluble B7 molecules, such as B7Ig (e.g., ATCC 68627).

Examples of ligands for gp39 include, but are not limited to, soluble CD40 or monoclonal antibodies that recognize and bind gp39 antigen (e.g. anti-CD40L), or a fragment thereof. One example of gp39 (anti-CD40L) mAb is MR1 (Bioexpress, Lebanon, N.H.). Additional examples of anti-human-gp39 mAbs include but are not limited to ATCC HB 11822, ATCC HB 11816, ATCC HB 11821, ATCC HB 11808, ATCC HB 11823, described in European patent No. EP 807175A2.

Examples of ligands for CD40 include, but are not limited to, soluble gp39 or monoclonal antibodies that recognize and bind CD40 antigen, or a fragment thereof. Persons skilled in the art will readily understand that other agents or ligands can be used to inhibit the interaction of CD28 with B7, and/or gp39 with CD40. Such agents will be selected to be used in the methods of the invention by the known properties of the agents, for example, the agent interferes with the interaction of CTLA4/CD28 with B7, and/or interferes with the interaction of gp39 with CD40. Knowing that an agent interferes with these interactions permits one skilled in the art to readily practice the methods of the invention with these agents based on the disclosure herein.

In addition, other immunosuppresive agents can be used in the methods of the invention. Examples include: cyclosporin, azathioprine, methotrexate, lymphocyte immune globulin, anti-CD3 antibodies, Rho (D) immune globulin, adrenocorticosteroids, sulfasalzine, FK-506. methoxsalen, mycophenolate mofetil (CELLCEPT), horse anti-human thymocyte globulin (ATGAM), humanized anti-TAC (HAT), basiliximab (SIMULECT), rabbit anti-human thymocyte globulin (THYMOGLOBULIN), sirolimus or thalidomide.

In a preferred embodiment, the immunosuppressive agents are coadministered (i.e., they are administered as a combination treatment) to the subject. In a more preferred embodiment, the combination is a combination of a first ligand that interferes with the binding of CD28 antigen to B7 antigen, and a second ligand that interferes with the binding of gp39 antigen (also designated as CD154) to CD40 antigen. As described supra, the first ligand is preferably a soluble CTLA4 molecule, such as CTLA4-Ig. The second ligand is preferably an anti-gp39 mAb (i.e. a monoclonal antibody that recognizes and binds gp39 antigen, or a fragment thereof). One example is MR1.

In one embodiment of the invention, CTLA4Ig and MR1 are administered in combination to block the costimulatory activity of CTLA4/CD28/B7 and gp39/CD40. Additional embodiments can include CTLA4Ig and an anti-human-gp39 mAb. Examples of anti-human-gp39 mAb include but are not limited to ATCC HB 11822, ATCC HB 11816, ATCC HB 11821, ATCC HB 11808, ATCC HB 11823, described in European patent No. EP 807175A2. As indicated herein, this combination is referred to as a “costimulation blockade”. For example, soluble CTLA4 molecules may be administered in an amount between 0.1 to 20.0 mg/kg weight of the subject, preferably between 0.5 to 10.0 mg/kg.

In one method of the invention involving treatment for hemoglobinopathies in a subject, the method can also include an additional step of administering a second dose of T cell depleted bone marrow to the subject. Similarly, the methods can include one or more additional steps of administering additional doses of the immunosuppressive agent to the subject.

In certain embodiments, the hemoglobinopathy is beta-thalassemia. In other embodiments, the hemoglobinopathy is sickle cell disease. Correction of the hemoglobinopathy can be determined in numerous ways. One example is by measuring the amount of hemoglobin bands (e.g., major or minor) in the recipient subject's blood.

In a preferred embodiment of the invention, the methods comprise administering a first dose of T cell depleted bone marrow and concurrently administering a combination of soluble CTLA4 and a gp39 mAb to the subject, subsequently administering additional doses of the soluble CTLA4 and gp39 mAb to the subject, subsequently administering the alkylating agent, to the subject, and administering a second dose of T cell depleted bone marrow to the subject. The foregoing method is particularly useful for establishing hematopoietic chimerism, treating beta thalassemia, and inhibiting rejection of a tissue or organ transplant.

Compositions

The invention provides compositions useful for establishing chimerism in subjects. The compositions will accordingly be useful for inhibiting an immune response, e.g., inhibiting rejection of tissue or organ transplants. The compositions will also be useful for correcting hemoglobinopathies.

As described herein, the compositions preferably comprise an alkylating agent, such as, busulfan, and one or more types of immunosuppressive agents. Additionally, the composition can comprise T cell depleted bone marrow. Preferably, the T cell depleted bone marrow is immunologically matched to the subject to be treated.

In a preferred embodiment, the composition comprises busulfan and/or the combination of soluble CTLA4, and anti-gp39 mAbs. Specific examples include CTLA4Ig and MR1.

The composition of the invention is preferably administered in a pharmaceutically acceptable carrier, as described above. As persons skilled in the art understand, the composition does not require that the specific agents are coadministered. For example, busulfan can be administered separately from the costimulatory blockade, and still act as a composition to be used in the methods described herein. Alternatively, the composition can include busulfan, soluble CTLA4, and anti-gp39 mAbs in a single carrier. Other embodiments are possible.

The invention also encompasses the use of the compositions of the invention together with other pharmaceutical agents to treat immune system diseases and/or hemoglobinopathies. For example, immune diseases or hemoglobinopathies may be treated with molecules of the invention in conjunction with, but not limited to, immunosuppressants listed supra and additionally any one or more of corticosteroids, cyclosporin (Mathiesen 1989 Cancer Lett. 44(2):151-156), prednisone, azathioprine, methotrexate (R. Handschumacher, in: “Drugs Used for Immunosuppression” pages 1264-1276), TNFα blockers or antagonists (New England Journal of Medicine, vol. 340: 253-259, 1999; The Lancet vol. 354: 1932-39, 1999, Annals of Internal Medicine, vol. 130: 478-486), or any other biological agent targeting any inflammatory cytokine, nonsteroidal antiinflammatory drugs/Cox-2 inhibitors, hydroxychloroquine, sulphasalazopryine, gold salts, etanercept, infliximab, rapamycin, mycophenolate mofetil, azathioprine, tacrolismus, basiliximab, cytoxan, interferon beta-la, interferon beta-1b, glatiramer acetate, mitoxantrone hydrochloride, anakinra and/or other biologics.

Additionally, the invention contemplates the use of the compositions of the invention together with anti-viral agents to promote tolerance in a subject with a concomitant viral infection.

Further provided are therapeutic combinations, e.g. a kit, e.g. for use in any method as defined above, comprising a soluble CTLA4 molecule, in free form or in pharmaceutically acceptable salt form, to be used concomitantly or in sequence with at least one pharmaceutical composition comprising an immunosuppressant, immunomodulatory or anti-inflammatory drug, and/or an alkylating agent. The kit may comprise instructions for its administration. The immunosuppressant, immunomodulatory or anti-inflammatory drug can be in free form or in pharmaceutically acceptable salt form. Additionally, the alkylating agent can be in free form or in pharmaceutically acceptable salt form.

Soluble CTLA4 molecules are the preferred ligands that interfere with CTLA4/CD28/B7 interaction. CTLA4 molecules, with mutant or wildtype sequences, may be rendered soluble by deleting the CTLA4 transmembrane segment (Oaks, M. K., et al., 2000 Cellular Immunology 201:144-153).

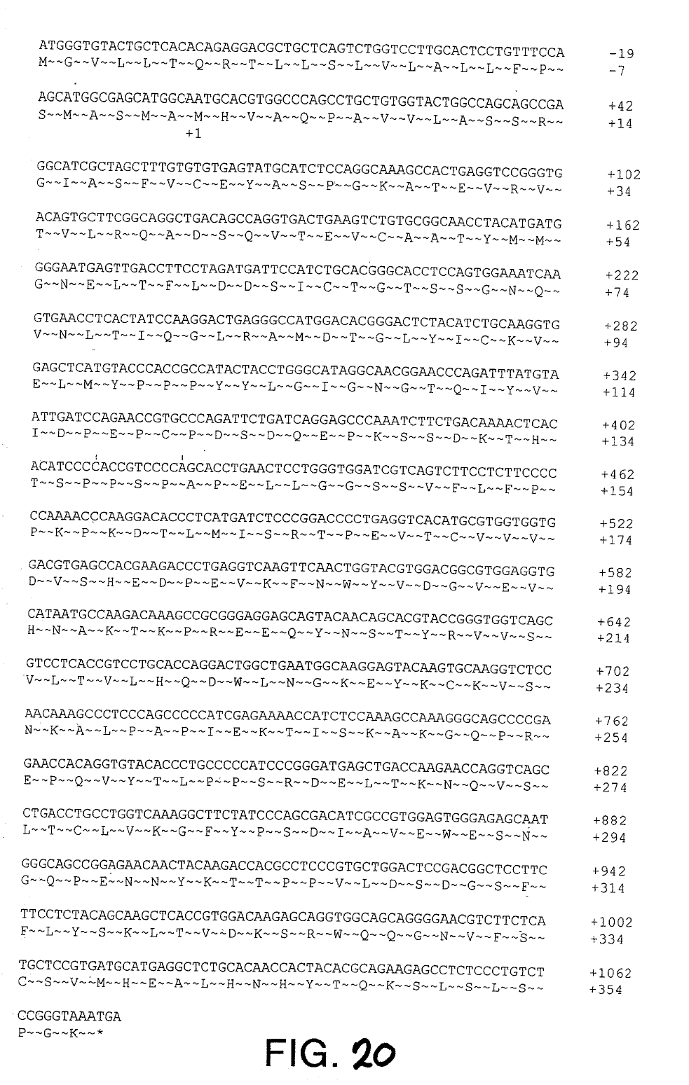

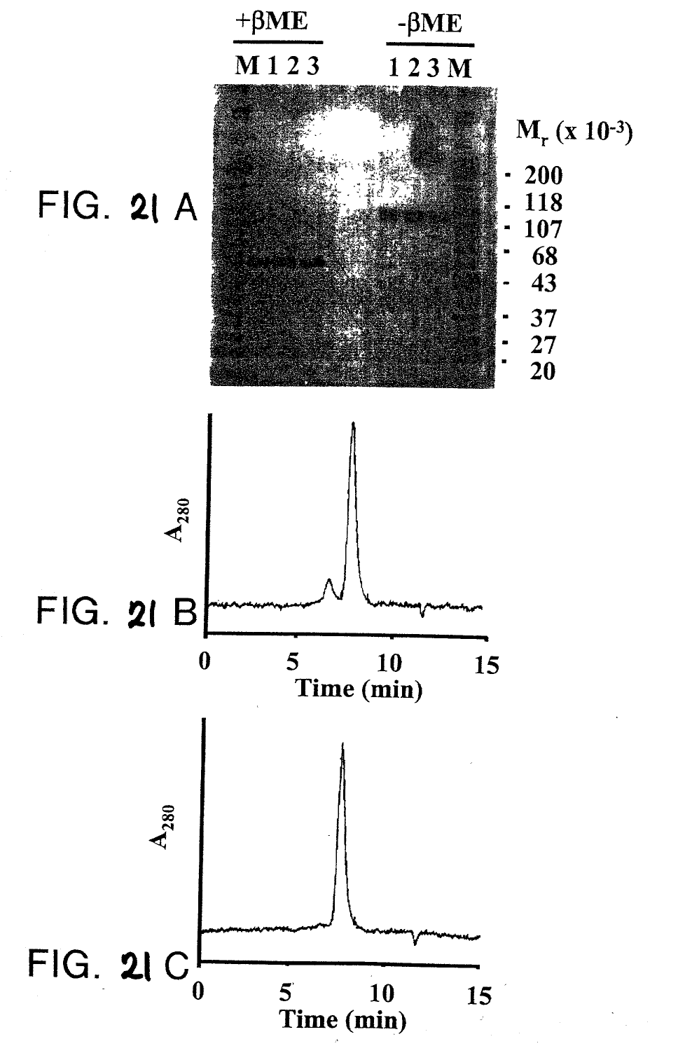

Alternatively, soluble CTLA4 molecules, with mutant or wildtype sequences, may be fusion proteins, wherein the CTLA4 molecules are fused to non-CTLA4 moieties such as immunoglobulin (Ig) molecules that render the CTLA4 molecules soluble. For example, a CTLA4 fusion protein may include the extracellular domain of CTLA4 fused to an immunoglobulin constant domain, resulting in the CTLA4Ig molecule (FIG. 20) (Linsley, P. S., et al., 1994 Immunity 1:793-80).

For clinical protocols, it is preferred that the immunoglobulin region does not elicit a detrimental immune response in a subject. The preferred moiety is the immunoglobulin constant region, including the human or monkey immunoglobulin constant regions. One example of a suitable immunoglobulin region is human Cγ1, including the hinge, CH2 and CH3 regions which can mediate effector functions such as binding to Fc receptors, mediating complement-dependent cytotoxicity (CDC), or mediate antibody-dependent cell-mediated cytotoxicity (ADCC). The immunoglobulin moiety may have one or more mutations therein, (e.g., in the CH2 domain, to reduce effector functions such as CDC or ADCC) where the mutation modulates the binding capability of the immunoglobulin to its ligand, by increasing or decreasing the binding capability of the immunoglobulin to Fc receptors. For example, mutations in the immunoglobulin may include changes in any or all its cysteine residues within the hinge domain, for example, the cysteines at positions +130, +136, and +139 are substituted with serine (FIG. 20). The immunoglobulin molecule may also include the proline at position +148 substituted with a serine, as shown in FIG. 20. Further, the mutations in the immunoglobulin moiety may include having the leucine at position +144 substituted with phenylalanine, leucine at position +145 substituted with glutamic acid, or glycine at position +147 substituted with alanine.

Additional non-CTLA4 moieties for use in the soluble CTLA4 molecules or soluble CTLA4 mutant molecules include, but are not limited to, p97 molecule, env gp120 molecule, E7 molecule, and ova molecule (Dash, B. et al. 1994 J Gen. Virol. 75 (Pt 6):1389-97; Ikeda, T., et al. 1994 Gene 138(1-2):193-6; Falk, K., et al. 1993 Cell. Immunol. 150(2):447-52; Fujisaka, K. et al. 1994 Virology 204(2):789-93). Other molecules are also possible (Gerard, C. et al. 1994 Neuroscience 62(3):721; Byrn, R. et al. 1989 63(10):4370; Smith, D. et al. 1987 Science 238:1704; Lasky, L. 1996 Science 233:209).

The soluble CTLA4 molecule of the invention can include a signal peptide sequence linked to the N-terminal end of the extracellular domain of the CTLA4 portion of the molecule. The signal peptide can be any sequence that will permit secretion of the molecule, including the signal peptide from oncostatin M (Malik, et al., (1989) Molec. Cell. Biol. 9: 2847-2853), or CD5 (Jones, N.H. et al., (1986) Nature 323:346-349), or the signal peptide from any extracellular protein.

The soluble CTLA4 molecule of the invention can include the oncostatin M signal peptide linked at the N-terminal end of the extracellular domain of CTLA4, and the human immunoglobulin molecule (e.g., hinge, CH2 and CH3) linked to the C-terminal end of the extracellular domain (wildtype or mutated) of CTLA4. This molecule includes the oncostatin M signal peptide encompassing an amino acid sequence having methionine at position −26 through alanine at position −1, the CTLA4 portion encompassing an amino acid sequence having methionine at position +1 through aspartic acid at position +124, a junction amino acid residue glutamine at position +125, and the immunoglobulin portion encompassing an amino acid sequence having glutamic acid at position +126 through lysine at position +357.

Specifically, the soluble CTLA4 mutant molecules of the invention, comprising the mutated CTLA4 sequences described infra, are fusion molecules comprising human IgCγ1 moieties fused to the mutated CTLA4 fragments.

In one embodiment, the soluble CTLA4 mutant molecules comprise IgCγ1 fused to a CTLA4 fragment comprising a single-site mutation in the extracellular domain. The extracellular domain of CTLA4 comprises methionine at position +1 through aspartic acid at position +124 (e.g., FIG. 19). The extracellular portion of the CTLA4 can comprise alanine at position −1 through aspartic acid at position +124 (e.g., FIG. 19). Examples of single-site mutations include the following wherein the leucine at position +104 is changed to any other amino acid:

| Single-site mutant: | Codon change: |
|---|---|
| L104EIg | Glutamic acid GAG |
| L104SIg | Serine AGT |
| L104TIg | Threonine ACG |
| L104AIg | Alanine GCG |
| L104WIg | Tryptophan TGG |
| L104QIg | Glutamine CAG |
| L104KIg | Lysine AAG |
| L104RIg | Arginine CGG |
| L104GIg | Glycine GGG |

Further, the invention provides mutant molecules having the extracellular domain of CTLA4 with two mutations, fused to an Ig Cγ1 moiety. Examples include the following wherein the leucine at position +104 is changed to another amino acid (e.g. glutamic acid) and the glycine at position +105, the serine at position +25, the threonine at position +30 or the alanine at position +29 is changed to any other amino acid:

| Double-site mutants: | Codon change: |
|---|---|
| L104EG105FIg | Phenylalanine TTC |
| L104EG105WIg | Tryptophan TGG |
| L104EG105LIg | Leucine CTT |
| L104ES25RIg | Arginine CGG |
| L104ET30GIg | Glycine GGG |
| L104ET30NIg | Asparagine AAT |
| L104EA29YIg | Tyrosine TAT |
| L104EA29LIg | Leucine TTG |
| L104EA29TIg | Threonine ACT |
| L104EA29WIg | Tryptophan TGG |

Further still, the invention provides mutant molecules having the extracellular domain of CTLA4 comprising three mutations, fused to an Ig Cγ1 moiety. Examples include the following wherein the leucine at position +104 is changed to another amino acid (e.g. glutamic acid), the alanine at position +29 is changed to another amino acid (e.g. tyrosine), and the serine at position +25 is changed to another amino acid:

| Triple-site Mutants: | Codon changes: |
|---|---|
| L104EA29YS25KIg | Lysine AAA |
| L104EA29YS25KIg | Lysine AAG |
| L104EA29YS25NIg | Asparagine AAC |
| L104EA29YS25RIg | Arginine CGG |

Soluble CTLA4 mutant molecules may have a junction amino acid residue which is located between the CTLA4 portion and the Ig portion of the molecule. The junction amino acid can be any amino acid, including glutamine. The junction amino acid can be introduced by molecular or chemical synthesis methods known in the art.

The present invention provides CTLA4 mutant molecules including a signal peptide sequence linked to the N-terminal end of the extracellular domain of the CTLA4 portion of the mutant molecule. The signal peptide can be any sequence that will permit secretion of the mutant molecule, including the signal peptide from oncostatin M (Malik, et al., 1989 Molec. Cell. Biol. 9: 2847-2853), or CD5 (Jones, N.H. et al., 1986 Nature 323:346-349), or the signal peptide from any extracellular protein.

The invention provides soluble CTLA4 mutant molecules comprising a single-site mutation in the extracellular domain of CTLA4 such as L104EIg (as included in FIG. 14) or L104SIg, wherein L104EIg and L104SIg are mutated in their CTLA4 sequences so that leucine at position +104 is substituted with glutamic acid or serine, respectively. The single-site mutant molecules further include CTLA4 portions encompassing methionine at position +1 through aspartic acid at position +124, a junction amino acid residue glutamine at position +125, and an immunoglobulin portion encompassing glutamic acid at position +126 through lysine at position +357. The immunoglobulin portion of the mutant molecule may also be mutated so that the cysteines at positions +130, +136, and +139 are substituted with serine, and the proline at position +148 is substituted with serine. Alternatively, the single-site soluble CTLA4 mutant molecule may have a CTLA4 portion encompassing alanine at position −1 through aspartic acid at position +124.

The invention provides soluble CTLA4 mutant molecules comprising a double-site mutation in the extracellular domain of CTLA4, such as L104EA29YIg, L104EA29LIg, L104EA29TIg or L104EA29WIg, wherein leucine at position +104 is substituted with a glutamic acid, and alanine at position +29 is substituted with tyrosine, leucine, threonine or tryptophan, respectively. The sequences for L104EA29YIg, L104EA29LIg, L104EA29TIg and L104EA29WIg, starting at methionine at position +1 and ending with lysine at position +357, plus a signal (leader) peptide sequence are included in the sequences as shown in FIGS. 15-18 respectively. The double-site mutant molecules further comprise CTLA4 portions encompassing methionine at position +1 through aspartic acid at position +124, a junction amino acid residue glutamine at position +125, and an immunoglobulin portion encompassing glutamic acid at position +126 through lysine at position +357. The immunoglobulin portion of the mutant molecule may also be mutated, so that the cysteines at positions +130, +136, and +139 are substituted with serine, and the proline at position +148 is substituted with serine. Alternatively, these mutant molecules can have a CTLA4 portion encompassing alanine at position −1 through aspartic acid at position +124.

The invention provides soluble CTLA4 mutant molecules comprising a double-site mutation in the extracellular domain of CTLA4, such as L104EG105FIg, L104EG105WIg and L104EG105LIg, wherein leucine at position +104 is substituted with glutamic acid and glycine at position +105 is substituted with phenylalanine, tryptophan or leucine, respectively. The double-site mutant molecules further comprise CTLA4 portions encompassing methionine at position +1 through aspartic acid at position +124, a junction amino acid residue glutamine at position +125, and an immunoglobulin portion encompassing glutamic acid at position +126 through lysine at position +357. The immunoglobulin portion of the may also be mutated, so that the cysteines at positions +130, +136, and +139 are substituted with serine, and the proline at position +148 is substituted with serine. Alternatively, these mutant molecules can have a CTLA4 portion encompassing alanine at position −1 through aspartic acid at position +124.

The invention provides L104ES25RIg which is a double-site mutant molecule including a CTLA4 portion encompassing methionine at position +1 through aspartic acid at position +124, a junction amino acid residue glutamine at position +125, and the immunoglobulin portion encompassing glutamic acid at position +126 through lysine at position +357. The portion having the extracellular domain of CTLA4 is mutated so that serine at position +25 is substituted with arginine, and leucine at position +104 is substituted with glutamic acid. Alternatively, L104ES25RIg can have a CTLA4 portion encompassing alanine at position −1 through aspartic acid at position +124.

The invention provides soluble CTLA4 mutant molecules comprising a double-site mutation in the extracellular domain of CTLA4, such as L104ET30GIg and L104ET30NIg, wherein leucine at position +104 is substituted with a glutamic acid, and threonine at position +30 is substituted with glycine or asparagine, respectively. The double-site mutant molecules further comprise CTLA4 portions encompassing methionine at position +1 through aspartic acid at position +124, a junction amino acid residue glutamine at position +125, and an immunoglobulin portion encompassing glutamic acid at position +126 through lysine at position +357. The immunoglobulin portion of the mutant molecule may also be mutated, so that the cysteines at positions +130, +136, and +139 are substituted with serine, and the proline at position +148 is substituted with serine. Alternatively, these mutant molecules can have a CTLA4 portion encompassing alanine at position −1 through aspartic acid at position +124.

The invention provides soluble CTLA4 mutant molecules comprising a triple-site mutation in the extracellular domain of CTLA4, such as L104EA29YS25KIg, L104EA29YS25NIg, L104EA29YS25RIg, wherein leucine at position +104 is substituted with a glutamic acid, alanine at position +29 substituted to tyrosine, and serine at position +25 is changed to lysine, asparagine or arginine, respectively. The triple-site mutant molecules further comprise CTLA4 portions encompassing methionine at position +1 through aspartic acid at position +124, a junction amino acid residue glutamine at position +125, and an immunoglobulin portion encompassing glutamic acid at position +126 through lysine at position +357. The immunoglobulin portion of the mutant molecule may also be mutated, so that the cysteines at positions +130, +136, and +139 are substituted with serine, and the proline at position +148 is substituted with serine. Alternatively, these mutant molecules can have a CTLA4 portion encompassing alanine at position −1 through aspartic acid at position +124.

Additional embodiments of soluble CTLA4 mutant molecules include chimeric CTLA4/CD28 homologue mutant molecules that bind a B7 (Peach, R. J., et al., 1994 J Exp Med 180:2049-2058). Examples of these chimeric CTLA4/CD28 mutant molecules include HS1, HS2, HS3, HS4, HS5, HS6, HS4A, HS4B, HS7, HS8, HS9, HS10, HS11, HS12, HS13 and HS14 (U.S. Pat. No. 5,773,253).

Preferred embodiments of the invention are soluble CTLA4 molecules such as CTLA4Ig (as shown in FIG. 20, starting at methionine at position +1 and ending at lysine at position +357) and soluble CTLA4 mutant L104EA29YIg (as shown in FIG. 15, starting at methionine at position +1 and ending at lysine at position +357). The invention further provides nucleic acid molecules comprising nucleotide sequences encoding the amino acid sequences corresponding to the soluble CTLA4 molecules of the invention. In one embodiment, the nucleic acid molecule is a DNA (e.g., cDNA) or a hybrid thereof. DNA encoding CTLA4Ig (FIG. 20) was deposited on May 31, 1991with the American Type Culture Collection (ATCC), 10801 University Blvd., Manassas, Va. 20110-2209 and has been accorded ATCC accession number ATCC 68629. DNA encoding L104EA29YIg (sequence included in FIG. 15) was deposited on Jun. 19, 2000 with ATCC and has been accorded ATCC accession number PTA-2104. Alternatively, the nucleic acid molecules are RNA or a hybrid thereof.

Additionally, the invention provides a vector, which comprises the nucleotide sequences of the invention. Examples of expression vectors for include, but are not limited to, vectors for mammalian host cells (e.g., BPV-1, pHyg, pRSV, pSV2, pTK2 (Molecular Cloning, A Laboratory Manual, 2 edition, Sambrook, Fritch, and Maniatis 1989, Cold Spring Harbor Press); pIRES (Clontech); pRc/CMV2, pRc/RSV, pSFV1 (Life Technologies); pVPakc Vectors, pCMV vectors, pSG5 vectors (Stratagene)), retroviral vectors (e.g., pFB vectors (Stratagene)), pcDNA-3 (Invitrogen) or modified forms thereof, adenoviral vectors; adeno-associated virus vectors, baculovirus vectors, yeast vectors (e.g., pESC vectors (Stratagene)).

A host vector system is also provided. The host vector system comprises the vector of the invention in a suitable host cell. Examples of suitable host cells include, but are not limited to, prokaryotic and eukaryotic cells. In accordance with the practice of the invention, eukaryotic cells are also suitable host cells. Examples of eukaryotic cells include any animal cell, whether primary or immortalized, yeast (e.g., Saccharomyces cerevisiae, Schizosaccharomyces pombe, and Pichia pastoris), and plant cells. Myeloma, COS and CHO cells are examples of animal cells that may be used as hosts. Particular CHO cells include, but are not limited to, DG44 (Chasin, et al., 1986 Som. Cell. Molec. Genet. 12:555-556; Kolkekar 1997 Biochemistry 36:10901-10909), CHO-K1 (ATCC No. CCL-61), CHO-K1 Tet-On cell line (Clontech), CHO designated ECACC 85050302 (CAMR, Salisbury, Wiltshire, UK), CHO clone 13 (GEIMG, Genova, IT), CHO clone B (GEIMG, Genova, IT), CHO-K1/SF designated ECACC 93061607 (CAMR, Salisbury, Wiltshire, UK), and RR-CHOK1 designated ECACC 92052129 (CAMR, Salisbury, Wiltshire, UK). Exemplary plant cells include tobacco (whole plants, cell culture, or callus), corn, soybean, and rice cells. Corn, soybean, and rice seeds are also acceptable.

The CTLA4 mutant molecules of the invention may be isolated as naturally-occurring polypeptides, or from any source whether natural, synthetic, semi-synthetic or recombinant. Accordingly, the CTLA4 mutant polypeptide molecules may be isolated as naturally-occurring proteins from any species, particularly mammalian, including bovine, ovine, porcine, murine, equine, and preferably human. Alternatively, the CTLA4 mutant polypeptide molecules may be isolated as recombinant polypeptides that are expressed in prokaryote or eukaryote host cells, or isolated as a chemically synthesized polypeptide.

A skilled artisan can readily employ standard isolation methods to obtain isolated CTLA4 mutant molecules. The nature and degree of isolation will depend on the source and the intended use of the isolated molecules.

CTLA4 mutant molecules and fragments or derivatives thereof, can be produced by recombinant methods. Accordingly, an isolated nucleotide sequence encoding wild-type CTLA4 molecules may be manipulated to introduce mutations, resulting in nucleotide sequences that encode the CTLA4 mutant polypeptide molecules. For example, the nucleotide sequences encoding the CTLA4 mutant molecules may be generated by site-directed mutagenesis methods, using primers and PCR amplification. The primers can include specific sequences designed to introduce desired mutations. Alternatively, the primers can be designed to include randomized or semi-randomized sequences to introduce random mutations. Standard recombinant methods (Molecular Cloning; A Laboratory Manual, 2^{nd} edition, Sambrook, Fritch, and Maniatis 1989, Cold Spring Harbor Press) and PCR technology (U.S. Pat. No. 4,603,102) can be employed for generating and isolating CTLA4 mutant polynucleotides encoding CTLA4 mutant polypeptides.

The invention includes pharmaceutical compositions for use in the treatment of immune system diseases comprising pharmaceutically effective amounts of soluble CTLA4 molecules. In certain embodiments, the immune system diseases are mediated by CD28/CTLA4/B7 interactions. The soluble CTLA4 molecules are preferably soluble CTLA4 molecules with wildtype sequence and/or soluble CTLA4 molecules having one or more mutations in the extracellular domain of CTLA4. The pharmaceutical composition can include soluble CTLA4 protein molecules and/or nucleic acid molecules, and/or vectors encoding the molecules. In preferred embodiments, the soluble CTLA4 molecules have the amino acid sequence of the extracellular domain of CTLA4 as shown in either FIG. 20 or 15 (CTLA4Ig or L104EA29Y, respectively). Even more preferably, the soluble CTLA4 mutant molecule is L104EA29YIg as disclosed herein. The compositions may additionally include other therapeutic agents, including, but not limited to, drug toxins, enzymes, antibodies, or conjugates.

As is standard practice in the art, pharmaceutical compositions, comprising the molecules of the invention admixed with an acceptable carrier or adjuvant which is known to those of skill of the art, are provided. The pharmaceutical compositions preferably include suitable carriers and adjuvants which include any material which when combined with the molecule of the invention (e.g., a soluble CTLA4 molecule, such as, CTLA4Ig or L104EA29Y) retains the molecule's activity and is non-reactive with the subject's immune system. These carriers and adjuvants include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, phosphate buffered saline solution, water, emulsions (e.g. oil/water emulsion), salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances and polyethylene glycol. Other carriers may also include sterile solutions; tablets, including coated tablets and capsules. Typically such carriers contain excipients such as starch, milk, sugar (e.g. sucrose, glucose, maltose), certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods. Such compositions may also be formulated within various lipid compositions, such as, for example, liposomes as well as in various polymeric compositions, such as polymer microspheres.

Kits comprising pharmaceutical compositions therapeutic for immune system disease are also encompassed by the invention. In one embodiment, a kit comprising one or more of the pharmaceutical compositions of the invention is used to treat an immune system disease. For example, the pharmaceutical composition comprises an effective amount of soluble CTLA4 mutant molecules that bind to B7 molecules on B7-positive cells, thereby blocking the B7 molecules from binding CTLA4 and/or CD28 on T-cells. Further, the kit may contain one or more immunosuppressive agents used in conjunction with the pharmaceutical compositions of the invention. Potential immunosuppressive agents include, but are not limited to, corticosteroids, nonsteroidal antiinflammatory drugs (e.g. Cox-2 inhibitors), cyclosporin prednisone, azathioprine, methotrexate, TNFα blockers or antagonists, infliximab, any biological agent targeting an inflammatory cytokine, hydroxychloroquine, sulphasalazopryine, gold salts, etanercept, and anakinra.

The following examples are presented to illustrate the effects of using busulfan and T cell-depleted bone marrow to establish chimerism in subjects. The examples also illustrate the effects of using busulfan, T cell-depleted bone marrow, and costimulatory blockade to inhibit organ tissue transplant rejections and to treat hemoglobinopathies. The methodology and results may vary depending on the intended goal of treatment and the procedures employed. The examples are not intended in any way to otherwise limit the scope of the invention.

### EXAMPLES

General Methods

Mice.

Adult male 6-8 week old C57BL/6 (H-2^{b}), Balb/c (H-2^{d}), C3H/HeJ (H-2^{k}), C57BL/6Scid (H-2^{b}) mice were obtained from Jackson Laboratories (Bar Harbor, ME). C57BL/6JHbb^{d3th} male mice (H-2^{b}) were provided by Dr. David Archer. All mice were housed in specific pathogen free conditions and in accordance with institutional guidelines.

Bone marrow preparation and treatment regimens. Bone marrow was flushed from tibiae, femurs and humeri using conventional techniques. Ferro-magnetic T cell depletion with anti-CD3 (Pharmingen, San Diego, Calif.) or anti-CD90 antibodies, and magnetic cell sorting (MACs) separation column system (Miltenyi, Auburn, Calif.) was performed and confirmed by flow cytometry (anti-CD3, anti-CD4, anti-CD8 and anti-CD5 antibodies, Pharmingen, San Diego, Calif.). Red cell lysis was performed using a Trizma base ammonium chloride solution. The bone marrow cells were resuspended at 2×10⁷ cells/500 μl sterile saline and injected intravenously into the recipient subjects. In certain experiments, hamster anti-mouse CD40L (MR1, Bioexpress, Lebanon, N.H.) and CTLA4-Ig (Bristol-Myers Squibb, Princeton, N.J.) were administered on days 0, 2, 4, 6, 14, and 28 (500 μg/dose i.p.); day 0 representing the day of the transplant of the bone marrow. In-vivo depletion of CD4+ T cells was accomplished by administering 100 μg anti-CD4 mAb (GK1.5) intraperitoneally on days −3, −2, −1, 0, and weekly thereafter; day 0 representing the day of the transplant.

Skin Grafting.

Full thickness skin grafts (˜1 cm²) were transplanted on the dorsal thorax of recipient mice and were secured with a Band-Aid® for 7 days.

Flow Cytometric Analysis.

Peripheral blood was analyzed by staining with fluorochrome-conjugated antibodies (anti-CD3, anti-CD5, anti-CD11b, anti-GR1, anti-B220, anti-H-2K^{d}, anti-H-2 K^{b}, anti-Vβ11, anti-Vβ5.1/5.2, anti-Vβ8.1/8.2 (Pharmingen), anti-CD4, anti-CD8 (Caltag Laboratories, Burlingame, Calif.), or immunoglobulin isotype controls (Pharmingen)), followed by red blood cell lysis and washing with a whole blood lysis kit (R+D Systems, Minneapolis, Minn.). Stained cells were analyzed using Cellquest software on a FACSCalibur flow cytometer (Becton Dickinson, Mountain View, Calif.).

Cytotoxicity Assays.

Balb/c CL.7 cells were used as targets and were suspended at 1×10⁷/ml with 750 μCi ⁵¹Cr (NEN Life Science Products, Boston, Mass.) for 90 minutes at 37° C. Target cells were washed three times and plated at 1×10⁴ targets/well. Effectors were prepared as nylon wool passaged splenocytes and plated at the appropriate ratios in quadruplicate. Total lysis was measured by addition of 2% Triton-X to targets, and spontaneous lysis by the addition of R-10 without effector cells. After 5 hours, the supernatant was harvested and analyzed by γ-counting. Percent specific lysis was determined by use of the following formula: 100×(cpm unknown-cpm spontaneous)/(cpm total-cpm spontaneous).

IFNγ ELISpot Assays.

Allospecific T-cell responses were measured by an IFNγ Enzyme-Linked Immunospot (ELISpot) assay using nylon wool passed splenocytes from experimental C57BL/6 mice. The capture antibody, rat anti-mouse IFNγ (clone R4-6A2; Pharmingen), was incubated at 4 μg/ml in phosphate-buffered saline (PBS) (100 μl/well) at 4° C. overnight in ester-cellulose-bottom plates (Millipore, France). After washing, various dilutions of effector cells were added. Stimulators, donor dendritic cells obtained by overnight transient adherence, were irradiated (2000 rads) and added at a 1:10 stimulator to effector ratio. Effector cells were incubated for 14-16 hours at 37° C. with or without stimulators. After the culture period, biotinylated anti-mouse IFNγ (clone XMG1.2; Pharmingen) was added at 4 μg/ml (100 μl per well). After 2-3 hours at 4° C., unbound antibody was removed, and horseradish peroxidase-avidin D (Sigma, St. Louis, Mo.) was added. Spots were developed with the substrate 3-amino-9-ethyl-carbazole (Sigma) with 0.015% H₂O₂. Each spot represents an IFNγ-secreting cell, and the frequency was determined by dividing the number of spots counted in each well by the total number of cells plated at that dilution.

CFSE Assay.

Splenic and mesenteric lymph node cells were harvested from experimental mice. After red blood cell lysis and nylon wool passage, cells were incubated in 10 μM carboxyfluorescein succinimidyl ester (CFSE; Molecular Probes, Eugene, Oreg.). Irradiated (1800 rads) Balb/c, C57BL/6, or C3H mice then intravenously received 1×10⁷-1×10⁹ CFSE labeled cells. After 66-72 hours, splenocytes were harvested from the recipients, the red blood cells lysed, and the remaining cells stained with anti-CD4 and anti-CD8 (Pharmingen) and analyzed by flow cytometry as described above. The concentration of CFSE within the cell decreases by 50% after each division.

Hematologic Monitoring.

Hemavet™ series multiple species hematology instrument (1500 R series, CDC technologies, Oxford, Conn.) was used to determine the complete blood counts.

Hemoglobin Electrophoresis.

Hemoglobin electrophoresis was performed using a cystamine hemoglobin cellulose acetate gel electrophoresis procedure (Whitney et al., Biochem. Genet., 16:667-672 (1978)). Briefly, 2 μl of whole blood was mixed with 7 μl of a solution containing 83 mM cystamine, 0.25% ammonium hydroxide and 0.01M dithiothreitol (DTT). The mixture was incubated at room temperature for 15 minutes before applying to cellulose acetate gels (Helena Labs, Beaumont, Tex.) and electrophoresed for 45 minutes at 350 volts in SupraHeme buffer (Helena Labs). Gels were post-stained using Ponceau S (Sigma, St. Louis, Mo.) for hemoglobin visualization.

Reticulocyte Counts.

Reticulocytes were quantified by staining whole blood with the RNA-specific label Thiazole Orange (Sigma, St. Louis, Mo.), anti-CD45, and Ter-119 antibodies (Pharmingen, San Diego, Calif.). Reticulocytes are defined as cells that are Ter-119 positive, Thiazole Orange-positive, and CD45-negative.

### Example 1

Blockade of Costimulatory Pathways and Administration of Busulfan Permits Titratable Mixed Chimerism Without Myelosuppression.

This example demonstrates that administration of busulfan to a subject permits titratable mixed chimerism without myelosuppression.

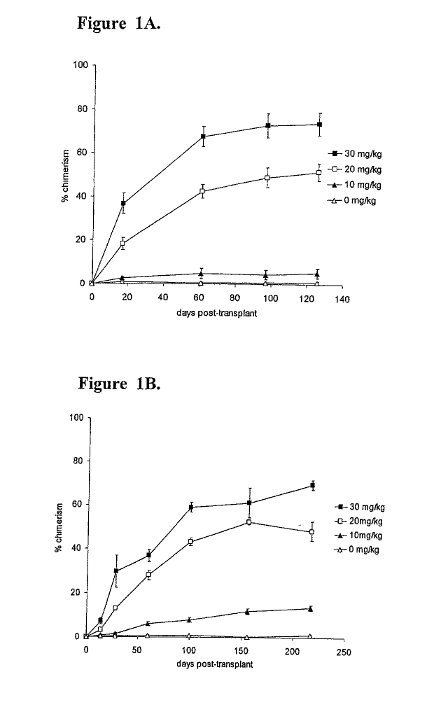

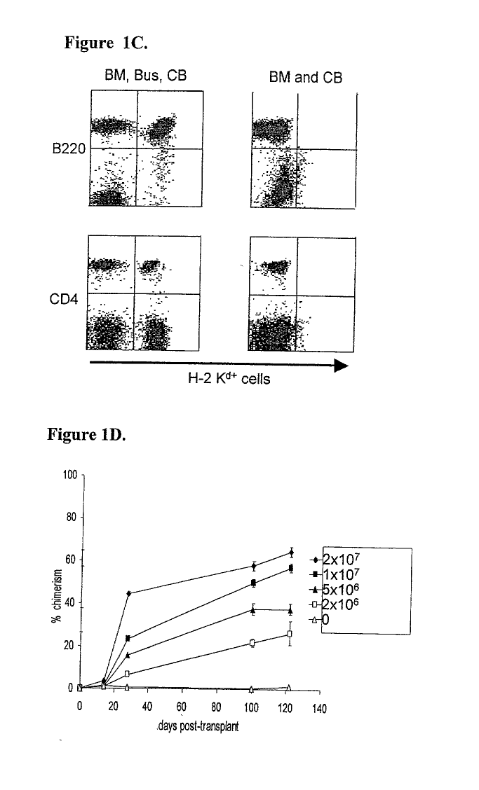

C57BL/6 (B6) recipient mice (H₂b, CD45.2) were administered a single busulfan dose (0 mg/kg, 10 mg/kg, 20 mg/kg, or 30 mg/kg, i.p.; below the LD50 dose of 136 mg/kg, with marrow rescue (Yeager et al., supra.)) one day before intravenous infusion of 2×10⁷ B6.5JL (H-2^{b}, CD45.1) T cell-depleted bone marrow cells. Levels of donor hematopoietic chimerism, measured by peripheral blood cell flow cytometry, as described above, were directly proportional to the administered busulfan dose (FIG. 1A). Similar results were achieved when the busulfan was administered six or twelve hours before the administration of the T cell-depleted bone marrow cells.

The ability of a similar “micro-conditioning” regimen to induce mixed allogeneic chimerism and transplant tolerance in the context of costimulation blockade was examined. Administration of a “tolerizing” dose of donor bone marrow cells together with blockade of costimulatory pathways (e.g., CD28/B7 and CD40/CD40L; costimulation blockade) should inactivate donor-reactive peripheral T cells (Sayegh et al., Transplantation, 64:1646-1650 (1997); Pearson et al., Transplantation, 61:997-1004 (1996); Markees et al., J. Clin. Invest., 101:2446-2455 (1998)).

Five days after the initial donor cell infusion, a single dose of busulfan was administered, followed the next day by a second “engrafting” dose of allogeneic T cell-depleted bone marrow. In particular, B6 mice were intravenously administered allogeneic T cell-depleted bone marrow (Balb/c (H-2^{d}) 2×10⁷ cells; day 0 and day 6), costimulation blockade (500 μg CTLA4-Ig and anti-CD40L; day 0, day 2, day 4, day 6, day 14, and day 28), and varying doses of busulfan (0 mg/kg, 10 mg/kg, 20 mg/kg, and 30 mg/kg; day 5). Control groups included animals that received either no treatment, T cell-depleted bone marrow alone, busulfan and T cell-depleted bone marrow, busulfan alone, costimulation blockade alone, or T cell-depleted bone marrow and costimulation blockade.

All animals receiving the experimental treatment developed high-level, multi-lineage hematopoietic chimerism persisting for >220 days (FIG. 1B). As in the congenic experiment, the level of chimerism was directly proportional to the dose of busulfan. Animals in control groups failed to demonstrate hematopoietic chimerism at any time point (FIGS. 1B and 1C). The levels of hematopoietic chimerism seen in allogeneic transplants were similar to the levels seen in mice receiving congenic T cell-depleted bone marrow, indicating that the addition of donor cells and costimulation blockade had effectively eliminated the immunological barrier to allogeneic T cell-depleted bone marrow transplantation.

While initial experiments achieved high-level chimerism using an engrafting dose of T cell-depleted bone marrow that was only one tenth the quantity used in recent reports without recipient conditioning (Wekerle et al., Nature Medicine, 6:464-469 (2000); Durham et al., Journal of Immunology, 165:1-4 (2000)), it was conceivable that lower doses of T cell-depleted bone marrow could induce stable mixed chimerism.

The engrafting dose of T cell-depleted bone marrow (day 6) was titrated from 2×10⁷ to 0. On day >120 post-transplant, peripheral donor cells correlated directly with the engrafting dose of T cell-depleted bone marrow (2×10⁷-65%, 1×10⁷-57%, 5×10⁶-37%, 2×10⁶-26%; FIG. 1D). Stable macrochimerism was achieved by using an engrafting dose of as few as 2×10⁶ T cell-depleted bone marrow cells (10-fold lower T-cell depleted bone marrow cells than previous reports using non-myelosuppressive regimens).

These results indicate that the level of chimerism attained is titratable either by altering the dose of bone marrow or by modifying the dose of busulfan. In other experiments either irradiated bone marrow or splenocytes could be substituted for the “tolerizing” dose of bone marrow.

Tomita et al. reported that 3Gy whole body irradiation (WBI) was the minimal dose required to produce reliable long-term engraftment of syngeneic pluripotent hematopoietic stem cells. In addition, they evaluated the toxicity profile associated with WBI-based bone marrow transplant protocols and concluded that 3Gy was essentially non-myelosuppressive (Tomita et al., Blood, 83:939-948 (1994)). Furthermore a similar protocol involving 3Gy WBI and costimulatory blockade has been proven sufficient to produce reliable levels of chimerism in an allogeneic model (Wekerle et al., J. Exp. Med., 187:2037-2044 (1998)).

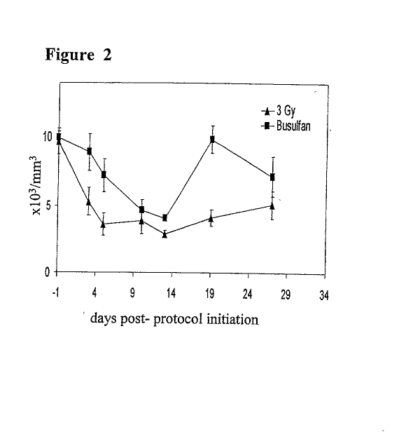

For comparison, the toxicity of the busulfan based protocol (2×10⁷ Balb/c T cell-depleted bone marrow to C57BL/6 recipient with 500 μg costimulation blockade given at days 0, 2, 4, and 6; 20 mg/kg busulfan day −1; n=5) and an irradiation based protocol (2×10⁷ Balb/c donor bone marrow cells into C57BL/6 recipient with 450 μg anti-CD40L day 0 and 500 μg CTLA4-Ig day 2, 3Gy irradiation day 0; n=5) were assessed. As shown in FIG. 2, both protocols are essentially non-myelosuppressive (white cell count nadir: irradiation-based protocol-day 13, 2.86×10³/mm³, busulfan-based protocol-day 13, 4.04×10³/mm³). Furthermore, greater than 200 animals have been treated with the busulfan based regimen with only 1 death (resulting from anesthesia). These results demonstrate that titratable, high level chimerism can be achieved in the absence of gamma irradiation.

### Example 2

Costimulation Blockade/Busulfan Regimen Corrects Hemoglobinopathies.

This example demonstrates the effects of the micro-conditioning, costimulation blockade chimerism induction protocol in experimental hemoglobinopathy models.

The degree to which the chimerism induction protocol could promote replacement of the red cell compartment in the Hbb^{th2} murine model of β-thalassemia was assessed (Shehee et al. Proc. Natl. Acad. Sci. USA, 90:3177-3181 (1993)). This β-thalassemia model, created by insertional disruption of the mouse adult β-major globin gene, results in perinatal death of homozygotes, whereas heterozygotes survive but display a phenotype similar to human β-thalassemia intermedia, characterized by shortened red blood cell survival, anemia, and reticulocytosis.

β-thalassemic heterozygote recipients (H-2^{b}) were treated with a tolerizing dose (2×10⁷ cells) of Balb/c T cell-depleted bone marrow (day 0), costimulation blockade (days 0, 2, 4, 6), 20 mg/kg of busulfan (day 5) and an engrafting dose (2×10⁷ cells) of Balb/c T cell-depleted bone marrow (day 6). Control recipients received costimulation blockade and T cell-depleted bone marrow without busulfan. Assessments of leukocyte and red cell chimerism, hemoglobin levels (Hb) and reticulocyte counts were performed prior to protocol induction, and at 2 weeks, 4 weeks, and monthly following bone marrow transplantation.

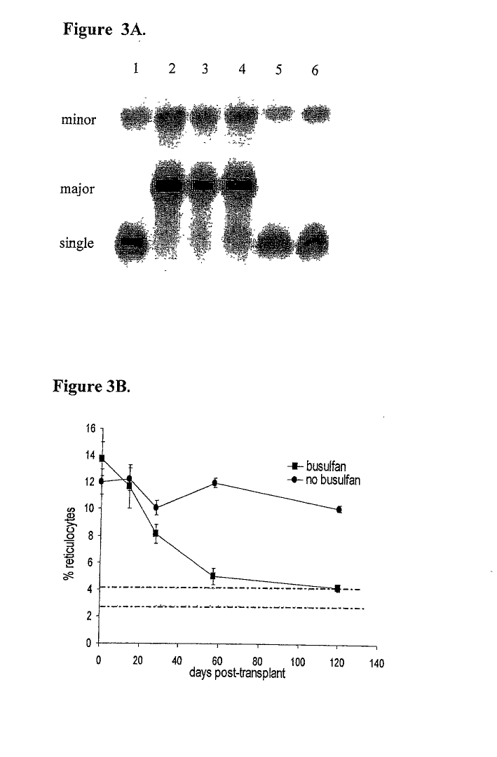

As in the previous experiments using B6 recipients, leukocyte chimerism developed in recipients treated with costimulation blockade, T cell-depleted bone marrow and busulfan, but not in recipients receiving only costimulation blockade and T cell-depleted bone marrow. Furthermore, near complete replacement of the pathologic Hbβ band by the functional Balb/c major Hbβ allele was observed in the chimeric recipients, but not in the control group (FIG. 3A). Lane 1 demonstrates untreated Thalassemic Hb (minor and single bands) and lane 2 demonstrates donor (Balb/c, minor and major bands). Lanes 3-4 represent thalassemic animals (>day 150) that received busulfan on day 5 (20 mg/kg), allogeneic T cell-depleted bone marrow (Balb/c) (days 0 and 6) and costimulation blockade. The abnormal thalassemic Hb is almost completely replaced by normal Balb/c hemoglobin. Lanes 5-6 show Hb from thalassemic animals that were treated with bone marrow and costimulation blockade, but without busulfan. It is clearly evident that the only Hb present is recipient derived.

Prior to protocol induction, percent reticulocytes in thalassemic peripheral blood was 12.0% in animals not receiving busulfan (FIG. 3B; closed circles, n=3) and 13.7% (FIG. 3B; closed squares, n=3) in animals treated with busulfan. By day 120 after protocol induction, those animals treated with busulfan had normalized their reticulocytosis (4.2%) while non-chimeric animals maintained abnormally high levels of reticulocytes in their peripheral blood (10.1%). The grey bar represents normal reticulocyte counts in wild type B6 animals (n=10). Error bars represent standard error of the mean. Reticulocyte counts and hemoglobin levels (Hb) in the chimeric, thalassemic mice normalized, indicating that the pathogenesis of the disorder had been eliminated.

### Example 3

Costimulation Blockade/Busulfan Protocol Promotes Organ Tissue Transplant Tolerance.

This example demonstrates the effects of the micro-conditioning, costimulation blockade chimerism induction protocol in solid organ tissue transplants. To test whether the protocol of “micro-conditioning” and costimulation blockade could induce tolerance to solid organ allografts placed at the outset of the protocol, an immunologically rigorous (Balb/c to B6) skin graft model was employed.

B6 mice received 2×10⁷ Balb/c T cell-depleted bone marrow cells, costimulation blockade, and busulfan (20 mg/kg), as described above. In addition, animals received a day 0 Balb/c skin graft.

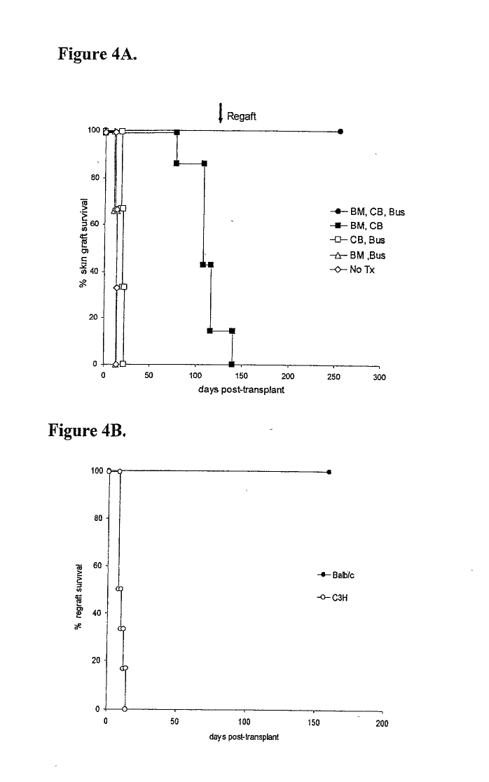

Control groups (no treatment, open diamonds, n=3; T cell-depleted bone marrow and busulfan, open triangles, n=3; or costimulation blockade and busulfan; open squares, n=3) all promptly rejected Balb/c allografts (FIG. 4A). Recipients receiving T cell-depleted bone marrow and costimulation blockade without busulfan (closed squares, n=7) showed greatly prolonged survival (FIG. 4A), but ultimately rejected their allografts.

In contrast, animals receiving busulfan, T cell-depleted bone marrow and costimulation blockade (closed circles, n=7) accepted their skin grafts for >250 days without evidence of rejection (FIG. 4A). Similar results were obtained in the reciprocal strain combination. Importantly, from a clinical perspective, the concomitant placement of a donor-specific skin graft did not prevent the development of hematopoietic chimerism.

One hundred days after protocol initiation animals were re-challenged with a second donor (Balb/c, H-2^{d}) and third party (C3H, H-2^{k}) skin grafts. At 100 days, primary skin graft survival in bone marrow, costimulation blockade group (closed squares, n=7) was 86% while animals receiving costimulation blockade, bone marrow, busulfan (closed circles, n=7) enjoyed 100% acceptance. Following placement of second donor skin graft, animals receiving bone marrow and costimulation blockade without busulfan quickly rejected both the primary and secondary donor skin grafts (median survival time (MST) 7 days). By contrast, primary skin grafts placed on animals treated with costimulation blockade, bone marrow, busulfan survived indefinitely (>250 days) even following regrafting with a second donor specific skin graft.

Next, the animals that received bone marrow, costimulation blockade, and busulfan were re-challenged approximately 100 days after the original transplant with donor (Balb/c) or third-party (C3H/HeJ) skin grafts (FIG. 4B). Control animals promptly rejected both Balb/c and C3H/HeJ skin grafts (MST 10 days and 12 days, respectively). Chimeric animals quickly rejected the third party skin graft (FIG. 4B, open circles, MST 10 days) while the secondary donor grafts went on to 100% survival for over 150 days (FIG. 4B, closed circles). Similar results have been achieved in additional experiments.

Administration of T cell-depleted bone marrow and blockade of costimulatory pathways without the induction of mixed chimerism (i.e. the group receiving costimulation blockade and T cell-depleted bone marrow but no busulfan) significantly prolonged primary allograft survival but did not promote lasting tolerance (original graft MST 107 days, donor specific re-graft MST 8 days). In contrast, mice that received busulfan, T cell-depleted bone marrow cells, and costimulation blockade became high-level chimeras, uniformly accepted the second donor-specific Balb/c skin grafts (MST>125 days), and promptly rejected C3H/HeJ grafts (MST 10 days, FIG. 4B). Importantly, the original Balb/c skin grafts and the chimeric state were unperturbed following re-challenge (FIG. 4A).

In addition, robust tolerance and stable chimerism using a single dose of 2×10⁷ T cell-depleted bone marrow cells on the day of skin transplantation with a single dose of busulfan -24, -12, or -6 hours before T cell-depleted bone marrow was achieved.

### Example 4

Donor Bone Marrow and Costimulation Blockade Transiently Eliminates Anti-Donor T Cell Responses but Mixed Chimerism is Required for Permanent Tolerance.

The ability of the tolerant and non-tolerant mice to generate anti-donor T cell cytolytic (CTL) and IFNγ (ELISpot) responses after challenge with a donor skin graft both at early (day 10) and late (>day 100) time points was examined. Splenic T cells were prepared from B6 recipients of Balb/c skin grafts that received either T cell-depleted bone marrow and costimulation blockade, T cell-depleted bone marrow and busulfan, T cell-depleted bone marrow and costimulation blockade with busulfan, no treatment, or from naïve B6 animals.

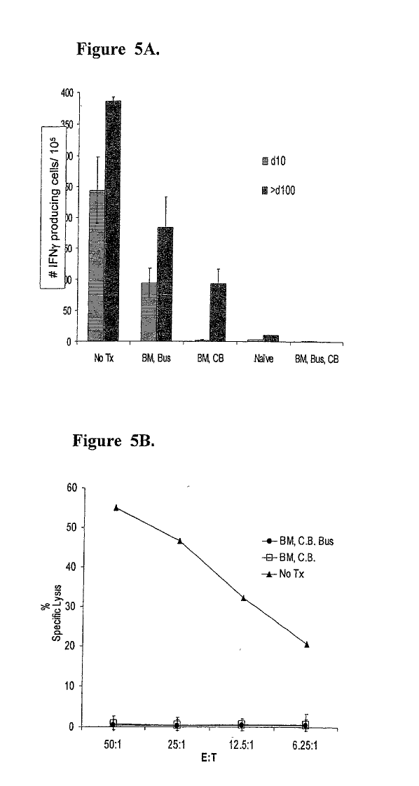

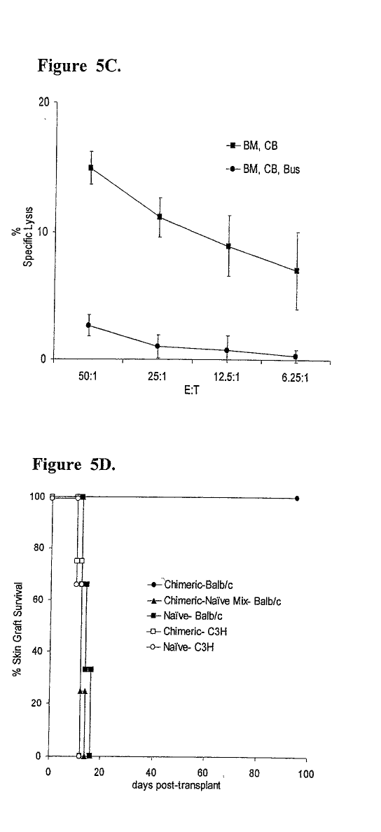

Untreated B6 mice generated both large numbers of IFNγ producing cells (FIG. 5A) and strong CTL responses (FIG. 5B) 10 days after skin grafting. During the induction period (at day 10) both the generation of IFNγ producing cells and CTL responses were inhibited in all groups receiving costimulation blockade and essentially abrogated in animals receiving costimulation blockade and bone marrow (with or without busulfan, FIGS. 5A and 5B).

However at later time points (100 days after initial skin grafting and induction of tolerance protocol), animals treated with T cell-depleted bone marrow and costimulation blockade without busulfan generated significant numbers of donor-reactive IFNγ producing cells and anti-donor CTL activity after re-challenge with a second donor skin graft; in contrast, those treated with T cell-depleted bone marrow, costimulation blockade and busulfan failed to mount any anti-donor CTL activity or IFNγ response (FIGS. 5A and 5C). Both groups mounted similar anti-third party (C3H, H-2^{k}) responses. Similar results have been observed in two additional experiments.

These results indicate that the initial, transient hypo-responsiveness to donor antigen established by T cell-depleted bone marrow in the presence of costimulation blockade wanes over time, possibly due to the emergence of new thymic emigrants or to the decay of regulatory T cell function. In contrast, the addition of a single, non-myelosuppressive dose of busulfan, prior to the engrafting dose of bone marrow, permitted sufficient donor hematopoietic chimerism to result in robust, long-lasting donor specific tolerance.

### Example 5

Recipient CD4⁺ T Cells are Required for the Development of Chimerism and Tolerance but not for Maintenance.

Previous reports have indicated that long-term survival induced by CD40/CD40L blockade and donor-specific transfusion requires the participation of CD4⁺ T cells (Markees et al., J. Clin. Invest., 101:2446-2455 (1998)). However, it not is known whether protocols that induce tolerance via the establishment of mixed chimerism also require CD4⁺ T cells.

To explore this question, bone marrow recipients were depleted of CD4⁺ T cells in vivo with an anti-CD4 monoclonal antibody, prior to and during tolerance induction. In the absence of CD4+cells, animals treated with donor bone marrow, 20 mg/kg busulfan, and costimulation blockade (as above) uniformly failed to become chimeric, implying an essential role for CD4+cells during chimerism induction (skin graft MST 29 days).

To investigate whether CD4⁺ cells were also necessary for tolerance/chimerism maintenance we depleted long-term chimeras (>300 days) of CD4⁺ cells as above. In contrast to the induction phase, where CD4⁺ cells play a pivotal role, during the maintenance phase, depletion of CD4⁺ cells did not perturb either skin graft survival or the chimeric state.

Because there is strong evidence that dominant regulatory mechanisms may play a crucial role in tolerance maintenance in other costimulation blockade models, we also performed adoptive transfer experiments to test for evidence of regulation (Honey et al., J. Immunol., 163:4805-4810 (1999)). We adoptively transferred T cells from tolerant-chimeric mice, T cells from naïve B6 mice, or mixtures of tolerant and naive T cells, into C57BL/6 Scid mice (B6 Scid) receiving both Balb/c and C3H skin grafts. At approximately 150 days after therapy institution (last T cell-depleted bone marrow on day 6 and last costimulation blockade on day 28), T cells were prepared from the spleens of mice that had been rendered specifically tolerant to Balb/c skin grafts (but rejected third party) with our protocol. Next, B6 Scid mice received 5×10⁶ transferred T cells from chimeric-tolerant animals (T cell-depleted bone marrow, costimulation blockade, busulfan), cells from tolerant animals mixed with 5×10⁶ T cells from naïve B6 mice or only cells from naïve B6 mice.

T cells from naive animals quickly rejected donor (FIG. 5D, closed squares) and third party grafts (MST 10 and 12 days respectively, FIG. 5D, open circles). In contrast, 100% of animals receiving T cells from tolerant animals (closed circles) accepted Balb/c skin grafts (>75 days) while rejecting third party allografts (MST=12 days, FIG. 5D). When naïve T cells were mixed with the tolerant T cells however, prompt rejection of the Balb/c skin grafts was observed (MST =12 days, FIG. 5D, closed triangles).

These data confirm that T cells from animals receiving our protocol of T cell-depleted bone marrow, busulfan and costimulation blockade are robustly and specifically tolerant to the marrow donor and suggest that while regulatory mechanisms may play an important role during tolerance induction, they are unlikely to be the major mechanism by which tolerance is maintained in this model.

### Example 6

Clonal Deletion of Alloreactive T Cells is the Main Mechanism for Tolerance Maintenance.

To determine whether the tolerant state was associated with clonal deletion of donor reactive T cells, utilization of Vβ TCR segments before, during, and after tolerance induction was examined.

Balb/c mice delete Vβ11 and Vβ5 bearing T cells whereas B6 mice do not express the class II MHC molecule, I-E, and utilize Vβ11 on ˜4-5% of CD4⁺ T cells and Vβ5.1/5.2 on ˜2-3% of CD4⁺ T cells (Dyson et al., Nature, 349:531-534 (1991); Bill et al., J. Exp. Med., 169:1405-1419 (1989).

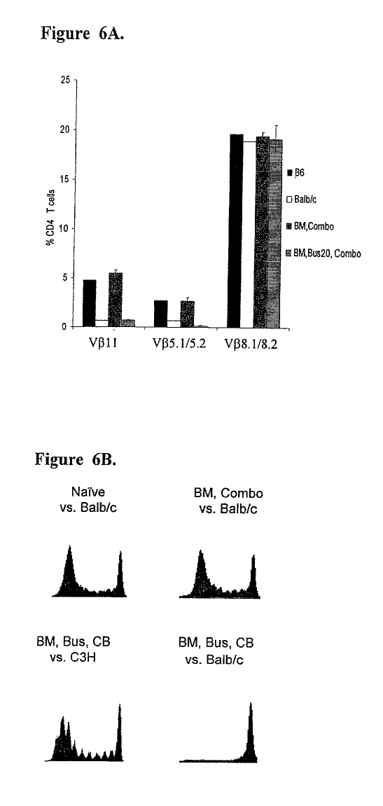

Control groups (costimulation blockade or T cell-depleted bone marrow or busulfan alone) failed to delete donor reactive Vβ11⁺ or Vβ5⁺CD4⁺ T cells (FIG. 6A). The Vβ11⁺ and Vβ5.1/5.2⁺ levels were consistent with wild type B6 levels (4-5% and 2-3%, respectively). In contrast, recipients of Balb/c T cell-depleted bone marrow, busulfan, and costimulation blockade therapy developed near complete deletion of CD4⁺Vβ11⁺ and CD4⁺Vβ5⁺ T cells by day 60. The percentage of Vβ8 bearing CD4⁺ T cells, which are expressed on approximately 15-20% of Balb/c and B6 CD4⁺ T cells, was similar in all groups, indicating that the T cell deletion was donor specific in nature (FIG. 6A). Similar results have been observed in >100 mice from multiple experiments.

As the MMTv system serves as a surrogate marker for alloreactivity, an in vivo allo-proliferation graft versus host disease (GvHD) assay was performed to directly test for the presence of residual alloreactivity (Lyons et al., Journal of Immunological Methods, 171:131-137 (1994)). T cells from chimeric (T cell-depleted bone marrow, costimulation blockade, busulfan), non-chimeric (T cell-depleted bone marrow, costimulation blockade), and naïve animals were harvested from spleens and mesenteric lymph node (LN) (T cells harvested from experimental animals >100 days after transplant). After labeling with 10 μM CFSE, T cells were transferred into recipient mice (Balb/c or C3H), previously supra-lethally irradiated with 1800 rads. After 72 hours, splenocytes were harvested and analyzed via flow cytometry.

While CD4⁺ and CD8⁺T cells from both the naive and non-chimeric groups underwent extensive cell division in response to Balb/c hosts, T cells from the tolerant mice generated no anti-donor proliferative response (FIG. 6B). Strong proliferative responses to third party (C3H), however, were similar in all groups (FIG. 6B). Taken together with repertoire analysis, the absence of CD4⁺ or CD8⁺T cells capable of cellular division in this graft versus host disease model provides further evidence that the tolerant state achieved with this protocol results in near complete elimination of the donor-specific T cells. Comparable results have been observed in additional experiments.

### Example 7

Non-Myeloablative Allogeneic Bone Marrow Transplantation Treats Sickle Cell Disease

A transgenic knockout mouse that lacks all murine hemoglobins and instead produces exclusively human α, γ, and sickle-β-globin (Paszty et al., Science, 278:876-878 (1997)) was used to test the ability of the transplant regimens described herein to treat sickle cell disease. This mouse model replicates much of the complex multi-organ disease characteristics present in human sickle cell disease patients.

The results disclosed herein demonstrate for the first time that non-myeloablative preconditioning with busulfan coupled with costimulation blockade can produce a consistent phenotypic cure of murine sickle cell disease through stable mixed chimerism. Furthermore, this cure is accomplished with fully MHC-mismatched donor marrow.

Methods

Sickle mice were supplied by Dr. Paszty at the Lawrence Berkley National Laboratory and are currently maintained at Emory University. Transplant recipients (males; 7-12 weeks) expressing exclusively human α and β^{Sickle} globin were bred by selective mating, and exist on a mixed genetic background (strains: FVB/N; 129; DBA/2; C57BL/6; and Black Swiss). BALB/c mice were used as bone marrow donors. BALB/c and C3H/HeJ mice were used for tests of donor-specific tolerance, and C57BL/6 mice were used as hematologically normal control mice.

Recipient mice received 2×10⁷ BALB/c, T-cell depleted (with anti-CD3, anti-CD4, anti-CD8 antibodies, Miltenyi Inc., Auburn, Calif.) bone marrow (TDBM) on day 0, as described above, BUSULFEX (busulfan 20 mg/kg, i.p., Orphan Medical, Minnetonka, Minn.) on day −1, and 500 μg of hamster anti-mouse-CD40L mAb (MR1, BioExpress, Lebanon, N.H.) and 500 μg human CTLA4-Ig (Bristol-Myers Squibb, Princeton, N.J.), (for costimulation blockade) i.p. on days 0, 2, 4, 6 relative to the bone marrow transplant. Control mice received costimulation blockade and T cell depleted bone marrow, but no busulfan. The base-line hematological parameters were measured one week prior to transplant, and chimerism was tested two weeks, four weeks, and at monthly intervals after transplant.

Peripheral blood was analyzed by staining with fluorochrome-conjugated antibodies (anti-CD3, anti-CD5, anti-CD11b, anti-GR1, anti-B220, anti-H-2K^{d}, anti-H-2K^{b}, anti-Vβ5.1/5.2 (Pharmingen, Inc., San Diego, Calif.), anti-CD4, anti-CD8 (Caltag Laboratories, Burlingame, Calif.)) or immunoglobulin isotype controls (Pharmingen) followed by red blood cell lysis and washing with a whole blood lysis kit (R+D Systems, Minneapolis, Minn.).

Stained cells were analyzed either using WinList (Verity Software House Inc., Topsham, ME) or Cellquest (Beckton Dickinson, Mountain View, Calif.) software on either a FACScan or FACSCalibur flow cytometer (Beckton Dickinson). WBC chimerism was determined by staining with either donor (anti-H2K^{d}) or recipient (anti-H2K^{b}) antibodies and specific lineage markers, and analyzing by flow cytometry. Vβ deletion was determined by staining with Vβ5 antibodies and specific lineage markers, and analyzing by flow cytometry.

Complete Blood Counts were performed on a HEMAVET 1500 blood analyzer (1500 R series, CDC technologies, Oxford, Conn.). Reticulocyte counts were performed by flow cytometry of peripheral blood labeled with antibodies specific for red blood cells (anti-Ter-119, Pharmingen) and white blood cells (anti-CD45, Pharmingen) and a fluorescent label of RNA, Thiazole-Orange (Sigma Inc., St. Louis, Mo.). Reticulocyte counts were defined as the percent of peripheral blood cells that were Ter-119-Positive, Thiazole-Orange-positive, and CD45-negative. “Stress” reticulocytes were also analyzed by labeling with an antibody against the transferrin receptor (CD71, Pharmingen).

Red blood cell population half-life was determined by a pulsed biotinylation experiment performed essentially as previously described (Christian et al., Exp. Hematol., 24:82-88 (1996). Briefly, 50 mg/kg N-hydroxysuccidimide biotin (Calbiochem, San Diego, Calif.; initially dissolved at a concentration of 50 mg/ml in N, —N,-dimethylacetamide and diluted into 250 μl normal saline just prior to use) was injected (i.v.) into engrafted or naïve sickle animals. This produced a biotin pulse-label to the peripheral blood. Blood was obtained either from the retro-orbital venous plexus or through a tail-nick at regular intervals after biotinylation. The percentage of peripheral red blood cells that were biotinylated was determined by flow cytometry using fluorescent Strepdavidin-cychrome (Pharmingen) to identify biotinylated cells, and a fluorescent Ter-119-phycoerythrin antibody (Pharmingen) to identify red blood cells. The decay of biotinylation is directly related to the clearance of the biotinylated red blood cells from the peripheral circulation, and thus can be used to determine the half-life of the red blood cell population.

Plasma-membrane phosphatidylserine exposure was measured by the percentage of cells that were positive in Annexin-V (Pharmingen) binding assays. Annexin-V binding assays were performed by incubating 1×10⁶ peripheral blood cells with 5 μl Annexin-V and appropriate lineage-specific antibodies [in Annexin binding buffer (Pharmingen) for 30 minutes at room temperature. Cells were then washed once with Annexin binding buffer and analyzed by flow cytometry to determine the percentage Annexin-V-positive cells.

Red blood cell scramblase enzyme assays were performed essentially as previously described (Bevers et al., Biol. Chem., 8-9:973-986 (1998)). Briefly, 2×10⁶ peripheral blood cells were incubated with 3 nanomole/ml of the fluorescent phosphatidylcholine analog palmitoyl-C6-(N-(7-nitrobenz-2-oxa-1,3-diazol-4-yl)-phosphatidylcholine (NBD-PC; Avanti Polar Lipids, Birmingham, Ala.) in phosphate buffered saline containing 1 mM CaCl₂ for 30 minutes at 37 C. Cells were then cooled on ice and washed into buffer containing 10 mg/ml defatted bovine serum albumin (BSA) (Sigma, Inc.) to back-exchange non-internalized phospholipid. Cells were then incubated with appropriate lineage specific antibodies (Pharmingen) for 20 minutes at 40 C prior to analysis by flow cytometry.

Red blood cell chimerism was determined by differential hemoglobin electrophoresis of donor and recipient hemoglobin. Donor β-globin consists of murine “major” and “minor” β-globin isomers, which have different electrophoretic mobilities than recipient human sickle β-globin. Hemoglobin electrophoresis was performed on the Helena Titan III electrophoresis system (Helena laboratories, Beaumont, Tex.). Gels were scanned and percent donor or recipient hemoglobin was determined by densitometry using Kodak 1-D Image Analysis software (Kodak Inc., Rochester, NY).

CFSE assays were used to determine tolerance to donor antigen. Splenic and mesenteric lymph node cells were harvested from experimental mice. After red blood cell lysis and nylon wool passage, cells were incubated in 10 μM CFSE (Molecular Probes, Eugene, Oreg.). Irradiated (1800 rads) BALB/c, or C3H mice then intravenously received 1×10⁷-1×10⁹ CFSE-labeled cells. After 66-72 hours, splenocytes were harvested from the recipients, the red blood cells lysed, and the remaining cells stained with anti-CD4 and anti-CD8 antibodies, or isotype controls, and analyzed by flow cytometry, as described above.

To determine hematopoietic balance in recipient hematopoietic organs, mice were sacrificed and their splenocytes and bone marrow were harvested with conventional techniques. Hematopoietic balance was specified by determining the percent of bone marrow and spleen cells that were either red blood cells (Ter-119-positive, CD45-negative) reticulocytes (Ter-119-positive, CD45-negative, Thiazole-positive) or white blood cells (Ter-119-Negative, CD45-Positive).

Results

Creation of Stable Chimeras using Busulfan and Costimulation Blockade.

As indicated above, sickle mice were treated with a regimen that included busulfan (20 mg/kg) on day −1 (day 0 representing the day of bone marrow transplantation), transplantation with T cell depleted bone marrow from BALB/c mice on day 0, and co-stimulation blockade with 500 μg each of anti-CD40L and CTLA4-Ig on days 0, 2, 4, and 6. This group of animals is referred to as the “busulfan-treatment” group. The remaining animals received a control protocol including bone marrow transplantation and co-stimulation blockade, but no busulfan treatment. This protocol is well tolerated and non-myelosuppressive in multiple strains of mice.

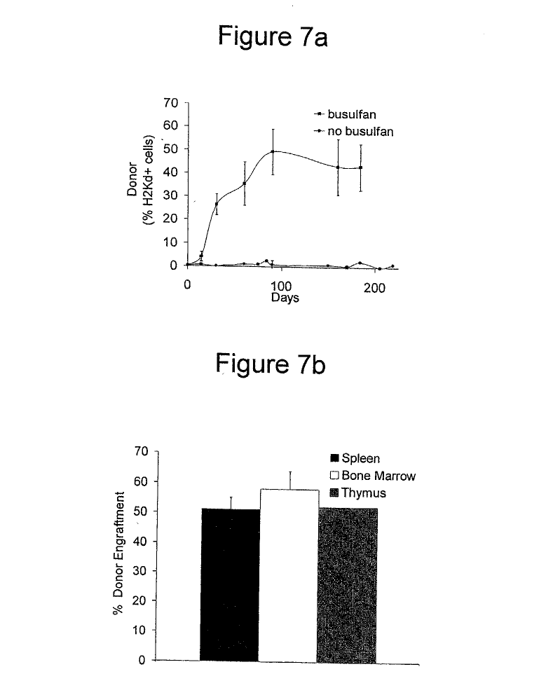

Of the busulfan-treated animals, 11/13 achieved multi-lineage white blood cell mixed chimerism (mean 43+10% on day 184) which peaked 3 months post-transplant and was stable for >150 days (FIG. 7A). Mice that received only costimulation blockade (but no busulfan) showed low-to-undetectable levels of peripheral white blood cell chimerism (<2.5%) throughout the same experimental period (FIG. 7A). The peripheral chimerism in the busulfan-treated animals was mirrored in their hematopoietic organs as shown in FIG. 7B (bone marrow (58%), spleen (51%) and thymus (52%)). Mice receiving bone marrow transplantation showed no signs of GVHD, i.e., their body weight remained stable and post-transplant necropsies showed normal histology of all organs.

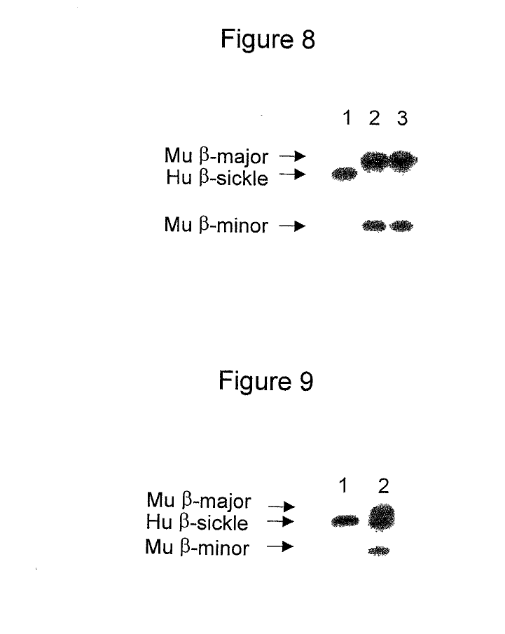

Peripheral white blood cell chimerism was comparable to results using busulfan and costimulation blockade in allogeneic wild-type and β-thalassemic mice (as described, supra), whereas peripheral red blood cell chimerism was strikingly higher in the sickle transplant recipients. Quantification of donor (normal BALB/c β-globin, major and minor-alleles) and recipient (human sickle-β-globin) hemoglobins separated by cellulose acetate electrophoresis showed 78-90% donor chimerism within two weeks that reached 100% by one month in all of the engrafted sickle mice (FIG. 8). Recipient mice originally possessed only human sickle β-globin (lane 1), while donor mice possessed the major and minor alleles of mouse β-globin (lane 2). Lane 3 shows a representative engrafted mouse with complete peripheral replacement with donor β-globin. Complete replacement of the peripheral blood with donor hemoglobin occurred within one month after transplant and was stable for the entire observation period (>150 days post transplant) in all engrafted mice (Hu=human. Mu=murine). The higher level of peripheral erythroid chimerism compared with white blood cell chimerism is consistent with the accumulation and enhanced survival of normal red blood cells in an environment with rapid turnover and degradation of sickle red blood cells.

Five of the nine control mice that were treated with only costimulation blockade (i.e., no busulfan) developed significant red cell chimerism (10-54%; FIG. 9) despite minimal white blood cell chimerism (<2.5%; FIG. 7A). Lane 1 of FIG. 9 shows a representative mouse without engraftment, with only human β-sickle hemoglobin. Lane 2 shows a representative mouse 3 months post-transplant with RBC chimerism, having both recipient (human β-sickle hemoglobin) and donor (mouse major and minor β-hemoglobin alleles).

Chimeric Mice are Specifically Tolerant to Donor Antigen.

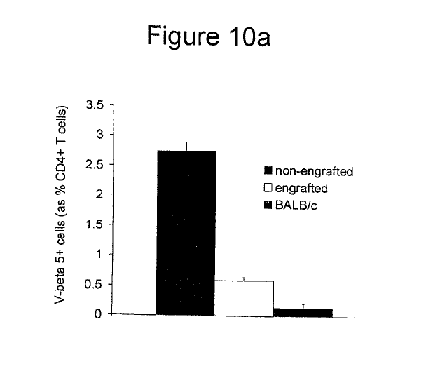

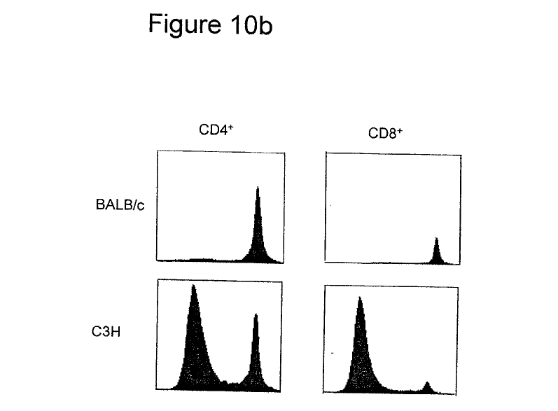

BALB/c mice express I-E and therefore delete Vβ5 bearing T cells, whereas sickle mice do not express I-E and specifically utilize Vβ5.1/2 on ˜2-3% of CD4⁺ T cells (Dyson et al. Nature, 349:531-549 (1991); Bill et al., J. Exp. Med., 169:1405-1419 (1989)). As anticipated, non-engrafted animals failed to delete donor reactive Vβ5⁺CD4⁺ T cells (FIG. 10A). In contrast, engrafted animals developed near complete deletion of CD4⁺Vβ5⁺ T cells by day 60. The percentage of Vβ8 bearing CD4⁺ T cells, normally expressed on 15-25% of BALB/c and Sickle CD4⁺ T cells was similar in all groups, indicating that the T cell deletion was donor specific in nature. These results suggest that the bone-marrow-derived I-E bearing donor cells influence the selection of the T cell repertoire in busulfan-treated mice, ultimately conferring robust long-term donor-specific tolerance.

Coincident with the long-term chimerism seen in the busulfan-treated animals, these animals also demonstrated specific tolerance to the allogeneic BALB/c bone marrow graft (FIG. 10B). A rigorous in vivo allo-proliferation (GVHD) assay utilizing CFSE dye was performed to test for the presence of lingering alloreactivity (Lyons et al., J. Immunol. Meth., 171:131-137 (1994)). T cells from engrafted and non-engrafted animals were harvested from spleens and mesenteric lymph nodes (T cells harvested from experimental animals >100 days after transplant). After labeling with 10 μM CFSE, T cells were transferred into recipient mice (BALB/c (donor) or C3H (third party)), previously supra-lethally irradiated with 1800 rads. Splenocytes were harvested 72 hours later and analyzed via flow cytometry.

While CD4⁺ and CD8⁺T cells from non-engrafted groups underwent extensive cell division in response to both BALB/c and third party (C3H) hosts, the histograms of FIG. 10B demonstrate that T cells from the engrafted mice generated no anti-donor proliferative response. As expected, clear proliferative responses to third party (C3H), were present in engrafted mice (FIG. 10B). These results confirm the specific absence of donor alloreactive CD4⁺ or CD8⁺ T cells capable of cell division in chimeric animals.

Tolerant animals, therefore show no proliferation to donor but a normal proliferative response to third party (C3H, H-2^{k}) grafts. Non-engrafted animals respond similarly to both donor and third party antigen in this GVHD model.

Chimeric Mice are Cured of Sickle Cell Disease.

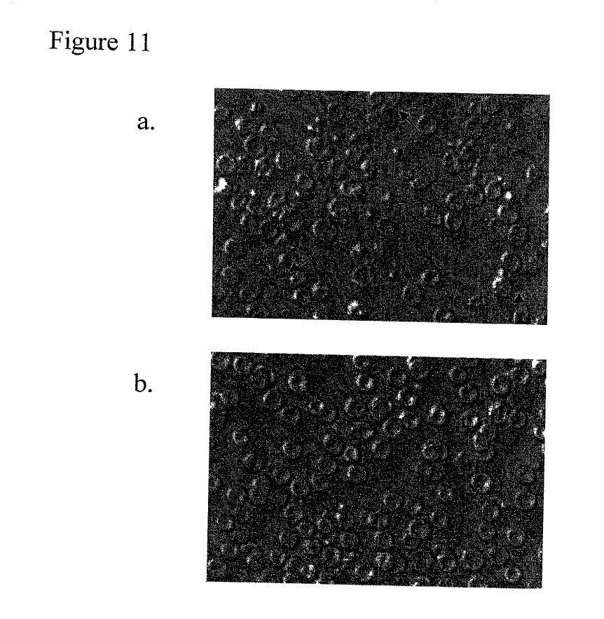

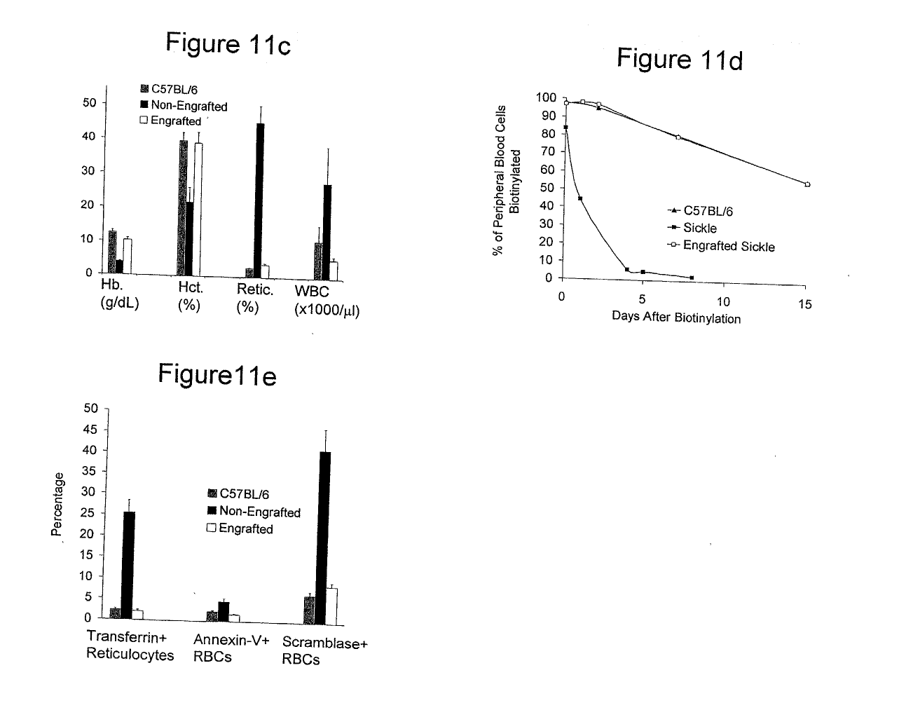

Engrafted sickle mice demonstrated a phenotypic cure of their sickle cell disease by a variety of parameters. As seen in FIGS. 11A and 11B, a striking absence of irreversibly sickled cells in peripheral blood smears occurred after busulfan-conditioned transplantation. Arrows in FIG. 11A point to representative sickled cells in the untreated blood. Engrafted mice also demonstrated normalization of their hematological abnormalities (FIG. 11C) including hemoglobin (Hb; 4.5 g/dL corrected to 10 g/dL), hematocrit (Hct; 16% corrected to 40%), and peripheral thiazole-positive reticulocyte % (Retic.; 49% corrected to 3.5%), consistent with a reversal of their hemolytic anemia. Furthermore, the abnormally elevated white blood cells (WBCs) seen in naïve sickle mice was corrected in engrafted mice (20,000/μl to 5100/μl). Normalization of hematological parameters was stable for the entire experimental period (>150 days). Shown are the mean +/−sem for C57BL/6 controls, non-engrafted mice (black), and engrafted mice (white) in a representative experiment performed three months after transplant.

The health of the newly emerging chimeric red cells was assessed by three physiologic markers. First, red blood cell population half-life was determined through a pulsed biotinylation experiment, as described in Christian et al., Exp. Hematol., 24:82-88 (1996) (FIG. 11D). Untreated and engrafted sickle mice were intravenously injected with N-hydroxysuccidimide-biotin to label the peripheral blood with biotin. Red blood cell (identified as Ter-119-positive, CD45-negative, biotinylated cells) half-life was determined by the decay of the biotinylated red blood cells over time by flow cytometry. Red blood cells from the naïve sickle animals (filled squares) had exceedingly short peripheral half-lives (0.8 days) compared with normal control C57BL/6 mice (filled triangles; half-life 18 days). Engrafted animals (open squares) had a red blood cell half-life indistinguishable from that of normal mice, consistent with replacement of the diseased red cell compartment with normal red blood cells. Second, the production of transferrin-positive “stress” reticulocytes in engrafted and non-engrafted mice was measured. These cells are an indication of over-active erythropoiesis and are thought to contribute to the increased adhesion of sickle reticulocytes in the microvasculature (Serke et al., Br. J. Haematol., 81:432-439 (1992); Swerlick et al., Blood, 82:1891-1899 (1993); and Joneckis et al., Blood, 82:3548-3555 (1993)). FIG. 11E shows that the percent of these cells decreases from 27% to 3% in engrafted mice, consistent with normalization of red cell turnover in these animals. Third, plasma membrane phosphatidylserine exposure, which is known to be increased in sickle red cells, was examined (Wood et al., Blood, 8:1873-1880 (1996); Kuypers et al., Blood, 87:1179-1187 (1996)). Phosphatidylserine is thought to contribute to increased clearance of these cells by macrophages and monocytes and may also contribute to abnormal endothelial adhesion (Closse et al., Br. J. Haematol., 107:300-302 (1999). Two assays were used. One assay measured Annexin-V binding, which measures exposed phosphatidylserine residues directly (Vermes et al., J. Immunol. Meth., 184:39-51 (1995), and the second assay measured NBD-PC internalization, which measures the scramblase enzyme that leads to phosphatidylserine exposure on the plasma membrane (Frasch et al., J. Biol. Chem., 275:23065-23073 (2000); Bevers et al., Biol. Chem., 8-9:973-986 (1998); and Bratton et al., J. Biol. Chem., 272:26159-26165 (1997)). FIG. 11E shows that sickle mice consistently show a high phosphatidylserine exposure prior to transplant (measured by Annexin-V binding), but engrafted mice demonstrate a significant decrease in this phosphatidylserine-exposure. FIG. 11E also shows that a dramatic decrease in the number of red blood cells with active scramblase occurs after engraftment, consistent with the decline in phosphatidylserine exposure described above.

The Spleens in the Engrafted Mice Also Exhibit Signs of Reversal of the Sickle Phenotype.

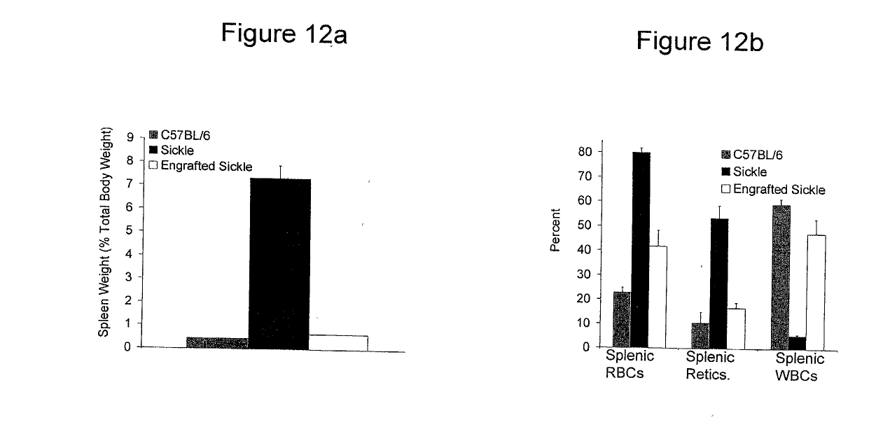

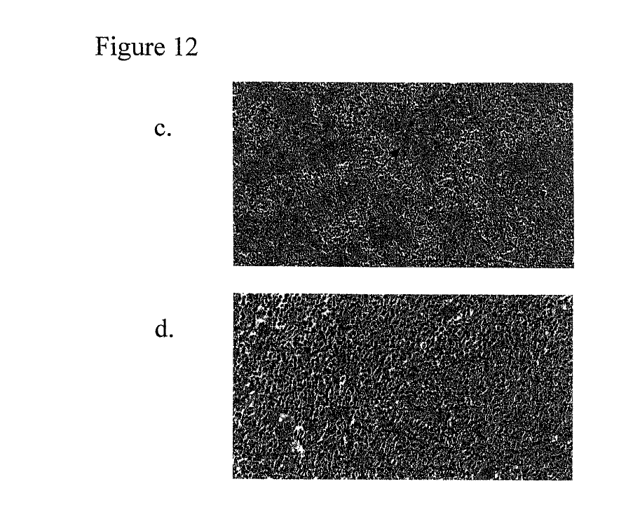

One of the hallmarks of murine sickle cell pathophysiology is the dramatic increase in spleen size compared to normal animals (Pastzy et al., Science, 278:876-878 (1997)). This is related to the immense requirement for splenic hematopoiesis in order to replenish the rapid destruction of peripheral sickle red blood cells. As shown in FIG. 12A, the spleen undergoes a significant decrease in size in engrafted mice (from 7.3% total body weight in naive sickle mice to 0.6% total body weight in engrafted mice measured 3 months after transplantation). FIG. 12B shows that while the spleen functions as a largely erythropoietic organ in untreated sickle mice, it undergoes a re-programming in the engrafted cohort, and resumes a more normal balance between white and red cell hematopoiesis. FIGS. 12C and 12D show a histological comparison of the spleens from naive and engrafted mice, showing that engrafted mice have a resolution of the characteristic hyperactive hematopoiesis and red cell sequestration characteristic of sickle cell disease. FIG. 12C shows that the spleen from a sickle animal is highly abnormal with pooling of sickled red blood cells and areas of increased hematopoiesis. For example, the arrow points to a representative red blood cell pool. No red blood cell pooling is evident in the engrafted mouse.

Renal Histology is Normal in Engrafted Mice.

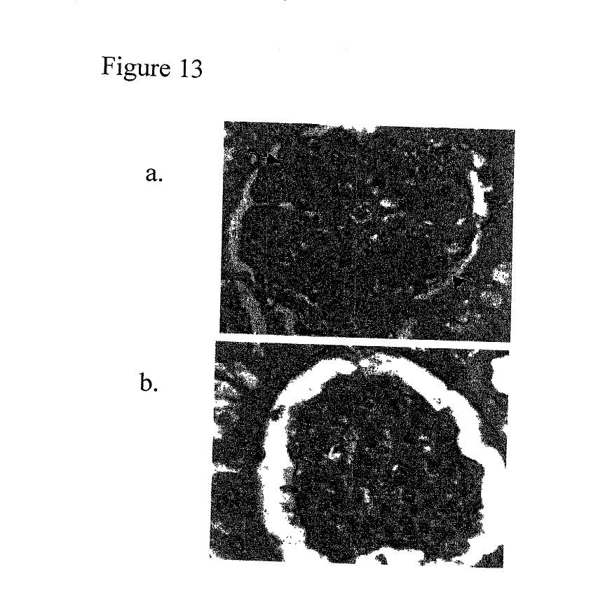

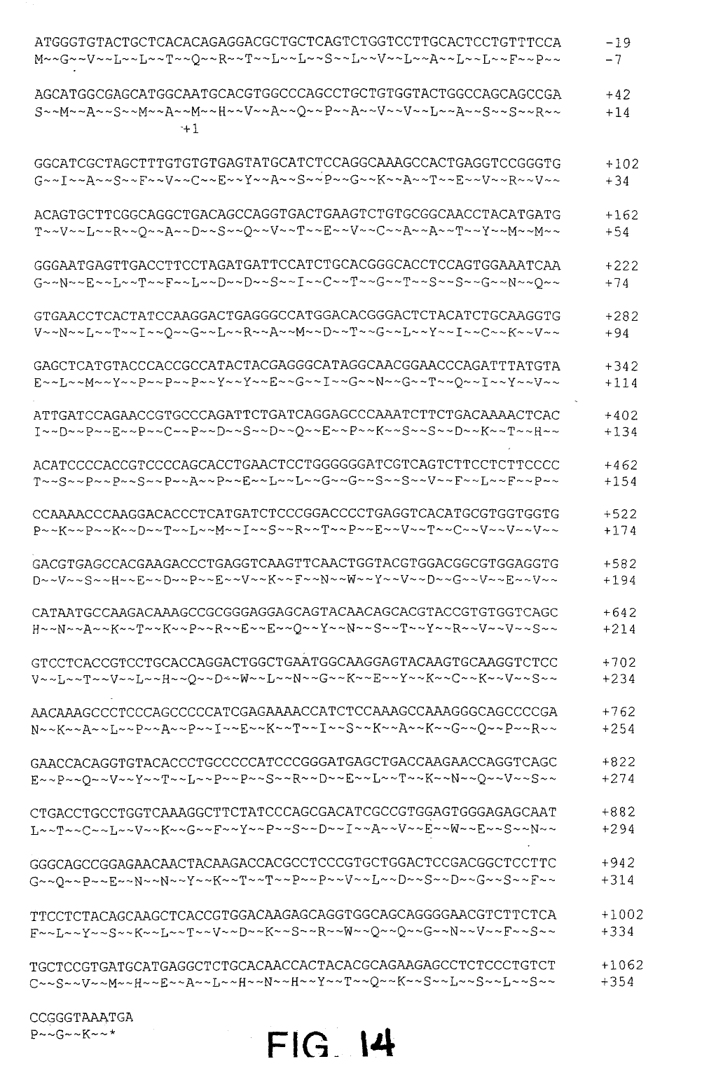

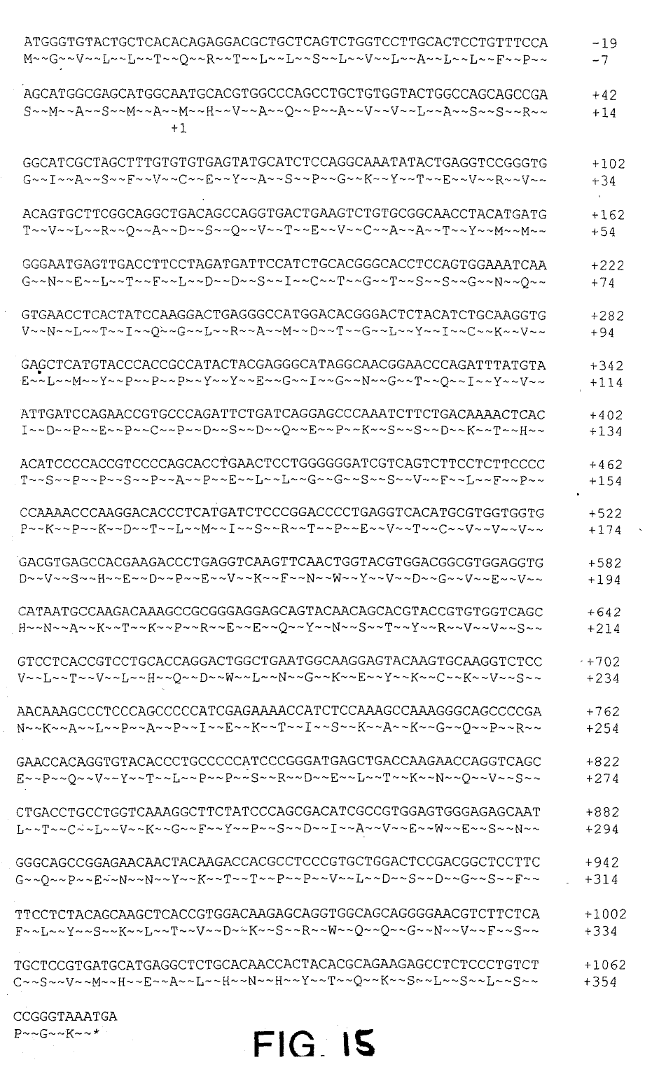

In addition to the defects observed in both the peripheral blood and the hematopoietic organs, sickle mice also demonstrate solid organ pathology similar to that seen in patients with sickle cell disease (Pastzy et al., Science, 278:876-878 (1997)). As in the original description of this murine sickle cell disease model (Pastzy et al., Science, 278:876-878 (1997)), we have noted pathologic changes in many organs including the kidney, liver, lung, and heart in untreated sickle animals. To determine the effect of bone marrow transplantation on organ structure and histology, necropsies were performed on naive and engrafted animals and tissues were prepared for histologic analysis. Engrafted animals had normal histology of all organs tested including the kidney, liver, heart and lungs. Representative of the histological normalization that occurred in these animals, FIGS. 13A and 13B shows a comparison of renal histology in untreated and engrafted mice. FIG. 13A shows the membranoproliferative glomerulonephritis consistently observed in untreated sickle mice. The arrow points to thickened glomerular membrane, and the arrowhead points to narrowed glomerular space. FIG. 13B shows that engrafted animals had normal renal histology including normalization of glomerular capsular space and glomerular membrane thickness.

### Example 8

The Following Provides a Description of the Methods Used to Generate the Nucleotide Sequences Encoding the CTLA4 Molecules of the Invention.

A CTLA4Ig encoding plasmid was first constructed, and shown to express CTLA4Ig molecules as described in U.S. Pat. Nos. 5,434,131, 5,885,579 and 5,851,795. Then single-site mutant molecules (e.g., L104EIg) were generated from the CTLA4Ig encoding sequence, expressed and tested for binding kinetics for various B7 molecules. The L104EIg nucleotide sequence (as included in the sequence shown in FIG. 14) was used as a template to generate the double-site CTLA4 mutant sequences (as included in the sequences shown in FIGS. 15-18) which were expressed as proteins and tested for binding kinetics. The double-site CTLA4 mutant sequences include: L104EA29YIg, L104EA29LIg, L104EA29TIg, and L104EA29WIg. Triple-site mutants were also generated.

CTLA4Ig Construction

A genetic construct encoding CTLA4Ig comprising the extracellular domain of CTLA4 and an IgCgammal domain was constructed as described in U.S. Pat. Nos. 5,434,131, 5,844,095 and 5,851,795, the contents of which are incorporated by reference herein. The extracellular domain of the CTLA4 gene was cloned by PCR using synthetic oligonucleotides corresponding to the published sequence (Dariavach et al., Eur. Journ. Immunol. 18:1901-1905 (1988)).

Because a signal peptide for CTLA4 was not identified in the CTLA4 gene, the N-terminus of the predicted sequence of CTLA4 was fused to the signal peptide of oncostatin M (Malik et al., Mol. and Cell. Biol. 9:2847 (1989)) in two steps using overlapping oligonucleotides. For the first step, the oligonucleotide, CTCAGTCTGGTCCTTGCACTCCTGTTTCCAAGCATGGCGAGCATGGCAATGCA CGTGGCCCAGCC (SEQ ID NO.: 16) (which encoded the C terminal 15 amino acids from the oncostatin M signal peptide fused to the N terminal 7 amino acids of CTLA4) was used as forward primer, and TTTGGGCTCCTGATCAGAATCTGGGCACGGTTG (SEQ ID NO.: 17) (encoding amino acid residues 119-125 of the amino acid sequence encoding CTLA4 receptor and containing a Bcl I restriction enzyme site) as reverse primer. The template for this step was cDNA synthesized from 1 micro g of total RNA from H38 cells (an HTLV II infected T-cell leukemic cell line provided by Drs. Salahudin and Gallo, NCI, Bethesda, Md.). A portion of the PCR product from the first step was reamplified, using an overlapping forward primer, encoding the N terminal portion of the oncostatin M signal peptide and containing a Hind III restriction endonuclease site, CTAGCCACTGAAGCTTCACCAATGGGTGTACTGCTCACACAGAGGACGCTGC TCAGTCTGGTCCTTGCACTC (SEQ ID NO.: 18) and the same reverse primer. The product of the PCR reaction was digested with Hind III and Bcl I and ligated together with a Bcl 1/Xba I cleaved cDNA fragment encoding the amino acid sequences corresponding to the hinge, CH2 and CH3 regions of IgC(gamma)1 into the Hind III/Xba I cleaved expression vector, CDM8 or Hind III/Xba I cleaved expression vector piLN (also known as πLN).

DNA encoding the amino acid sequence corresponding to CTLA4Ig has been deposited with the ATCC under the Budapest Treaty on May 31, 1991, and has been accorded ATCC accession number 68629.

CTLA4Ig Codon Based Mutagenesis

A mutagenesis and screening strategy was developed to identify mutant CTLA4Ig molecules that had slower rates of dissociation (“off” rates) from CD80 and/or CD86 molecules i.e. improved binding ability. In this embodiment, mutations were carried out in and/or about the residues in the CDR-1, CDR-2 (also known as the C′ strand) and/or CDR-3 regions of the extracellular domain of CTLA4 (as described in U.S. Patents U.S. Pat. Nos. 6,090,914, 5,773,253 and 5,844,095; in copending U.S. Patent Application Serial No. 60/214,065; and by Peach, R. J., et al J Exp Med 1994 180:2049-2058. A CDR-like region encompasses the each CDR region and extends, by several amino acids, upstream and/or downstream of the CDR motif). These sites were chosen based on studies of chimeric CD28/CTLA4 fusion proteins (Peach et al., J. Exp. Med., 1994, 180:2049-2058), and on a model predicting which amino acid residue side chains would be solvent exposed, and a lack of amino acid residue identity or homology at certain positions between CD28 and CTLA4. Also, any residue which is spatially in close proximity (5 to 20 Angstrom Units) to the identified residues is considered part of the present invention.

To synthesize and screen soluble CTLA4 mutant molecules with altered affinities for a B7 molecule (e.g. CD80, CD86), a two-step strategy was adopted. The experiments entailed first generating a library of mutations at a specific codon of an extracellular portion of CTLA4 and then screening these by BIAcore analysis to identify mutants with altered reactivity to B7. The Biacore assay system (Pharmacia, Piscataway, N.J.) uses a surface plasmon resonance detector system that essentially involves covalent binding of either CD80Ig or CD86Ig to a dextran-coated sensor chip which is located in a detector. The test molecule can then be injected into the chamber containing the sensor chip and the amount of complementary protein that binds can be assessed based on the change in molecular mass which is physically associated with the dextran-coated side of the sensor chip; the change in molecular mass can be measured by the detector system.

Specifically, single-site mutant nucleotide sequences were generated using non-mutated (e.g., wild-type) DNA encoding CTLA4Ig (U.S. Pat. Nos. 5,434,131, 5,844,095; 5,851,795; and 5,885,796; ATCC Accession No. 68629) as a template. Mutagenic oligonucleotide PCR primers were designed for random mutagenesis of a specific codon by allowing any base at positions 1 and 2 of the codon, but only guanine or thymine at position 3 (XXG/T or also noted as NNG/T). In this manner, a specific codon encoding an amino acid could be randomly mutated to code for each of the 20 amino acids. In that regard, XXG/T mutagenesis yields 32 potential codons encoding each of the 20 amino acids. PCR products encoding mutations in close proximity to the CDR3-like loop of CTLA4Ig (MYPPPY), were digested with SacI/XbaI and subcloned into similarly cut CTLA4Ig (as included in FIG. 20) πLN expression vector. This method was used to generate the single-site CTLA4 mutant molecule L104EIg (as included in FIG. 14).

For mutagenesis in proximity to the CDR-1-like loop of CTLA4Ig, a silent NheI restriction site was first introduced 5′ to this loop, by PCR primer-directed mutagenesis. PCR products were digested with NheI/XbaI and subcloned into similarly cut CTLA4Ig or L104EIg expression vectors. This method was used to generate the double-site CTLA4 mutant molecule L104EA29YIg (as included in FIG. 15). In particular, the nucleic acid molecule encoding the single-site CTLA4 mutant molecule, L104EIg, was used as a template to generate the double-site CTLA4 mutant molecule, L104EA29YIg.

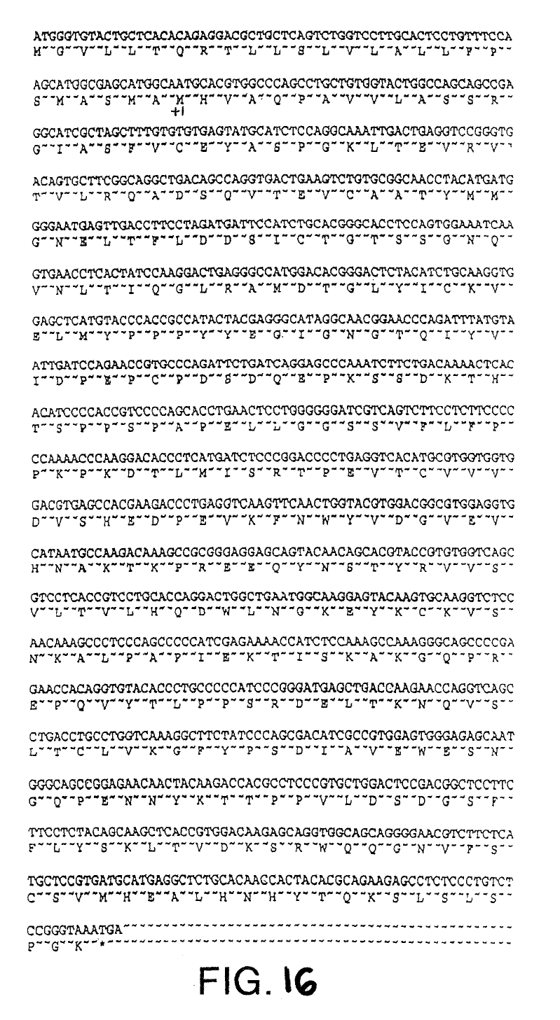

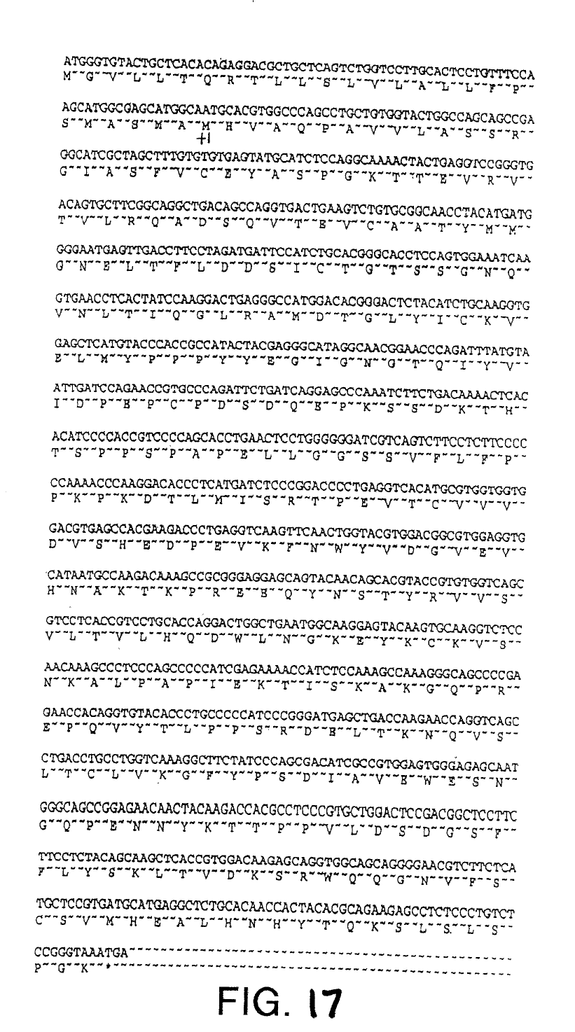

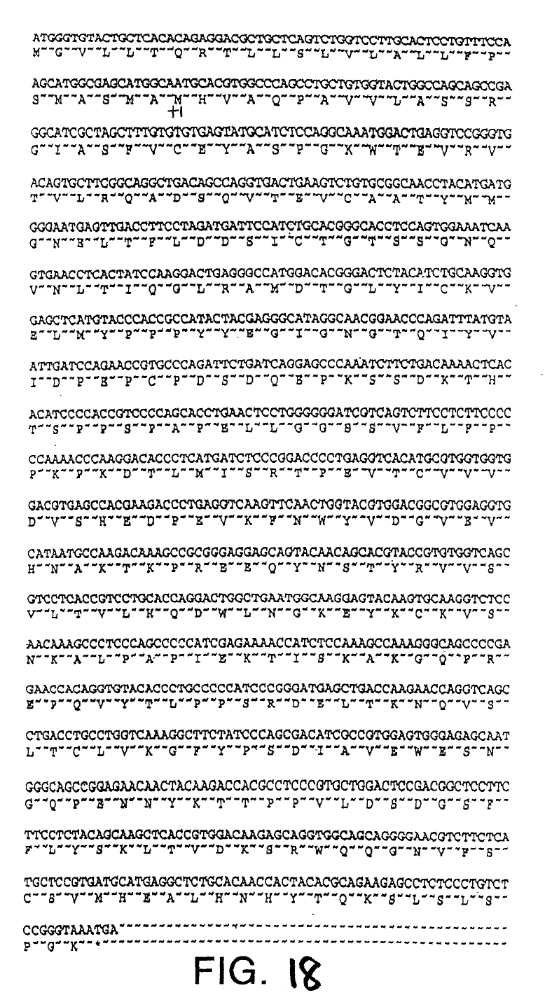

The double-site mutant nucleotide sequences encoding CTLA4 mutant molecules, such as L104EA29YIg (deposited on Jun. 19, 2000 with the American Type Culture Collection (ATCC), 10801 University Blvd., Manassas, Va. 20110-2209 and accorded ATCC accession number PTA-2104), were generated by repeating the mutagenesis procedure described above using L104EIg as a template. This method was used to generate numerous double-site mutants nucleotide sequences such as those encoding CTLA4 molecules L104EA29YIg (as included in the sequence shown in FIG. 15), L104EA29LIg (as included in the sequence shown in FIG. 16), L104EA29TIg (as included in the sequence shown in FIG. 17), and L104EA29WIg (as included in the sequence shown in FIG. 18). Triple-site mutants, such as those encoding L104EA29YS25KIg, L104EA29YS25NIg and L104EA29YS25RIg, were also generated

The soluble CTLA4 molecules were expressed from the nucleotide sequences and used in the phase II clinical studies described in Example 3, infra.

As those skilled-in-the-art will appreciate, replication of nucleic acid sequences, especially by PCR amplification, easily introduces base changes into DNA strands. However, nucleotide changes do not necessarily translate into amino acid changes as some codons redundantly encode the same amino acid. Any changes of nucleotide from the original or wildtype sequence, silent (i.e. causing no change in the translated amino acid) or otherwise, while not explicitly described herein, are encompassed within the scope of the invention.

### Example 9

The following example provides a description of the screening methods used to identify the single- and double-site mutant CTLA polypeptides, expressed from the constructs described in Example 8, that exhibited a higher binding avidity for B7 molecules, compared to non-mutated CTLA4Ig molecules.

Current in vitro and in vivo studies indicate that CTLA4Ig by itself is unable to completely block the priming of antigen specific activated T cells. In vitro studies with CTLA4Ig and either monoclonal antibody specific for CD80 or CD86 measuring inhibition of T cell proliferation indicate that anti-CD80 monoclonal antibody did not augment CTLA4Ig inhibition. However, anti-CD86 monoclonal antibody did augment the inhibition, indicating that CTLA4Ig was not as effective at blocking CD86 interactions. These data support earlier findings by Linsley et al. (Immunity, (1994), 1:793-801) showing inhibition of CD80-mediated cellular responses required approximately 100 fold lower CTLA4Ig concentrations than for CD86-mediated responses. Based on these findings, it was surmised that soluble CTLA4 mutant molecules having a higher avidity for CD86 than wild type CTLA4 should be better able to block the priming of antigen specific activated cells than CTLA4Ig.

To this end, the soluble CTLA4 mutant molecules described in Example 8 above were screened using a novel screening procedure to identify several mutations in the extracellular domain of CTLA4 that improve binding avidity for CD80 and CD86. This screening strategy provided an effective method to directly identify mutants with apparently slower “off” rates without the need for protein purification or quantitation since “off” rate determination is concentration independent (O'Shannessy et al., (1993) Anal. Biochem., 212:457-468).

COS cells were transfected with individual miniprep purified plasmid DNA and propagated for several days. Three day conditioned culture media was applied to BIAcore biosensor chips (Pharmacia Biotech AB, Uppsala, Sweden) coated with soluble CD80Ig or CD86Ig. The specific binding and dissociation of mutant proteins was measured by surface plasmon resonance (O'Shannessy, D. J., et al., 1997 Anal. Biochem. 212:457-468). All experiments were run on BIAcore™ or BIAcore™ 2000 biosensors at 25° C. Ligands were immobilized on research grade NCM5 sensor chips (Pharmacia) using standard N-ethyl-N′-(dimethylaminopropyl) carbodiimidN-hydroxysuccinimide coupling (Johnsson, B., et al. (1991) Anal. Biochem. 198: 268-277; Khilko, S. N., et al.(1993) J. Biol. Chem 268:5425-15434).

Screening Method

COS cells grown in 24 well tissue culture plates were transiently transfected with mutant CTLA4Ig. Culture media containing secreted soluble mutant CTLA4Ig was collected 3 days later.

Conditioned COS cell culture media was allowed to flow over BIAcore biosensor chips derivitized with CD86Ig or CD80Ig (as described in Greene et al., 1996 J. Biol. Chem. 271:26762-26771), and mutant molecules were identified with off-rates slower than that observed for wild type CTLA4Ig. The DNAs corresponding to selected media samples were sequenced and more DNA prepared to perform larger scale COS cell transient transfection, from which CTLA4Ig mutant protein was prepared following protein A purification of culture media.

BIAcore analysis conditions and equilibrium binding data analysis were performed as described in J. Greene et al. 1996 J. Biol. Chem. 271:26762-26771 and in U.S. patent application Ser. Nos. 09/579,927, and 60/214,065 which are herein incorporated by reference.

BIAcore Data Analysis

Senosorgram baselines were normalized to zero response units (RU) prior to analysis. Samples were run over mock-derivatized flow cells to determine background RU values due to bulk refractive index differences between solutions. Equilibrium dissociation constants (K_{d}) were calculated from plots of R_{eq} versus C, where R_{eq} is the steady-state response minus the response on a mock-derivatized chip, and C is the molar concentration of analyte. Binding curves were analyzed using commercial nonlinear curve-fitting software (Prism, GraphPAD Software).

Experimental data were first fit to a model for a single ligand binding to a single receptor (1-site model, i.e., a simple langmuir system, A+B AB), and equilibrium association constants (K_{d}=[A]·[B]\[AB]) were calculated from the equation R=Rₘₐₓ·C/(K_{d}+C). Subsequently, data were fit to the simplest two-site model of ligand binding (i.e., to a receptor having two non-interacting independent binding sites as described by the equation R=Rₘₐₓ₁·C\(K_{d1}+C)+Rₘₐₓ₂·C\(K_{d2}+C).

The goodness-of-fits of these two models were analyzed visually by comparison with experimental data and statistically by an F test of the sums-of-squares. The simpler one-site model was chosen as the best fit, unless the two-site model fit significantly better (p<0.1).

Association and disassociation analyses were performed using BIA evaluation 2.1 Software Pharmacia). Association rate constants kₒₙ were calculated in two ways, assuming both homogenous single-site interactions and parallel two-site interactions. For single-site interactions, kₒₙ values were calculated according to the equation Rₜ=R_{eq}(1−exp^{-ks(t-t}₀), where Rₜ is a response at a given time, t; R_{eq} is the steady-state response; to is the time at the start of the injection; and kₛ=dR/dt=kₒₙ·Ck_{off}, where C is a concentration of analyte, calculated in terms of monomeric binding sites. For two-site interactions kₒₙ values were calculated according to the equation Rₜ=R_{eq1}(1−exp^{-ks1(t-t}₀)+R_{eq2}(1−exp^{ks2(t-t}₀). For each model, the values of kₒₙ were determined from the calculated slope (to about 70% maximal association) of plots of kₛ versus C.

Dissociation data were analyzed according to one site (AB=A+B) or two site (AiBj=Ai+Bj) models, and rate constants (k_{off}) were calculated from best fit curves. The binding site model was used except when the residuals were greater than machine background (2-10RU, according to machine), in which case the two-binding site model was employed. Half-times of receptor occupancy were calculated using the relationship t_{1/2}=0.693/k_{off}.

Flow Cytometry

Murine mAb L307.4 (anti-CD80) was purchased from Becton Dickinson (San Jose, Calif.) and IT2.2 (anti-B7-0 [also known as CD86]), from Pharmingen (San Diego, Calif.). For immunostaining, CD80-positive and/or CD86-positive CHO cells were removed from their culture vessels by incubation in phosphate-buffered saline (PBS) containing 10 mM EDTA. CHO cells (1-10×10⁵) were first incubated with mAbs or immunoglobulin fusion proteins in DMEM containing 10% fetal bovine serum (FBS), then washed and incubated with fluorescein isothiocyanate-conjugated goat anti-mouse or anti-human immunoglobulin second step reagents (Tago, Burlingame, Calif.). Cells were given a final wash and analyzed on a FACScan (Becton Dickinson).

SDS-PAGE and Size Exclusion Chromatography

SDS-PAGE was performed on Tris/glycine 4-20% acrylamide gels (Novex, San Diego, Calif.). Analytical gels were stained with Coomassie Blue, and images of wet gels were obtained by digital scanning. CTLA4Ig (25 μg) and L104EA29YIg (25 μg) were analyzed by size exclusion chromatography using a TSK-GEL G300 SW_{XL} column (7.8×300 mm, Tosohaas, Montgomeryville, Pa.) equilibrated in phosphate buffered saline containing 0.02% NAN₃ at a flow rate of 1.0 ml/min.

CTLA4X_{C120S} and L104EA29YX_{C120S}.

Single chain CTLA4X_{C120S} was prepared as previously described (Linsley et al., (1995) J. Biol. Chem., 270:15417-15424). Briefly, an oncostatin M CTLA4 (OMCTLA4) expression plasmid was used as a template, the forward primer, GAGGTGATAAAGCTTCACCAATGGGTGTACTGCTCACACAG (SEQ ID NO.: 19) was chosen to match sequences in the vector; and the reverse primer, GTGGTGTATTGGTCTAGATCAATCAGAATCTGGGCACGGTTC (SEQ ID NO.: 20) corresponded to the last seven amino acids (i.e. amino acids 118-124) in the extracellular domain of CTLA4, and contained a restriction enzyme site, and a stop codon (TGA). The reverse primer specified a C120S (cysteine to serine at position 120) mutation. In particular, the nucleotide sequence GCA (nucleotides 34-36) of the reverse primer shown above is replaced with one of the following nucleotide sequences: AGA, GGA, TGA, CGA, ACT, or GCT. As persons skilled in the art will understand, the nucleotide sequence GCA is a reversed complementary sequence of the codon TGC for cysteine. Similarly, the nucleotide sequences AGA, GGA, TGA, CGA, ACT, or GCT are the reversed complementary sequences of the codons for serine. Polymerase chain reaction products were digested with HindIII/XbaI and directionally subcloned into the expression vector πLN (Bristol-Myers Squibb Company, Princeton, N.J.). L104EA29YX_{C120S} was prepared in an identical manner. Each construct was verified by DNA sequencing.

Identification and Biochemical Characterization of High Avidity Mutants

Twenty four amino acids were chosen for mutagenesis and the resulting ˜2300 mutant proteins assayed for CD86Ig binding by surface plasmon resonance (SPR; as described, supra). The predominant effects of mutagenesis at each site are summarized in Table II, infra. Random mutagenesis of some amino acids in the CDR-1 region (S25-R33) apparently did not alter ligand binding. Mutagenesis of E31 and R33 and residues M97-Y102 apparently resulted in reduced ligand binding. Mutagenesis of residues, S25, A29, and T30, K93, L96, Y103, L104, and G105, resulted in proteins with slow “on” and/or slow “off” rates. These results confirm previous findings that residues in the CDR-1 (S25-R33) region, and residues in or near M97-Y102 influence ligand binding (Peach et al., (1994) J. Exp. Med., 180:2049-2058).

Mutagenesis of sites S25, T30, K93, L96, Y103, and G105 resulted in the identification of some mutant proteins that had slower “off” rates from CD86Ig. However, in these instances, the slow “off” rate was compromised by a slow “on” rate that resulted in mutant proteins with an overall avidity for CD86Ig that was apparently similar to that seen with wild type CTLA4Ig. In addition, mutagenesis of K93 resulted in significant aggregation that may have been responsible for the kinetic changes observed.

Random mutagenesis of L104 followed by COS cell transfection and screening by SPR of culture media samples over immobilized CD86Ig yielded six media samples containing mutant proteins with approximately 2-fold slower “off” rates than wild type CTLA4Ig. When the corresponding cDNA of these mutants were sequenced, each was found to encode a leucine to glutamic acid mutation (L104E). Apparently, substitution of leucine 104 to aspartic acid (L104D) did not affect CD86Ig binding.

Mutagenesis was then repeated at each site listed in Table II, this time using L104E as the PCR template instead of wild type CTLA4Ig, as described above. SPR analysis, again using immobilized CD86Ig, identified six culture media samples from mutagenesis of alanine 29 with proteins having approximately 4-fold slower “off” rates than wild type CTLA4Ig. The two slowest were tyrosine substitutions (L104EA29Y), two were leucine (L104EA29L), one was tryptophan (L104EA29W), and one was threonine (L104EA29T). Apparently, no slow “off” rate mutants were identified when alanine 29 was randomly mutated, alone, in wild type CTLA4Ig.

The relative molecular mass and state of aggregation of purified L104E and L104EA29YIg was assessed by SDS-PAGE and size exclusion chromatography. L104EA29YIg (˜1 μg; lane 3) and L104EIg (˜1 μg; lane 2) apparently had the same electrophoretic mobility as CTLA4Ig (˜1 μg; lane 1) under reducing (˜50 kDa; +βME; plus 2-mercaptoethanol) and non-reducing (˜100 kDa; −βME) conditions (FIG. 21A). Size exclusion chromatography demonstrated that L104EA29YIg (FIG. 21C) apparently had the same mobility as dimeric CTLA4Ig (FIG. 21B). The major peaks represent protein dimer while the faster eluting minor peak in FIG. 21B represents higher molecular weight aggregates. Approximately 5.0% of CTLA4Ig was present as higher molecular weight aggregates but there was no evidence of aggregation of L104EA29YIg or L104EIg. Therefore, the stronger binding to CD86Ig seen with L104EIg and L104EA29YIg could not be attributed to aggregation induced by mutagenesis.

Equilibrium and Kinetic Binding Analysis

Equilibrium and kinetic binding analysis was performed on protein A purified CTLA4Ig, L104EIg, and L104EA29YIg using surface plasmon resonance (SPR). The results are shown in Table I, infra. Observed equilibrium dissociation constants (K_{d}; Table 1) were calculated from binding curves generated over a range of concentrations (5.0-200 nM). L104EA29YIg binds more strongly to CD86Ig than does L104EIg or CTLA4Ig. The lower K_{d} of L104EA29YIg (3.21 nM) than L104EIg (6.06 nM) or CTLA4Ig (13.9 nM) indicates higher binding avidity of L104EA29YIg to CD86Ig. The lower K_{d} of L104EA29YIg (3.66 nM) than L104EIg (4.47 nM) or CTLA4Ig (6.51 nM) indicates higher binding avidity of L104EA29YIg to CD80Ig.

Kinetic binding analysis revealed that the comparative “on” rates for CTLA4Ig, L104EIg, and L104EA29YIg binding to CD80 were similar, as were the “on” rates for CD86Ig (Table I). However, “off” rates for these molecules were not equivalent (Table I). Compared to CTLA4Ig, L104EA29YIg had approximately 2-fold slower “off” rate from CD80Ig, and approximately 4-fold slower “off” rate from CD86Ig. L104E had “off” rates intermediate between L104EA29YIg and CTLA4Ig. Since the introduction of these mutations did not significantly affect “on” rates, the increase in avidity for CD80Ig and CD86Ig observed with L104EA29YIg was likely primarily due to a decrease in “off” rates.

To determine whether the increase in avidity of L104EA29YIg for CD86Ig and CD80Ig was due to the mutations affecting the way each monomer associated as a dimer, or whether there were avidity enhancing structural changes introduced into each monomer, single chain constructs of CTLA4 and L104EA29Y extracellular domains were prepared following mutagenesis of cysteine 120 to serine as described supra, and by Linsley et al., (1995) J. Biol. Chem., 270:15417-15424 (84). The purified proteins CTLA4X_{C120S} and L104EA29YX_{C120S} were shown to be monomeric by gel permeation chromatography (Linsley et al., (1995), supra), before their ligand binding properties were analyzed by SPR. Results showed that binding affinity of both monomeric proteins for CD86Ig was approximately 35-80-fold less than that seen for their respective dimers (Table I). This supports previously published data establishing that dimerization of CTLA4 was required for high avidity ligand binding (Greene et al., (1996) J. Biol. Chem., 271:26762-26771).

L104EA29YX_{C120S} bound with approximately 2-fold higher affinity than CTLA4X_{C120S} to both CD80Ig and CD86Ig. The increased affinity was due to approximately 3-fold slower rate of dissociation from both ligands. Therefore, stronger ligand binding by L104EA29Y was most likely due to avidity enhancing structural changes that had been introduced into each monomeric chain rather than alterations in which the molecule dimerized.

Location and Structural Analysis of Avidity Enhancing Mutations

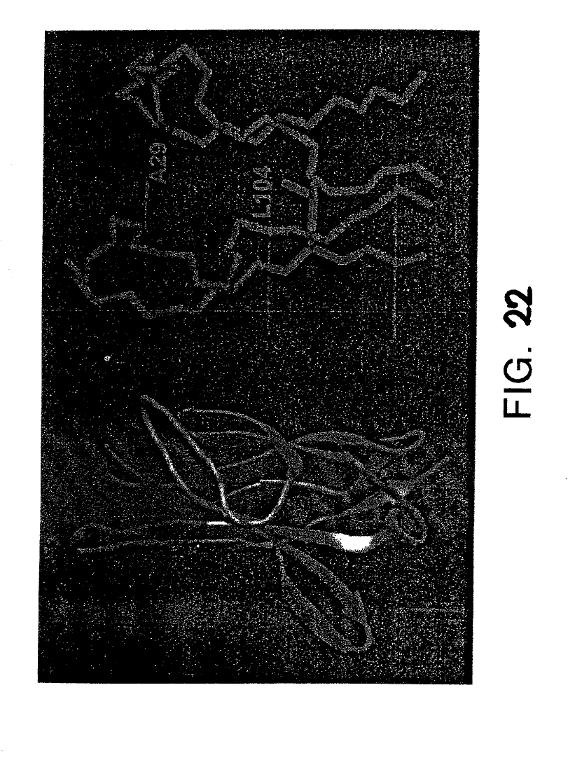

The solution structure of the extracellular IgV-like domain of CTLA4 has recently been determined by NMR spectroscopy (Metzler et al., (1997) Nature Struct. Biol., 4:527-531). This allowed accurate location of leucine 104 and alanine 29 in the three dimensional fold (FIG. 22 left and right depictions). Leucine 104 is situated near the highly conserved MYPPPY amino acid sequence. Alanine 29 is situated near the C-terminal end of the CDR-1 (S25-R33) region, which is spatially adjacent to the MYPPPY region. While there is significant interaction between residues at the base of these two regions, there is apparently no direct interaction between L104 and A29 although they both comprise part of a contiguous hydrophobic core in the protein. The structural consequences of the two avidity enhancing mutants were assessed by modeling. The A29Y mutation can be easily accommodated in the cleft between the CDR-1 (S25-R33) region and the MYPPPY region, and may serve to stabilize the conformation of the MYPPPY region. In wild type CTLA4, L104 forms extensive hydrophobic interactions with L96 and V94 near the MYPPPY region. It is highly unlikely that the glutamic acid mutation adopts a conformation similar to that of L104 for two reasons. First, there is insufficient space to accommodate the longer glutamic acid side chain in the structure without significant perturbation to the CDR-1 (S25-R33 region). Second, the energetic costs of burying the negative charge of the glutamic acid side chain in the hydrophobic region would be large. Instead, modeling studies predict that the glutamic acid side chain flips out on to the surface where its charge can be stabilized by solvation. Such a conformational change can easily be accommodated by G105, with minimal distortion to other residues in the regions.

Binding of High Avidity Mutants to CHO Cells Expressing CD80 or CD86

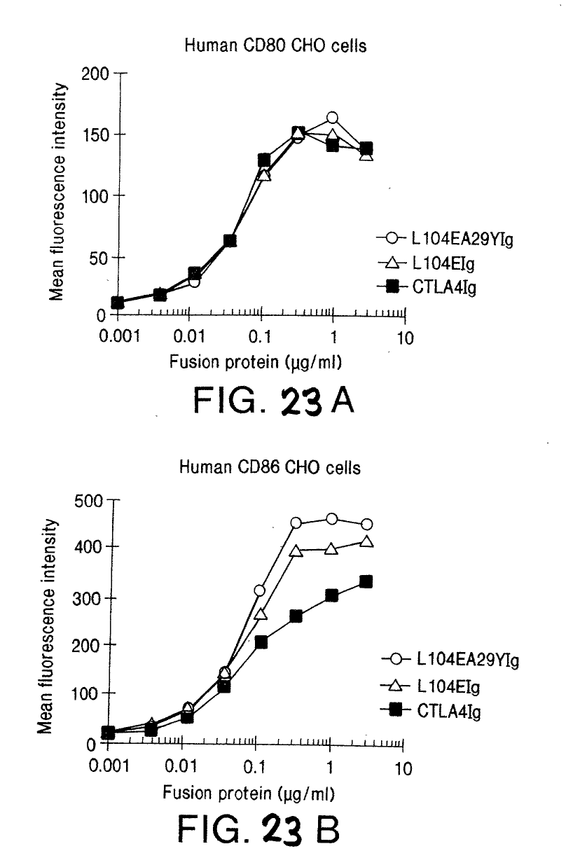

FACS analysis (FIG. 23) of CTLA4Ig and mutant molecules binding to stably transfected CD80+ and CD86+CHO cells was performed as described herein. CD80-positive and CD86-positive CHO cells were incubated with increasing concentrations of CTLA4Ig, L104EA29YIg, or L104EIg, and then washed. Bound immunoglobulin fusion protein was detected using fluorescein isothiocyanate-conjugated goat anti-human immunoglobulin.

As shown in FIG. 23, CD80-positive or CD86-positive CHO cells (1.5×10⁵) were incubated with the indicated concentrations of CTLA4Ig (closed squares), L104EA29YIg (circles), or L104EIg (triangles) for 2 hr. at 23° C., washed, and incubated with fluorescein isothiocyanate-conjugated goat anti-human immunoglobulin antibody. Binding on a total of 5,000 viable cells was analyzed (single determination) on a FACScan, and mean fluorescence intensity (MFI) was determined from data histograms using PC-LYSYS. Data were corrected for background fluorescence measured on cells incubated with second step reagent only (MFI=7). Control L6 mAb (80 μg/ml) gave MFI<30. These results are representative of four independent experiments.

Binding of L104EA29YIg, L104EIg, and CTLA4Ig to human CD80-transfected CHO cells is approximately equivalent (FIG. 23A). L104EA29YIg and L104EIg bind more strongly to CHO cells stably transfected with human CD86 than does CTLA4Ig (FIG. 23B).

Functional Assays

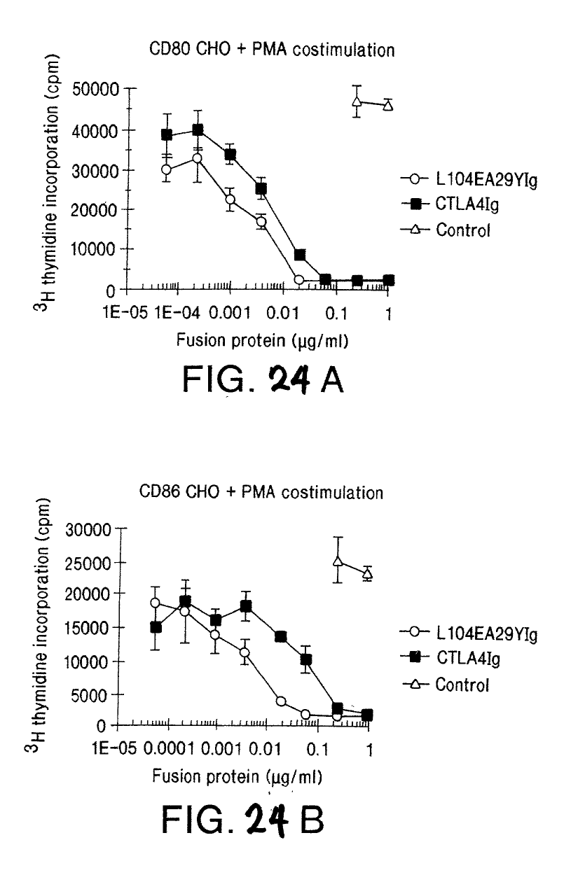

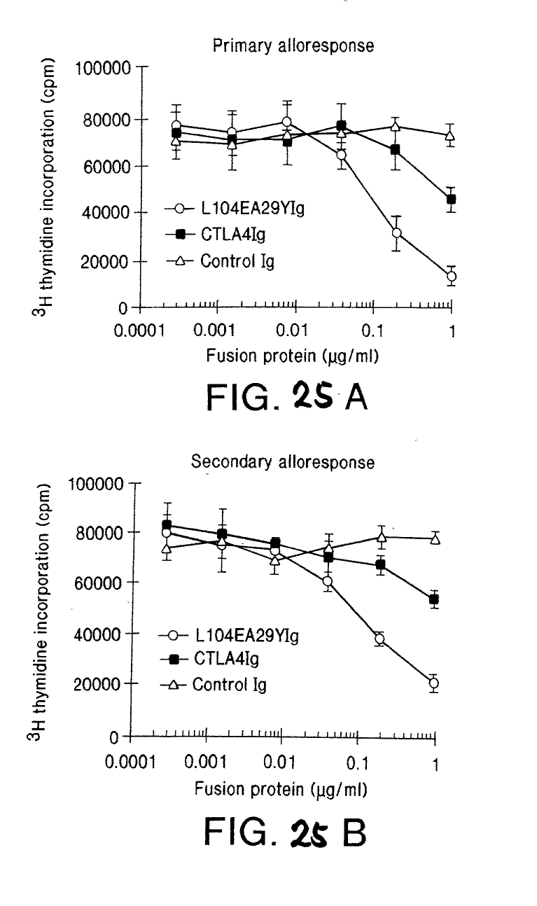

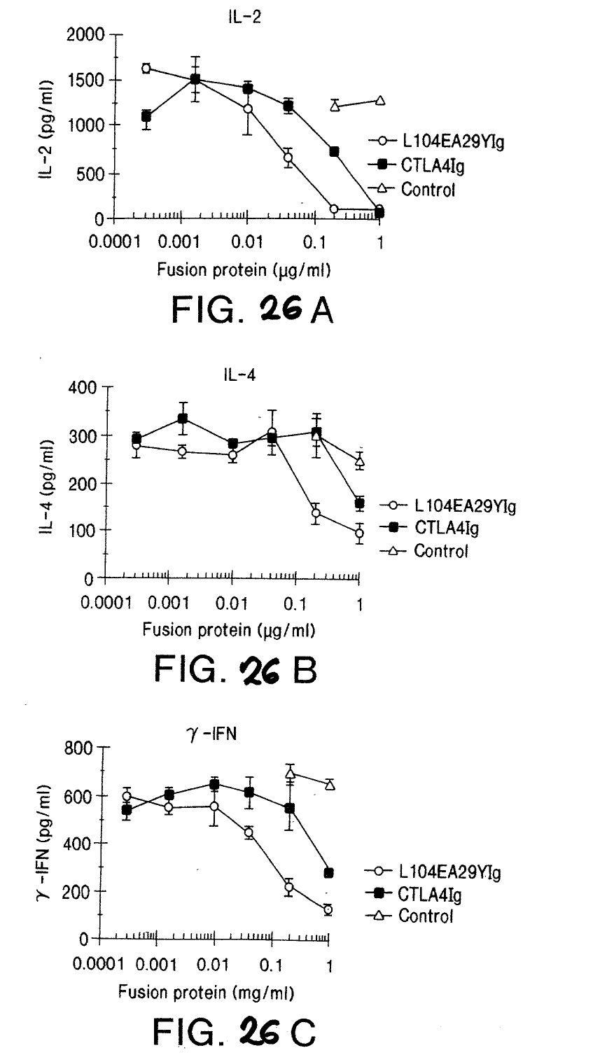

Human CD4-positive T cells were isolated by immunomagnetic negative selection (Linsley et al., (1992) J. Ex . Med. 176:1595-1604). Isolated CD4-positive T cells were stimulated with phorbal myristate acetate (PMA) plus CD80-positive or CD86-positive CHO cells in the presence of titrating concentrations of inhibitor. CD4-positive T cells (8-10×10⁴/well) were cultured in the presence of 1 nM PMA with or without irradiated CHO cell stimulators. Proliferative responses were measured by the addition of 1 μCi/well of [3H]thymidine during the final 7 hours of a 72 hour culture. Inhibition of PMA plus CD80-positive CHO, or CD86-positive CHO, stimulated T cells by L104EA29YIg and CTLA4Ig was performed. The results are shown in FIG. 24. L104EA29YIg inhibits proliferation of CD80-positive PMA treated CHO cells more than CTLA4Ig (FIG. 24A). L104EA29YIg is also more effective than CTLA4Ig at inhibiting proliferation of CD86-positive PMA treated CHO cells (FIG. 24B). Therefore, L104EA29YIg is a more potent inhibitor of both CD80- and CD86-mediated costimulation of T cells.

FIG. 25 shows inhibition by L104EA29YIg and CTLA4Ig of allostimulated human T cells prepared above, and further allostimulated with a human B lymphoblastoid cell line (LCL) called PM that expressed CD80 and CD86 (T cells at 3.0×10⁴/well and PM at 8.0×10³/well). Primary allostimulation occurred for 6 days, then the cells were pulsed with ³H-thymidine for 7 hours, before incorporation of radiolabel was determined.

Secondary allostimulation was performed as follows. Seven day primary allostimulated T cells were harvested over lymphocyte separation medium (LSM) (ICN, Aurora, OH) and rested for 24 hours. T cells were then restimulated (secondary), in the presence of titrating amounts of CTLA4Ig or L104EA29YIg, by adding PM in the same ratio as above. Stimulation occurred for 3 days, then the cells were pulsed with radiolabel and harvested as above. The effect of L104EA29YIg on primary allostimulated T cells is shown in FIG. 25A. The effect of L104EA29YIg on secondary allostimulated T cells is shown in FIG. 25B. L104EA29YIg inhibits both primary and secondary T cell proliferative responses better than CTLA4Ig.

To measure cytokine production (FIG. 26), duplicate secondary allostimulation plates were set up. After 3 days, culture media was assayed using ELISA kits (Biosource, Camarillo, Calif.) using conditions recommended by the manufacturer. L104EA29YIg was found to be more potent than CTLA4Ig at blocking T cell IL-2, IL-4, and γ-IFN cytokine production following a secondary allogeneic stimulus (FIGS. 26A-C).

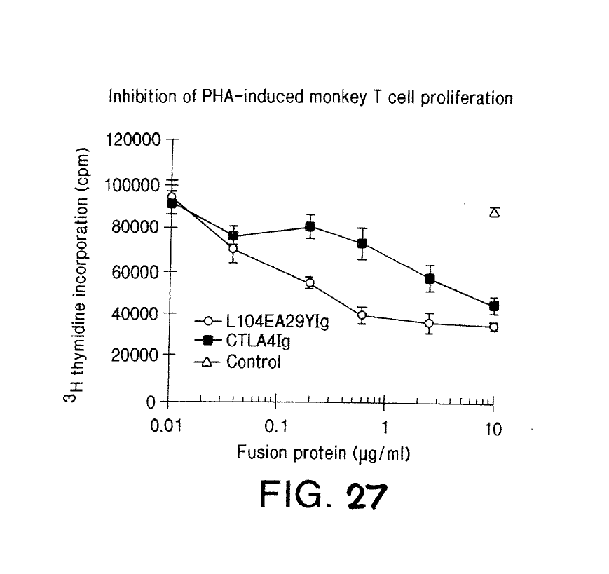

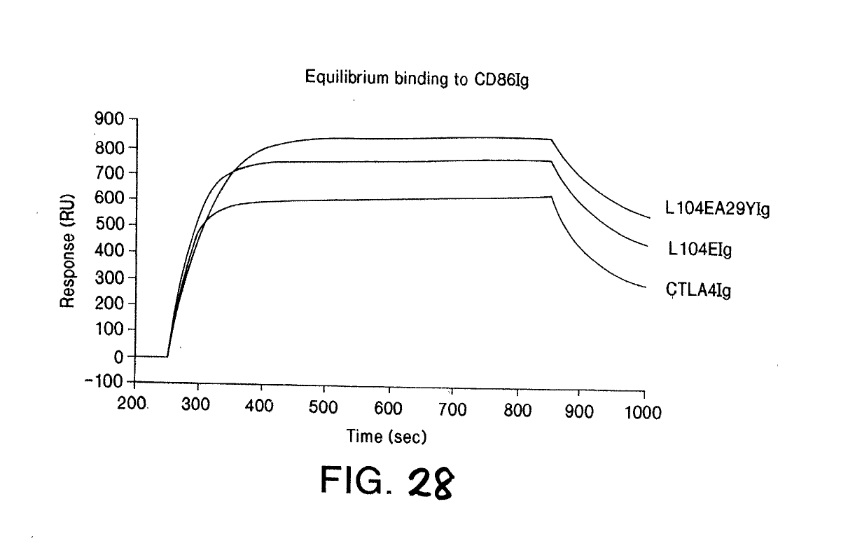

The effects of L104EA29YIg and CTLA4Ig on monkey mixed lymphocyte response (MLR) are shown in FIG. 27. Peripheral blood mononuclear cells (PBMC'S; 3.5×10⁴ cells/well from each monkey) from 2 monkeys were purified over lymphocyte separation medium (LSM) and mixed with 2 μg/ml phytohemaglutinin (PHA). The cells were stimulated 3 days then pulsed with radiolabel 16 hours before harvesting. L104EA29YIg inhibited monkey T cell proliferation better than CTLA4Ig.

Table I:

Equilibrium and apparent kinetic constants are given in the following table (values are means±standard deviation from three different experiments):

| Immo- | | | | |
|---|---|---|---|---|
| bilized | | kₒₙ (× 10⁵) | k_{off} (× 10⁻³) | K_{d} |
| Protein | Analyte | M⁻¹ S⁻¹ | S⁻¹ | nM |
| CD80Ig | CTLA4Ig | 3.44 ± 0.29 | 2.21 ± 0.18 | 6.51 ± 1.08 |
| CD80Ig | L104EIg | 3.02 ± 0.05 | 1.35 ± 0.08 | 4.47 ± 0.36 |
| CD80Ig | L104EA29YIg | 2.96 ± 0.20 | 1.08 ± 0.05 | 3.66 ± 0.41 |
| CD80Ig | CTLA4X_{C120S} | 12.0 ± 1.0 | 230 ± 10 | 195 ± 25 |
| CD80Ig | L104EA29YX_{C120S} | 8.3 ± 0.26 | 71 ± 5 | 85.0 ± 2.5 |
| CD86Ig | CTLA4Ig | 5.95 ± 0.57 | 8.16 ± 0.52 | 13.9 ± 2.27 |
| CD86Ig | L104EIg | 7.03 ± 0.22 | 4.26 ± 0.11 | 6.06 ± 0.05 |
| CD86Ig | L104EA29YIg | 6.42 ± 0.40 | 2.06 ± 0.03 | 3.21 ± 0.23 |
| CD86Ig | CTLA4X_{C120S} | 16.5 ± 0.5 | 840 ± 55 | 511 ± 17 |
| CD86Ig | L104EA29YX_{C120S} | 11.4 ± 1.6 | 300 ± 10 | 267 ± 29 |

Table II

The effect on CD86Ig binding by mutagenesis of CTLA4Ig at the sites listed was determined by SPR, described supra. The predominant effect is indicated with a “+” sign.

| | Effects of Mutagenesis | | |
|---|---|---|---|
| Mutagenesis | No Apparent | Slow “on” rate/ | Reduced ligand |
| Site | Effect | slow “off rate | binding |
| S25 | | + | |
| P26 | + | | |
| G27 | + | | |
| K28 | + | | |
| A29 | | + | |
| T30 | | + | |
| E31 | | | + |
| R33 | | | + |
| K93 | | + | |
| L96 | | + | |
| M97 | | | + |
| Y98 | | | + |
| P99 | | | + |
| P100 | | | + |
| P101 | | | + |
| Y102 | | | + |
| Y103 | | + | |
| L104 | | + | |
| G105 | | + | |
| I106 | + | | |
| G107 | + | | |
| Q111 | + | | |
| Y113 | + | | |
| I115 | + | | |

As will be apparent to those skilled in the art to which the invention pertains, the present invention may be embodied in forms other than those specifically disclosed above without departing from the spirit or essential characteristics of the invention. The particular embodiments of the invention described above, are, therefore, to be considered as illustrative and not restrictive.

### Example 10

The following example provides characterization of virus-mediated inhibition of mixed chimerism and allospecific tolerance. In particular, this example shows that LCMV infection impedes prolonged allograft survival following CD28/CD40 combined blockade.

Mice and Virus Infections.

Adult male 6- to 8-wk old BALB/c, B6, and C3H/HeJ mice were purchased from The Jackson Laboratory (Bar Harbor, ME). Mice were infected with 2×10⁵ PFU LCMV Armstrong injected intra-peritonealy (i.p.) Virus stocks were grown and quantitated as previously described (Ahmed et al., J. Exp. Med., 160:521 (1984)).

Skin Grafting.

Full thickness skin grafts (˜1 cm²) were transplanted on the dorsal thorax of recipient mice and secured with a Band-Aid (Johnson & Johnson, Arlington, Tex.) for 7 days. Graft survival was then followed by daily visual inspection. Rejection was defined as the complete loss of viable epidermal graft tissue. Statistical analyses were performed using a Mann-Whitney U test.

Bone Marrow Preparation and Treatment Protocols.

Skin graft recipients were treated with 500 μg each of hamster anti-murine CD40L Ab (MR1) and human CTLA4-Ig administered i.p. on the day of transplantation (day 0) and on postoperative days 2, 4, and 6. CD4- and CD8-depleted experimental groups received 100 μg of rat anti-mouse CD4 (GK1.5) or rat anti-mouse CD8 (TIB105) i.p. on days −3, −2, −1, and weekly until harvest. Mice treated with busulfan (Busulfex; Orphan Medical, Minnetonka, Minn.) received 600 μg on postoperative day 5. Bone marrow was flushed from tibiae, femurs, and humeri, and red blood cells were lysed using a Tris-buffered ammonium chloride solution. Cells were resuspended in saline and injected intra-venously (i.v.) at 2×10⁷ cells/dose on postoperative days 0 and 6.

CFSE Labeling and Adoptive Transfers.

Labeling of naive or immune B6 T cells and adoptive transfer into irradiated BALB/c recipients were performed as previously described (Williams et al., J. Immunol., 165:6849 (2000)). Harvested splenocytes were analyzed by flow cytometry.

Intracellular IFN-γ Assay.

Intracellular IFN-γ expression in response to restimulation with LCMV peptides was analyzed essentially as described (Murali-Krishna et al., Immunity 8:177 (1998)). In the case of irradiated recipients of CFSE-labeled cells, harvested splenocytes were incubated for 5 h with LCMV-infected or uninfected MC57 fibroblasts in the presence of brefeldin A (GolgiPlug; BD PharMingen, San Diego, Calif.). In the GVHD assay, peptide-specific IFN-γ production was assessed by restimulating with uninfected IC21 macrophage cells pulsed with the appropriate LCMV peptide at 0.1 μg/ml. After surface staining, cells were permeabilized and stained for IFN-γ expression using the Cytofix/Cytoperm kit (BD PharMingen) according to the manufacturer's instructions.

IFN-γ ELISPOT Assays.

Allospecific T cell responses were measured by IFN-γ ELISPOT assay. Three-fold dilutions of recipient splenocytes (H-2^{k} or H-2^{b}) were stimulated overnight with 5×10⁵ irradiated donor splenocytes (H-2^{d}) per well in ester-cellulose-bottom plates (Millipore, Bedford, Mass.) that had been previously coated with IFN-γ capture Ab. To measure LCMV-specific responses, splenocytes were restimulated overnight with infected L929 (H-2^{k}) or MC57 (H-2^{b}) cells. Plates were coated and developed as previously described (Williams et al., J. Immunol. 165:6849 (2000)).

Cell Preparations and Flow Cytometry.

MHC class I tetramers were prepared and refolded with β₂-microglobulin and the appropriate peptide as described previously (Murali-Krishna et al., Immunity 8:177 (1998)). Analyses of splenocytes of irradiated recipients of CFSE-labeled T cells were conducted using fluorochrome-conjugated Abs (rat IgG2a PE, rat IgG2b PE, anti-CD4 PE, anti-CD8 PE; BD PharMingen) and APC-labeled tetramers. For intracellular staining, cells were labeled with anti-CD8 PE and rat IgG2b APC or anti-IFN-γ APC (BD PharMingen). Peripheral blood was analyzed by staining with fluorochrome-conjugated Abs (rat IgG2a APC, anti-CD4 APC, mouse IgG2a FITC, anti-H-2K^{d}FITC, mouse IgG1 FITC, anti-Vβ5 FITC, rat IgG2b FITC, anti-Vβ11 FITC; BD PharMingen), followed by red blood cell lysis and washing with a whole-blood lysis kit (R&D Systems, Minneapolis, Minn.). Splenic dendritic cells were enriched on an Optiprep column (Nycomed, Oslo, Norway) as previously described (Ruedl et al., Eur. J. Immunol. 26:1801(1996)) and analyzed using fluorochrome-conjugated Abs (ham IgG PE, anti-CD11c PE, ham IgM FITC, anti-CD40 FITC, anti-CD54 FITC, rat IgG2a FITC, anti-CD80 FITC, anti-CD86 FITC, mouse IgG2a FITC, anti H-2K^{d} FITC, anti-1-A^{b} FITC; BD PharMingen). Flow cytometry was performed using a FACSCaliber, with CFSE fluorescence data being collected on the FL1 (FITC) channel. Data were analyzed using CellQuest software (BD Biosciences, Braintree, Mass.).

Cell Lines.

The fibrosarcoma cell line MC57 (H-2^{b+}) and the liver-derived cell line L929 (H-2^{k+}) were grown and passaged in RPMI 1640 supplemented with 10% FBS, antibiotics, and 2-ME.

Results

Acute LCMV Infection Disrupts Prolongation of Allograft Survival Induced by Blockade of the CD28/CD40 T Cell Costimulatory Pathways.

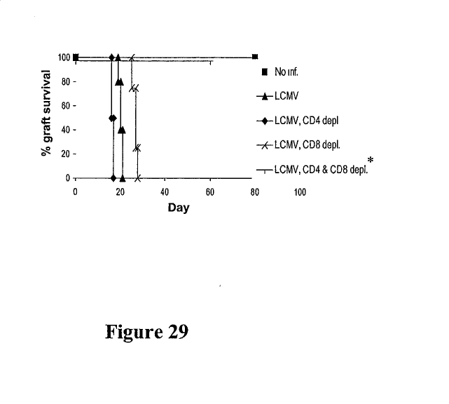

Recent evidence has indicated that some viral infections (e.g., LCMV and PV) inhibit the prolongation of skin allograft survival mediated by blockade of the CD40 pathway and administration of donor splenocytes (Welsh et al., J. Virol. 74:2210 (2000)). Whether acute LCMV infection could alter skin graft survival time when the CD28 and CD40 T cell costimulatory pathways were blocked simultaneously was assessed. C3H/HeJ mice received Balb/c skin allografts along with CTLA4-Ig and anti-CD40L on days 0, 2, 4, and 6 post transplant. The median survival time (MST) was >80 days (n=5). C3H/HeJ mice receiving BALB/c skin allografts and costimulation blockade survived >80 days (FIG. 29). In contrast, mice receiving the same procedure and treatment, along with a concomitant infection of 2×10⁵ LCMV Armstrong on or near the day of transplant, rejected their grafts promptly (MST=20 days; FIG. 29). To determine the relative contributions of each T cell subset to the costimulation blockade-resistant rejection of skin allografts following acute LCMV infection, CD4⁺ and CD8⁺ T cells were depleted in vivo with GK1.5 and TIB105 Abs, respectively. As seen in FIG. 29, depletion of CD4⁺ T cells did not alter the ability of C3H/HeJ mice to reject BALB/c skin grafts following infection with LCMV (MST=18 days). Depletion of CD8⁺ T cells resulted in a slight delay of skin graft rejection (MST=26 days). Depletion of both subsets simultaneously resulted in long-term allograft survival (MST >60 days) (FIG. 29), indicating both that the depletions were effective and that the virus was not directly harmful to the allograft.

These results demonstrate that LCMV induces accelerated graft rejection in the face of combined blockade of the CD28 and CD40 T cell costimulatory pathways. The results also indicate that either CD4⁺ or CD8⁺ T cells are sufficient to mediate LCMV-induced skin graft rejection.

Acute LCMV Infection Impedes the Establishment of Partial Hematopoietic Chimerism, Deletion of Alloreactive T Cells, and the Induction of Donor-Specific Tolerance.

To determine whether LCMV infection had the same effect in a robust tolerance induction model, Specifically, whether acute LCMV infection could disrupt the costimulation blockade-mediated establishment of mixed hematopoietic chimerism and donor-specific tolerance. Administration of donor bone marrow following treatment with the selective stem cell toxin busulfan, together with blockade of the CD40/CD28 costimulatory pathways, results in high levels of chimerism, deletion of donor-reactive T cells, and indefinite donor-specific tolerance (Adams et al., J. Immunol. 167:1103 (2001)).

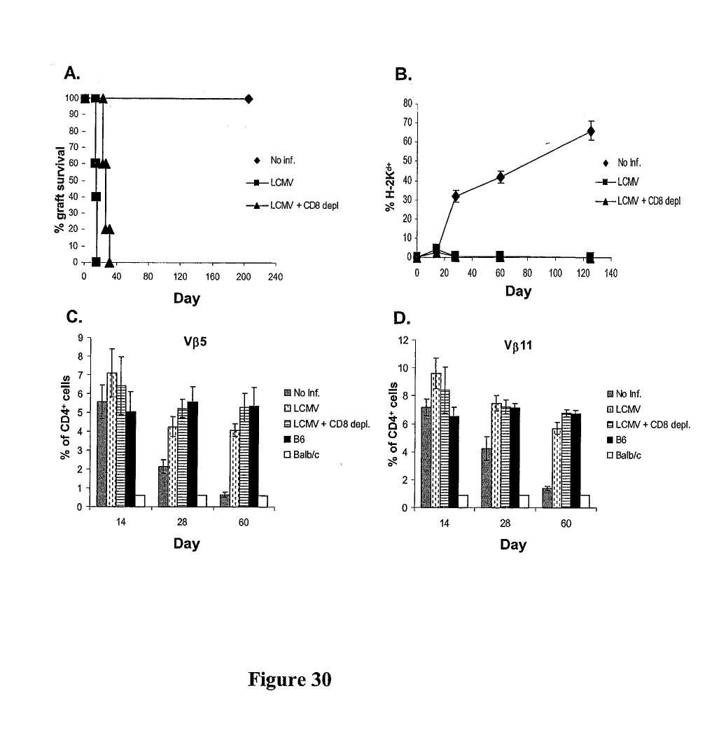

As seen in FIG. 30A, 5/5 B6 mice receiving BALB/c skin and bone marrow, as well as busulfan and costimulatory blockade treatment, had greater than 200-day skin graft survival in 100% of mice tested. Conversely, 5/5 mice receiving the same treatment concomitantly with an acute LCMV infection rejected their grafts promptly (MST=14 days). These results are representative of three separate experiments. As in the previous model, predepletion of CD8⁺ T cells demonstrated that CD4⁺ T cells can mediate graft rejection, although in a somewhat delayed fashion. Following depletion, no CD8⁺ T cells could be detected in the peripheral blood, while simultaneous depletion of both subsets during infection resulted in indefinite graft survival, indicating that the depletions were effective.

Following the aforementioned procedure, uninfected mice proceeded to develop substantial levels of hematopoietic chimerism (FIG. 30B). By day 125, greater than 60% of peripheral blood leukocytes were H-2K^{d+} in all the mice (n=5). Chimerism was seen in all lineages tested, including CD4⁺, CD8⁺, B220⁺, CD11b⁺, and GR-1⁺ cells. Conversely, mice receiving the same treatment along with LCMV at the time of engraftment never developed detectable long-term chimerism. Predepletion of CD8⁺ T cells (FIG. 30B) did not alter the ability of the infection to abrogate chimerism.

Donor-specific tolerance following bone marrow engraftment and treatment with costimulation blockade is due at least in part to deletion of alloreactive T cells (Wekerle et al., J. Exp. Med. 187:2037 (1998), Durham et al., J. Immunol. 165:1 (2000)). To determine whether LCMV-induced skin graft rejection was associated with impaired peripheral deletion of donor-reactive T cells, the use of Vβ11 and Vβ5.1/2 by CD4⁺ T cells from B6 recipients in the uninfected group (accepted both bone marrow and skin grafts) and from the infected groups (rejected bone marrow and skin grafts) was compared. BALB/c mice delete Vβ11 and Vβ5-bearing T cells in the thymus due to their high affinity for endogenous retroviral superantigens (mouse mammary tumor virus (MMTV)) presented by I-E MHC class II molecules. B6 mice do not express I-E and thus use Vβ11 on ˜5-7% of CD4⁺ T cells and Vβ5.1/2 on ˜3-5% of CD4⁺ T cells. In this experiment, uninfected mice treated with costimulation blockade, bone marrow, and busulfan following skin engraftment showed decreased percentages of Vβ11⁺CD4⁺ and Vβ5⁺CD4⁺ T cells in the peripheral blood by day 28 post-transplant. At day 60 posttransplant, expression of these cell populations was nearly undetectable in the peripheral blood, comprising similar percentages of the total CD4⁺ population as those found in BALB/c mice. In contrast, mice receiving 2×10⁵ PFU LCMV Armstrong at the time of engraftment failed to delete Vβ5⁺CD4⁺ and Vβ11⁺CD4⁺ T cells at any time posttransplant (FIG. 30, C and D). Failure to delete these cell populations occurred regardless of the presence of CD8⁺ T cells. This correlates with earlier observations noting an LCMV-induced inhibition of peripheral deletion of alloreactive T cells following disruption of the CD40/CD40L pathway (Turgeon et al., J. Surg. Res. 93:63 (2000)).

These results indicate a role for LCMV in overcoming the tolerizing effects of combined costimulation blockade and bone marrow administration. The data shows that there is rapid rejection of skin and hematopoietic allografts following acute infection, preventing the induction of donor-specific tolerance. Additionally, heterotopic heart allografts have been performed using the same treatment, and again LCMV inhibits the generation of donor-specific tolerance. This effect cannot be attributed to either CD8⁺ or CD4⁺ T cells alone, as either subpopulation appears capable of inducing rapid CD40/CD28-independent graft rejection following acute infection. As predicted by graft survival, donor-reactive T cells are not deleted in infected mice, whereas uninfected mice receiving the tolerizing regimen delete donor MMTV superantigen-reactive T cell subpopulations within 60 days.

LCMV Infection Does Not Abrogate Established Tolerance.

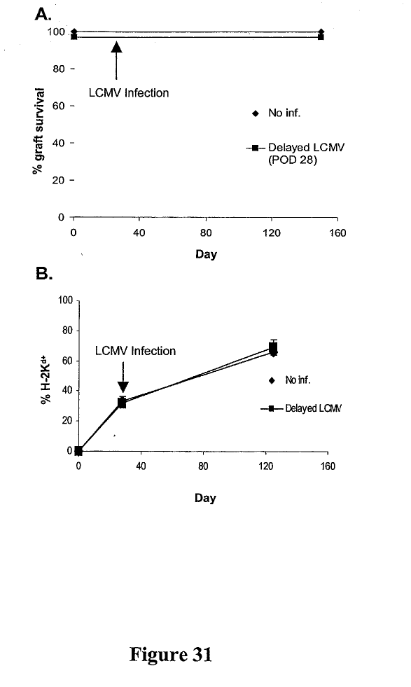

It is unlikely that a delayed infection with LCMV could induce rejection of skin or bone marrow grafts in tolerant chimeric mice. To test this hypothesis, mice were infected with LCMV 4-5 wk following transplantation and tolerance induction. 5/5 mice were greater than 20% chimeric in the peripheral blood at the time of infection. Following infection, skin graft survival and the development of chimerism were monitored. As seen in FIG. 31, A and B, skin grafts on mice receiving a delayed LCMV infection survived indefinitely, while hematopoietic chimerism developed normally, as compared with uninfected controls.

LCMV infection may generate a T cell response that is cross-reactive with the alloantigen at the level of the TCR, and this response is essential for LCMV-induced graft rejection. Given previous results showing the deletion of donor-reactive T cells and an inability to detect their presence in chimeric tolerant mice (Wekerle et al., J. Exp. Med., 187:2037 (1998); Durham et al., J. Immunol., 165:1 (2000)), this would predict that alloreactive T cells that also recognize an LCMV epitope would be deleted or inactivated by day 28. Therefore, one might expect an alteration of the repertoire of T cells available to respond to LCMV in the tolerant mice. To test this possibility, LCMV-specific immune responses were analyzed in tolerant mice. Selective impairment of the T cell response to any particular epitope was consistent with TCR cross-reactivity.

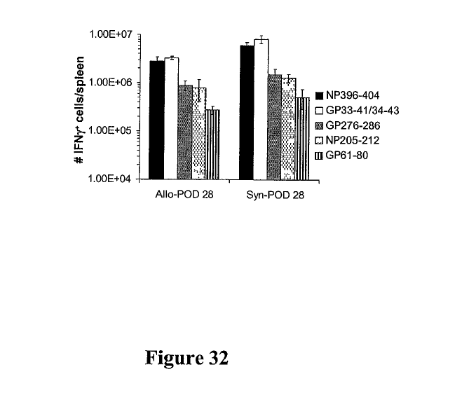

B6 mice received BALB/c skin grafts and bone marrow, along with costimulatory blockade and busulfan treatment. Control mice received the same treatment regimen following receipt of syngeneic bone marrow and skin grafts. On day 28 post-transplant, mice were infected with LCMV. Eight days later splenocytes were harvested, restimulated for 5 h with LCMV peptides in the presence of brefeldin A, and stained for intracellular IFN-γ expression. The peptides tested were nucleoprotein (NP)₃₋₉₆-404, gp33-41, gp276-286, NP205-214, and the class II-restricted peptide gp61-80. All epitopes tested generated large numbers of Ag-specific T cells in the spleen by day 8 postinfection in animals receiving syngeneic skin and bone marrow grafts. In mice receiving allogeneic grafts, the number of antiviral T cells in the spleen 8 days postinfection was moderately lower for each epitope tested (1.5- to 2-fold), possibly due to the influx of APCs not expressing H-2^{b}. However, no substantial deletion of any particular epitope could be detected, nor was there any apparent change in epitope hierarchy between the mice receiving syngeneic grafts and the mice receiving allogeneic grafts (see FIG. 32). These results suggest that there is a moderate reduction of antiviral responses following the induction of mixed chimerism, but do not implicate TCR cross-reactivity of any specific epitope to alloantigen.

LCMV-Specific T Cells Fail to Divide in Response to Alloantigen.

To directly address the question of whether LCMV-specific CD8 T cells were also alloreactive, a previously described GVHD model (Wells et al., J. Clin. Invest. 100:3173 (1997)) was used. T cells from LCMV-immune B6 mice (>30 days postinfection) were labeled with the fluorescent dye CFSE (Molecular Probes, Eugene, Oreg.) and injected i.v. into irradiated (1800 rad) allogeneic BALB/c hosts. In this model, allogeneic T cells responding to Ag lose fluorescence with each successive division, allowing for quantitation and analysis of highly divided alloreactive cells by flow cytometry. By using LCMV-immune mice as donors, we assessed whether LCMV-reactive T cells also divided in response to alloantigen by direct staining with the D^{b}/NP396-404, D^{b}/gp33-41, and K^{b}/gp34-43 class I MHC tetramers. Splenocytes were harvested 72 h posttransfer, stained with anti-CD8 Abs and tetramers, and analyzed by flow cytometry.

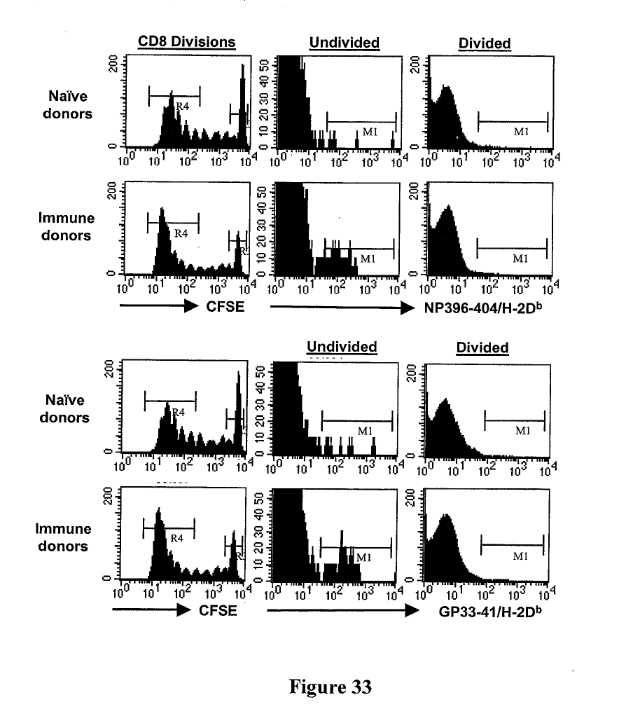

CD8⁺ T cells from both naive and immune mice divided significantly in response to alloantigen, with large numbers of cells from both groups reaching at least eight divisions. In contrast, CD8⁺ T cells from either group injected into irradiated syngeneic recipients did not divide more than three times. Therefore, gated undivided and maximally divided (four to eight divisions) CD8⁺ T cells were gated and analyzed tetramer binding in each population (FIG. 33). LCMV-specific CD8 T cells were readily detectable within the undivided population in the recipients of LCMV-immune T cells for each tetramer tested. However, discernible staining was not detected above background for any of the tetramers in the maximally divided population (FIG. 33). The results are summarized in Table Ill. As a control to verify that failure to detect tetramer binding was not simply a result of TCR down-modulation in highly divided cells, expression of TCRβ was determined by staining. Decreased expression of the TCR in the maximally divided cells was not observed by this assay. Furthermore, a previous study has established that proliferating LCMV-specific CD8 T cells in lymphopenic hosts do not show decreased binding to MHC tetramers (Murali-Krishna et al., J. Immunol., 165:1733 (2000)).

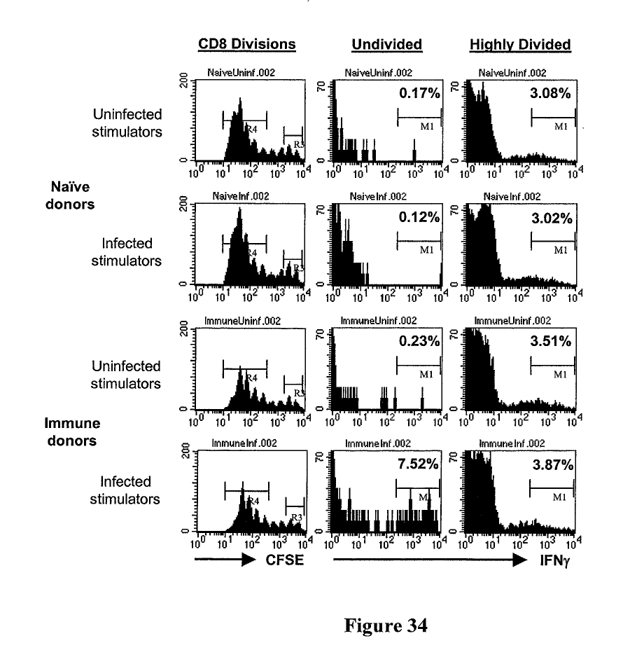

Although this experiment excludes the three principal epitopes as candidates for alloreactivity, whether cross-reactivity could be detected in donor cells from immune mice was determined following restimulation with whole LCMV and specific LCMV peptides in vitro. To achieve this, mouse splenocytes were harvested from BALB/c recipients on day 3 as above, restimulated for 5 h with infected or uninfected MC57 cells and brefeldin A, permeabilized and stained for intracellular IFN-γ expression, and analyzed by flow cytometry. As seen in FIG. 34, no IFN-γ expression was observed above background except in the undivided CD8 T cells stimulated with infected MC57 cells. Responses to four LCMV epitopes were further analyzed in the same manner. In these experiments, rather than stimulating with infected cells, harvested splenocytes were restimulated with MC57 cells pulsed with LCMV peptides (NP396-404, gp3341, gp276-286, NP205-214). None of these peptides induced IFN-γ production above background in the divided, alloreactive CD8 T cells. In contrast, LCMV-specific CD8 T cells could be readily detected in the undivided population following restimulation with these peptides. One caveat to these experiments is the high background IFN-γ production in the highly divided cells, presumably due to the continued cycling and low-level stimulation of these cells during brefeldin A incubation.

TABLE III
LCMV-specific T cells fail to divide in response to alloantigen.

LCMV Immune Donors

| Tetramer | 0-1 div. | 4-8 div. |
|---|---|---|
| D^{b}/NP396-404 | 1.83 ± .046% | .13 ± .074% |
| D^{b}/GP33-41 | 1.59 ± .088% | .15 ± .135% |
| K^{b}/GP34-43 | 1.73 ± .250% | .26 ± .094% |
| 2 × 10⁷ CFSE labeled T cells from LCMV immune (>30 days post-infection) B6 mice were injected i.v. into irradiated (1800 rads) Balb/c mice. Recipient splenocytes were harvested 72 hours later and analyzed by three color flow cytometry for expression of CD8, CFSE fluorescence, and MHC tetramer binding. Numbers indicate the percent of CD8+ T cells in the indicated divided | | |
| # populations that bound MHC tetramer. The indicated error represents the standard error of the mean (n = 3). | | |

LCMV Facilitates the CD28/CD40-Independent Generation of Alloreactive IFN-γ-Producing Cells.

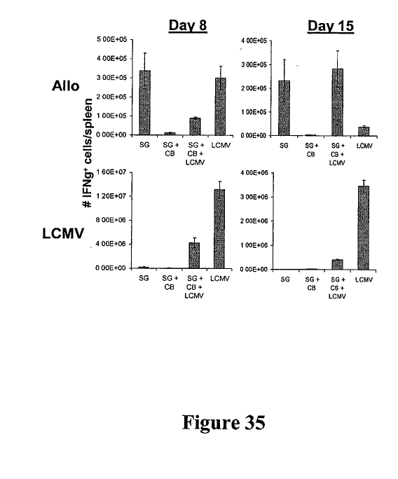

To better characterize the generation of allogeneic and antiviral T cell responses following LCMV infection, splenocytes were monitored for their ability to produce IFN-γ after restimulation in vitro by an ELISPOT assay. In this experiment, C3H/HeJ mice receiving BALB/c skin grafts generated ˜3-4×10⁵ allospecific T cells in the spleen by day 8 post-transplant, and these cell numbers dropped slightly at day 15. Treatment with costimulation blockade completely abolished the allogeneic response at both time points. Mice receiving skin grafts and costimulation blockade concurrent with an acute LCMV infection generated small numbers (˜9×10⁴) of allospecific cells in the spleen by day 8. By day 15, these mice had overcome the immunosuppressive effects of costimulation blockade and had generated an alloresponse comparable to untreated controls (˜2.5×10⁵). As reported previously, acute LCMV infection in the absence of a skin graft resulted in the generation of some allospecific IFN-γ-producing cells by day 8 (˜3×10⁵). By day 15, this effect had diminished markedly to ˜4×10⁴ IFN-γ⁺ cells per spleen (FIG. 35).

To measure the LCMV-specific response, splenocytes from each group were incubated with infected L929 cells overnight on an ELISPOT plate. As expected, LCMV infection alone induced a potent antiviral response, generating ˜1.2×10⁷ IFN-γ-producing T cells in the spleen, whereas mice that received concurrent combination blockade and a BALB/c skin graft, while still generating a large response, had an ˜3-fold drop in the number of LCMV-specific cells in the spleen (˜4×10⁶). By day 15, spleens from LCMV-infected mice showed a 3- to 4-fold decrease in the number of LCMV-specific cells. In mice receiving combination blockade, the drop was somewhat greater (FIG. 35).

To assess whether LCMV-infected mice generated memory to alloantigen, B6 mice were infected and the number of allospecific cells in the spleen were quantitated at the peak of the infection (day 8) and following the development of immune memory (>30 days postinfection) by IFN-γ ELISPOT. LCMV-infected mice generated allospecific T cells (7.29×10⁵ ±1.78×10⁵, n=3) at the peak of the infection, but by day 30 postinfection, the number of these cells in the spleen dropped 50- to 100-fold (1×10⁴±9.9×10², n=3). In contrast, the number of T cells specific for several known immunodominant and subdominant LCMV epitopes (NP396-404, gp33-41, gp276-286, NP205-214) dropped 10- to 12-fold in the spleen over the same period (1.52×10⁷±1.13×10⁶ to 1.40×10⁶±1.38×10⁵, n=3 for both groups). This level of LCMV memory is similar to previous reports (Murali-Krishna et al., Immunity, 8:177 (1998)).

These results demonstrate that LCMV infection stimulates the activation of at least a subset of allogeneic T cells by CD40/CD28-independent mechanisms, thereby overcoming the immunosuppressive effects of costimulation blockade and leading to early graft rejection. Based on the CFSE and ELISPOT results, it is likely that the frequency of virus-specific T cells also bearing TCR specificity to alloantigen is low.

LCMV Infection Induces the CD28/CD40-Independent Maturation of Splenic Dendritic Cells.

Other potential mechanisms whereby LCMV infection could abrogate transplant tolerance and stimulate the activation of alloreactive T cells were explored. Previous experiments studying deletion of Vβ subsets established that in the presence of LCMV infection, CTLA4-Ig and anti-CD40L are unable to initiate the deletion of alloreactive T cells. LCMV infection maybe able to influence the induction and/or up-regulation of T cell costimulatory pathways by APCs. Furthermore, LCMV might induce the expression of molecules or survival factors that prevented deletion of alloreactive T cells. To test this, the effects of LCMV infection on costimulatory molecule and MHC expression by CD11c⁺ dendritic cells in the spleen was analyzed.

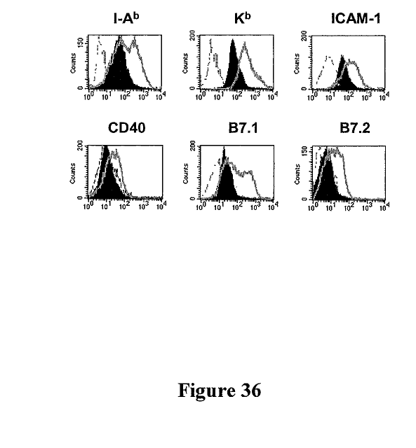

Mice received BALB/c skin grafts and bone marrow, costimulatory blockade therapy, and busulfan. One group was infected with LCMV Armstrong on day 0, while the other remained uninfected. Splenocytes were harvested on day 6 and separated based on cell density using an Optiprep column (Nycomed) as previously described (Ruedl et al., Eur. J. Immunol. 26:1801(1996)). The low-density fraction, which is enriched for dendritic cells, was harvested and stained for CD11c expression, along with MHC class I and II, ICAM-1, CD40, CD80, and CD86. Following analysis by flow cytometry, expression of these molecules among CD11c⁺ cells was analyzed. As seen in FIG. 36, LCMV infection resulted in the increased expression of all of these molecules, regardless of the presence of costimulatory blockade. Thus LCMV infection induces a higher activation state among dendritic cells. The deleterious effects of LCMV infection on tolerance induction may be due to the increased ability of APCs to stimulate and activate alloreactive T cells.

This Example shows that LCMV infection causes rapid allograft rejection following combined therapy with CTLA4-Ig and anti-CD40L. This effect can be extended to a robust tolerance induction model, as LCMV infection impedes both indefinite skin allograft survival as well as mixed hematopoietic chimerism following administration of donor bone marrow, busulfan, CTLA-Ig, and anti-CD40L. Although this effect is somewhat delayed in the absence of CD8 T cells, it nonetheless occurs without detectable CD8 expression in the blood, and depletion of CD4 T cells has little to no effect on graft survival. LCMV-induced allograft rejection correlates with a failure to delete donor-reactive CD4 T cells, as measured by tracking superantigen-reactive Vβ T cell subsets. Infection must occur at or around the time of transplant, as a delay of 3-4 wk in the onset of infection has no effect on graft survival or the induction of mixed chimerism. These studies confirm prior reports of the LCMV-mediated abrogation of skin graft survival following administration of donor splenocytes and anti-CD40L (Welsh et al., J. Virol., 74:2210 (2000)), and extend them by showing that LCMV-induced graft rejection is not mediated by CD40-independent up-regulation of B7.1 or B7.2. One concern with the use of tolerance induction strategies is the potential to induce tolerance to concurrent viral infections. It is of considerable interest that the immune responses to LCMV are not rendered tolerant following the use of costimulation blockade-based tolerance induction regimens, a finding consistent with previous observations that LCMV T cell responses are largely independent of CD28 and CD40 (Whitmire et al., J. Virol., 70:8375 (1996); Andreasen et al., J. Immunol., 164:3689 (2000); Shahinian et al., Science 261:609 (1993)).

It has been proposed that one possible explanation for the deleterious effects of LCMV infection on graft survival could be the presence of cross-reactivity to alloantigen at the level of TCR/MHC recognition during an antiviral response (Welsh et al., J. Virol., 74:2210 (2000)). In this scenario, antiviral responses would include some cells also bearing specificity for alloantigen. In support of this hypothesis, it has been shown that LCMV induces H-2^{d}-specific CD8 T cells at the peak of the T cell response (Yang et al., J. Immunol., 136:1186 (1986)). Although the above example provide similar results, there is little evidence for substantial cross-reactivity of LCMV-specific CD8 T cells generated in vivo to H-2^{d} alloantigen. A delayed primary infection (4 wk post-transplant) elicits an antiviral response with unchanged epitope hierarchy, although the numbers of activated CD8 T cells are somewhat globally diminished. Furthermore, using a sensitive single cell assay using intracellular cytokine staining and MHC tetramers, the division of LCMV-immune CD8 T cells in response to alloantigen was not detected. Nevertheless, as has been previously reported, LCMV primary infection does generate alloreactive cells. These cells drop greatly in number by day 15 post-infection and are barely detectable in LCMV-immune mice (>30 days post-infection). These experiments suggest that the frequency of LCMV-specific CD8 T cells that are cross-reactive to alloantigen is low and high levels of cross-reactive CD4 T cells may be present.

The primary mechanism by which alloreactive T cells are activated during LCMV infection remains unknown. Studies in recent years have shown that the great majority of activated CD8 T cells generated during an antiviral response are Ag-specific (Murali-Krishna et al., Immunity 8:177 (1998); Butz et al., Immunity 8:167 (1998); Zarozinski et al., J. Exp. Med. 185:1629 (1997)). Given the high frequency of alloreactive CD8 T cells in naive mice, there may be substantial cross-reactivity at the level of TCR/MHC interaction. However, we are unable to detect significant levels of allospecific activation of CFSE labeled LCMV specific CD8 T cells following injection into irradiated BALB/c donors. Furthermore, LCMV-induced alloreactive cells do not behave as other virus-specific populations, as they have an exaggerated death phase following the peak of the response. Both CD4 and CD8 T cell subsets in isolation are capable of preventing tolerance induction and mixed chimerism. Interestingly, disruption of costimulatory pathways during LCMV infection has little effect on CD8⁺ T cell responses but blocks the generation of CD4⁺ antiviral T cells (Whitmire et al., J. Immunol., 163:3194 (1999)). Nevertheless, CD4⁺ T cells are sufficient to mediate the LCMV-induced prevention of tolerance induction to alloantigen. Although cross-reactivity to alloantigens likely exists at some level, this data suggest that this is a relatively infrequent event during LCMV infection. It is of interest to note that LCMV responses are diminished in mice receiving the tolerance induction regimen. This observation could be due to nonspecific immunosuppressive effects of allogeneic bone marrow and costimulation blockade treatment. Alternatively, the influx of H-2^{d+} donor APCs in the immune compartments could dilute the available Ag for stimulating an H-2^{b}-restricted response. Further studies are warranted to assess the long-term effects of tolerance induction on immune responses to other pathogens.

Regardless of the extent to which alloreactive cells are generated during primary LCMV infection through TCR cross-reactivity, other mechanisms clearly play an indispensable role in the LCMV-mediated circumvention of the CD28/CD40 pathways. For example, MCMV and VV both generate allogeneic responses during primary infection (Yang et al., J. Immunol., 142:1710 (1989)), yet infection with these viruses has been shown not to impair graft survival (Welsh et al., J. Virol., 74:2210 (2000)). The primary CD8⁺ anti-LCMV response itself has been shown to be largely independent of the CD28 and CD40 pathways (Whitmire et al., J. Virol., 70:8375 (1996); Andreasen et al., J. Immunol., 164:3689 (2000); Shahinian et al., Science 261:609 (1993)). Interestingly, a recent study demonstrates that LCMV-specific responses, but not those directed toward VV, can be driven by parenchymal cells (Lenz et al., J. Exp. Med., 192:1135 (2000)). This suggests that LCMV, but not UV, can lower the threshold required for full activation of effector cells. One possibility is that LCMV triggers specific innate immune mechanisms that allow for the circumvention of these pathways in generating T cell responses. Also, anti-LCMV responses may provide cytokines and growth factors that aid the generation of CD28/CD40 independent alloresponses. Another possibility is that LCMV infection induces the expression of CD40/CD28-independent costimulatory pathways. In support of this latter possibility, the above example shows that LCMV infection mediates the CD28/CD40-independent up-regulation of MHC and costimulatory molecules on dendritic cells. Thus, infection with LCMV may facilitate the activation of alloreactive cells in the face of costimulatory blockade through the up-regulation of alternative costimulatory molecules on the surface of APCs. In this model, the need for costimulation and activation of dendritic cells by the CD28 or CD40 pathways would be abrogated by infection with LCMV.

As will be apparent to those skilled in the art to which the invention pertains, the present invention may be embodied in forms other than those specifically disclosed above without departing from the spirit or essential characteristics of the invention. The particular embodiments of the invention described above, are, therefore, to be considered as illustrative and not restrictive. The scope of the present invention is as set forth in the appended claims rather than being limited to the examples contained in the foregoing description.

What is claimed is:

1. A method of inhibiting rejection of a solid organ transplant in a subject having a transplanted tissue comprising:
a) administering an alkylating agent to the subject; and
b) subsequently administering T cell depleted bone marrow cells to the subject at approximately the same time as the solid organ transplant, thereby inhibiting rejection of the solid organ or tissue/cellular transplant.

2. The method of claim 1, wherein the alkylating agent is busulfan.

3. The method of claim 1 further comprising the step of administering to the subject an immunosuppressive composition that blocks T cell costimulatory signals in the subject.

4. The method of claim 3, wherein the immunosuppressive composition comprises a combination of a first ligand that interferes with binding of CD28 to either CD80 or CD86, and a second ligand that interferes with binding of CD154 to CD40.

5. The method of claim 4, wherein the first ligand is a soluble CTLA4 molecule.

6. The method of claim 4, wherein the first ligand is CTLA4-Ig.

7. The method of claim 4, wherein the second ligand is an anti-CD 154 mAb.

8. The method of claim 4, wherein the first ligand is a soluble CTLA4 molecule and the second ligand is an anti-CD154 mAb.

9. A method for establishing mixed hematopoietic chimerism in a subject having a transplanted tissue comprising:
a) administering T cell depleted bone marrow cells to the subject;
b) administering an alkylating agent to the subject; and
c) administering an immunosuppressive composition that blocks T cell costimulatory signals in the subject,
thereby establishing hematopoietic chimerism in the subject.

10. The method of claim 9, wherein the alkylating agent is busulfan.

11. The method of claim 9, wherein the immunosuppressive composition comprises a combination of a first ligand that interferes with binding of CD28 to either CD80 or CD86, and a second ligand that interferes with binding of CD 154 to CD40.

12. The method of claim 11, wherein the first ligand is a soluble CTLA4 molecule.

13. The method of claim 11, wherein the first ligand is CTLA4-Ig.

14. The method of claim 11, wherein the second ligand is an anti-CD154 mAb.

15. The method of claim 11, wherein the first ligand is a soluble CTLA4 molecule and the second ligand is an anti-CD154 mAb.

16. The method of claim 9, wherein the method inhibits rejection of an organ or tissue transplanted into the subject.

17. The method of claim 9, wherein the T cell depleted bone marrow is administered in at least two doses.

18. A method for establishing mixed hematopoietic chimerism in a subject having a transplanted tissue comprising:
a) administering T cell depleted bone marrow cells to the subject;
b) administering an immunosuppressive composition that blocks T cell costimulatory signals in the subject; and
c) administering busulfan to the subject,
thereby establishing mixed hematopoitic chimerism in the subject.

19. The method of claim 18, wherein the immunosuppressive composition comprises a combination of a first ligand that interferes with binding of CD28 to either CD80 or CD86, and a second ligand that interferes with binding of CD154 to CD40.

20. The method of claim 19, wherein the first ligand is a soluble CTLA4 molecule.

21. The method of claim 19, wherein the first ligand is CTLA4-Ig.

22. The method of claim 19, wherein the second ligand is an anti-CD154 mAb.

23. The method of claim 19, wherein the first ligand is a soluble CTLA4 molecule and the second ligand is an anti-CD154 mAb.

24. A method for treating hemoglobinopathy in a subject by establishing hematopoietic chimerism by the method of claim 9, or 18.

25. The method of claim 24, wherein hemoglobinopathy is beta-thalassemia.

26. The method of claim 24, wherein the hemoglobinopathy is sickle cell disease.

27. The method of claim 1, 9, or 18, wherein the transplanted tissue is a solid organ or tissue/cellular transplant.

28. The method of claim 9 or 18, wherein steps (b) and (c) are concurrent.

29. The method of claim 9 or 18, wherein steps (b) and (c) are subsequent to step (a).

30. The method of claim 2, 10 or 18, wherein the busulfan is admitnistered within one day prior to the solid organ or tissue/cellular transplant.

31. The method of claim 2, 10 or 18, wherein the busulfan is administered within twelve hours prior to the solid organ or tissue/cellular transplant.

32. The method of claim 2, 10 or 18, wherein the busulfan is administered within six hours prior to the solid organ or tissue/cellular transplant.

33. The method of claim 1, 9, or 18, wherein the transplanted tissue is a skin graft.

34. A method of reducing rejection of an organ transplant in a subject in need thereof comprising:
a) administering a first dose of T cell depleted bone marrow cells and an immunosuppressive composition to a subject;
b) placement of an organ or tissue/cellular transplant to the subject;
c) administering busulfan to the subject; and
d) administering a second dose of T cell depleted bone marrow cells and an immunosuppressive agent,
thereby reducing rejection of the organ or tissue/cellular transplant.

35. The method of claim 34, wherein the immunosuppressive agent is a combination of a first ligand that interferes with binding of CD28 to either CD80 or CD86, and a second ligand that interferes with binding of CD154 to CD40.

36. The method of claim 35, wherein the first ligand is a soluble CTLA4 molecule.

37. The method of claim 35, wherein the first ligand is CTLA4-Ig.

38. The method of claim 35, wherein the second ligand is an anti-CD154 mAb.

39. The method of claim 35, wherein the first ligand is a soluble CTLA4 molecule and the second ligand is an anti-CD154 mAb.

40. The method of claim 8, 15, 23 or 39, wherein soluble CTLA4 is CTLA4Ig, and the antibody that binds CD 154 is MR1.

41. The method of claim 8, 15, 23 or 39, wherein soluble CTLA4 is CTLA4Ig, and the antibody that binds CD154 is selected from a group consisting of ATCC HB11809, HB 11815, HB11816, HB 11817, HB 11819 HB 11821, and HB 11822.

42. A method of inhibiting rejection of a solid organ transplant in a subject having a transplanted tissue comprising
a) administering T cell depleted bone marrow cells;
b) administering busulfan to the subject; and
c) administering CTLA4Ig and a monoclonal antibody MR1 to the subject,
thereby inhibiting rejection of the solid organ or tissue/cellular transplant.

43. A method of inhibiting rejection of a solid organ transplant in a subject having a transplanted tissue comprising
a) administering T cell depleted bone marrow cells;
b) administering busulfan to the subject; and
c) administering CTLA4Ig and a monoclonal antibody consisting of ATCC HB111809, HB 11815, HB11816, HB 11817, HB 11819 HB 11821, and HB 11822, to the subject,
thereby inhibiting rejection of the solid organ or tissue/cellular transplant.


SEQUENCE LISTING (20 sequences)

SEQ ID NO: 1 - DNA, 1152 bp, Artificial Sequence
  Description of Artificial Sequence L104EIg sequence
atgggtgtactgctcacacagaggacgctgctcagtctggtccttgcactcctgtttccaagcatggcgagcatggcaatgcacgtggcccagcctgctgtggtactggccagcagccgaggcatcgctagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacgagggcataggcaacggaacccagatttatgtaattgatccagaaccgtgcccagattctgatcaggagcccaaatcttctgacaaaactcacacatccccaccgtccccagcacctgaactcctggggggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 383 aa, Artificial Sequence
  Description of Artificial Sequence L104EIg sequence
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 1152 bp, Artificial Sequence
  Description of Artificial Sequence L104EA29YIg sequence
atgggtgtactgctcacacagaggacgctgctcagtctggtccttgcactcctgtttccaagcatggcgagcatggcaatgcacgtggcccagcctgctgtggtactggccagcagccgaggcatcgctagctttgtgtgtgagtatgcatctccaggcaaatatactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacgagggcataggcaacggaacccagatttatgtaattgatccagaaccgtgcccagattctgatcaggagcccaaatcttctgacaaaactcacacatccccaccgtccccagcacctgaactcctggggggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 383 aa, Artificial Sequence
  Description of Artificial Sequence L104EA29YIg sequence
MGVLLTQRTLLSLVLALLFPSMASMAMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 1152 bp, Artificial Sequence
  Description of Artificial Sequence L104EA29LIg sequence
atgggtgtactgctcacacagaggacgctgctcagtctggtccttgcactcctgtttccaagcatggcgagcatggcaatgcacgtggcccagcctgctgtggtactggccagcagccgaggcatcgctagctttgtgtgtgagtatgcatctccaggcaaattgactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacgagggcataggcaacggaacccagatttatgtaattgatccagaaccgtgcccagattctgatcaggagcccaaatcttctgacaaaactcacacatccccaccgtccccagcacctgaactcctggggggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - protein, 383 aa, Artificial Sequence
  Description of Artificial Sequence L104EA29LIg sequence
MGVLLTQRTLLSLVLALLFPSMASMAMHVAQPAVVLASSRGIASFVCEYASPGKLTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 1152 bp, Artificial Sequence
  Description of Artificial Sequence L104EA29TIg sequence
atgggtgtactgctcacacagaggacgctgctcagtctggtccttgcactcctgtttccaagcatggcgagcatggcaatgcacgtggcccagcctgctgtggtactggccagcagccgaggcatcgctagctttgtgtgtgagtatgcatctccaggcaaaactactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacgagggcataggcaacggaacccagatttatgtaattgatccagaaccgtgcccagattctgatcaggagcccaaatcttctgacaaaactcacacatccccaccgtccccagcacctgaactcctggggggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 8 - protein, 383 aa, Artificial Sequence
  Description of Artificial Sequence L104EA29TIg sequence
MGVLLTQRTLLSLVLALLFPSMASMAMHVAQPAVVLASSRGIASFVCEYASPGKTTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1152 bp, Artificial Sequence
  Description of Artificial Sequence L104EA29WIg sequence
atgggtgtactgctcacacagaggacgctgctcagtctggtccttgcactcctgtttccaagcatggcgagcatggcaatgcacgtggcccagcctgctgtggtactggccagcagccgaggcatcgctagctttgtgtgtgagtatgcatctccaggcaaatggactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacgagggcataggcaacggaacccagatttatgtaattgatccagaaccgtgcccagattctgatcaggagcccaaatcttctgacaaaactcacacatccccaccgtccccagcacctgaactcctggggggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 10 - protein, 383 aa, Artificial Sequence
  Description of Artificial Sequence L104EA29WIg sequence
MGVLLTQRTLLSLVLALLFPSMASMAMHVAQPAVVLASSRGIASFVCEYASPGKWTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 636 bp, Homo sapiens
atgggtgtactgctcacacagaggacgctgctcagtctggtccttgcactcctgtttccaagcatggcgagcatggcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaat

SEQ ID NO: 12 - protein, 212 aa, Homo sapiens
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 13 - DNA, 1152 bp, Artificial Sequence
  Description of Artificial Sequence CTLA4Ig sequence
atgggtgtactgctcacacagaggacgctgctcagtctggtccttgcactcctgtttccaagcatggcgagcatggcaatgcacgtggcccagcctgctgtggtactggccagcagccgaggcatcgctagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgatcaggagcccaaatcttctgacaaaactcacacatccccaccgtccccagcacctgaactcctgggtggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 383 aa, Artificial Sequence
  Description of Artificial Sequence CTLA4Ig sequence
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 6 aa, Homo sapiens
MYPPPY

SEQ ID NO: 16 - DNA, 65 bp, Artificial Sequence
  Description of Artificial Sequence primer sequence encoding Oncostatin M-CTLA4 fusion
ctcagtctggtccttgcactcctgtttccaagcatggcgagcatggcaatgcacgtggcccagcc

SEQ ID NO: 17 - DNA, 33 bp, Artificial Sequence
  Description of Artificial Sequence primer sequence encoding CTLA4 sequence
tttgggctcctgatcagaatctgggcacggttg

SEQ ID NO: 18 - DNA, 72 bp, Artificial Sequence
  Description of Artificial Sequence primer sequence encoding Oncostatin M signal peptide sequence
ctagccactgaagcttcaccaatgggtgtactgctcacacagaggacgctgctcagtctggtccttgcactc

SEQ ID NO: 19 - DNA, 41 bp, Artificial Sequence
  Description of Artificial Sequence primer sequence from vector sequence
gaggtgataaagcttcaccaatgggtgtactgctcacacag

SEQ ID NO: 20 - DNA, 42 bp, Artificial Sequence
  Description of Artificial Sequence primer sequence encoding CTLA4 sequence
gtggtgtattggtctagatcaatcagaatctgggcacggttc